(19) United States
(12) Patent Application Publication
(10) Pub. No.: US 2009/0137793 A1
(43) Pub. Date: May 28, 2009

(54) DISACCHARIDE COMPOUNDS

(75) Inventor: Shang-cheng Hung, Taipei City (TW)

Correspondence Address: OCCHIUTI ROHLICEK & TSAO, LLP, 10 FAWCETT STREET, CAMBRIDGE, MA, 02138, US

(73) Assignee: Academia Sinica, Taipei (TW)

(21) Appl. No.: 12/273,768

(22) Filed: Nov. 19, 2008

Related U.S. Application Data
(60) Provisional application No. 60/988,924, filed on Nov. 19, 2007.

(51) Int. Cl. C07H 3/04; C07H 1/00

(52) U.S. Cl. 536/123.13; 536/124

(57) ABSTRACT

Disaccharide compounds used as building blocks for making heparin and heparan sulfate oligosaccharides. Also disclosed are methods for making these disaccharide compounds.

### CROSS REFERENCE TO RELATED APPLICATIONS

This application claims priority to U.S. Provisional Application Ser. No. 60/988,924 filed Nov. 19, 2007. The contents of the prior application are hereby incorporated by reference in their entireties.

### BACKGROUND

Heparin (HP) and heparan sulfate (HS), highly sulfonated and highly acidic, are structurally related linear polyanionic polysaccharides belonging to the family of glycosaminoglycans. HP occurs exclusively in mast cells. Exbiting high binding affinity with antithrombin III, it is widely used as an anticoagulant drug. HS is ubiquitously distributed on cell surfaces and in extracellular matrixes. It mediates various important physiological processes such as viral and bacterial infection, growth factor regulation, inflammatory response, angiogenesis, tumor metastasis, cell adhesion, and lipid metabolism.

Both HP and HS are biosynthesized through a unique pathway including formation of a polysaccharide backbone that contains alternate N-acetyl-R-D-glucosamine (GlcNAc) and b-D-glucuronic acid (GlcA) residues. This backbone is modified through a series of enzymatic processes to provide a large number of complex sequences resulting in microheterogeneity, for example, 48 di-, 48² tetra-, 48³ hexa-, 48⁴ octa-saccharides, etc. Among the 48 theoretically possible disaccharides, only 23 have been characterized so far. See Esko et al., Annu. ReV. Biochem. 2002, 71, 435-471.

Homogeneous HP and HS with well-defined configurations are necessary for identifying effective drug candidates. Such molecules are extremely difficult to acquire from natural sources. Over the past few years, several synthetic approaches have been documented to prepare specific HP and HS saccharide units. See Tabeur et al., Bioorg. Med. Chem. 1999, 7, 2003-2012; de Paz et al., ChemBioChem 2001, 2, 673-685; Prabhu et al.; Org. Lett. 2003, 5, 49754978; Orgueira et al., Chem.-Eur. J. 2003, 9, 140-169; Poletti et al., L.; Lay, L. Eur. J. Org. Chem. 2003, 2999-3024; Karst et al., Curr. Med. Chem. 2003, 10, 1993-2031; Lee et al., J. Am. Chem. Soc. 2004, 126, 476-477; Yu et al., Org. Lett. 2004, 6, 723-726; Lubineau et al., Chem.-Eur. J. 2004, 10, 4265-4282; Code et al., J. Am. Chem. Soc. 2005, 127, 3767-3773; and Noti et al., Chem. Biol. 2005, 12, 731-756.

### SUMMARY

In one aspect, this invention relates to disaccharide compounds that can be used as building blocks for making HP and HS oligosaccharides. These disaccharide compounds have either of the following general formulas:

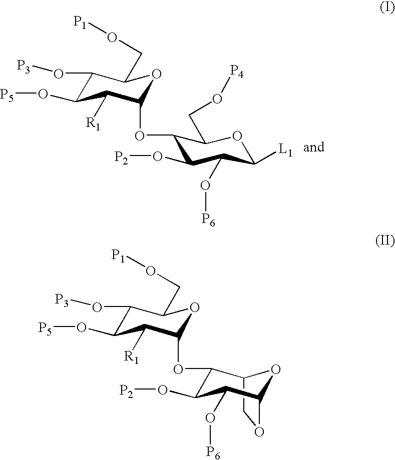

In these two formulas, each of P1-P6, independently, is a hydroxyl protecting group. More specifically, each of P₁, P₂, P₅, and P₆ independently, is arylalkyl (e.g., benzyl), arylcarbonyl (e.g., benzoyl), alkylcarbonyl, heteroarylakyl, or heteroarylcarbonyl; P₃ is aryl or heteroaryl (e.g., 2-naphthylmethyl); and P₄ is silyl (e.g., tert-butyldiphenylsilyl). L₁ is alkoxy, aryloxy, cycloalkyloxy, heteroaryloxy, heterocycloalkyloxy, alkylthio, arylthio (e.g., p-methylphenylthio), cycloalkylthio, heteroarylthio, heterocycloalkylthio, carbonyloxy, or halo; and R₁ is azido or amino optionally substituted by an amino protecting group (e.g., acetyl or barbobenzyloxy).

In another aspect, this invention relates to a method for synthesizing the above-described disaccharide compounds. This method includes reacting a monosaccharide compound having the general formula of

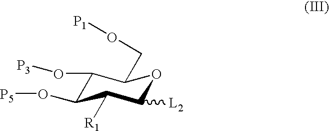

with one of the two compounds shown below:

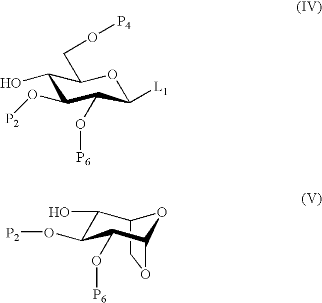

P₁ to P₆, L₁, and R₁ are defined above. L₂ is a leaving group (e.g., 2,2,2-trichloro-1-iminoethoxy). This reaction can be carried out in the presence of silver trifluoromethanesulfonate or trimethylsilyl trifluoromethanesulfonate.

The term “alkyl” refers to a straight or branched hydrocarbon, containing 1-6 carbon atoms. Examples of an alkyl group include, but are not limited to, methyl, ethyl, n-propyl, i-propyl, n-butyl, i-butyl, and 1-butyl. The term “alkoxy” refers to an O-alkyl radical. Examples of alkoxy groups include, but are not limited to, methoxy, ethoxyl, and butoxy. The term “aryl” refers to 6-carbon monocyclic, 10-carbon bicyclic, 14-carbon tricyclic aromatic ring systems. Examples of an aryl group include, but are not limited to, phenyl, naphthyl, and anthracenyl. The term “heteroaryl” refers to a moiety having at least one aromatic ring that contains at least one heteroatom. Examples of a heteroaryl moiety include furyl, furylene, fluorenyl, pyrrolyl, thienyl, oxazolyl, imidazolyl, thiazolyl, pyridyl, pyrimidinyl, quinazolinyl, and indolyl. The term “cycloalkyl” refers to a saturated non-aromatic cyclic hydrocarbon moiety. The term “silyl” refers to a silicon radical having three covalent bonds via which the silicon is also connected with three moieties selected from alkyl, aryl, cycloalyl, heterocyclocalyl, and heteroayl. Examples of a silyl group include, but are not limited to, trimethylsilyl, tert-butyldiphenylsilyl, tert-butyldimethylsilyl, and triisopropylsilyl.

A protecting group refer to a group that, upon attachment to an active moiety (e.g., hydroxyl or amino), prevents that moiety from interference with a subsequent reaction and can be removed by conventional methods after the reaction. Examples of a hydroxyl protecting group include, but are not limited to, alkyl, benzyl, allyl, trityl (i.e., triphenylmethyl), acyl (e.g., benzoyl, acetyl, or HOOC—X″—CO—, X″ being alkylene, alkenylene, cycloalkylene, or arylene), silyl (e.g., trimethylsilyl, triethylsilyl, and t-butyldimethylsilyl), alkoxylcarbonyl, aminocarbonyl (e.g., dimethylaminocarbonyl, methylethylaminocarbonyl, and phenylaminocarbonyl), alkoxymethyl, benzyloxymethyl, and alkylmercaptomethyl. Examples of an amino protecting group include, but are not limited to, alkyl, acyl, and silyl. Hydroxyl and amino protecting groups have been discussed in T. W. Greene and P. G. M. Wuts, Protective Groups in Organic Synthesis, 2nd. Ed., John Wiley and Sons (1991).

A leaving group can depart, upon direct displacement or ionization, with the pair of electrons from one of its covalent bonds (see, e.g., F. A. Carey and R. J. Sundberg, Advanced Organic Chemistry, 3^{rd} Ed. Plenum Press, 1990). Examples of a leaving group include, but are not limited to, 2,2,2-trichloro-1-iminoethoxy, trifluoroacetoxy (CF₃CO₂—), methanesulfonyloxy (CH₃SO₃—), p-toluenesulfonyloxy (ToISO₃—), iodide, bromide, and chloride.

The details of one or more embodiments of the invention are set forth in the description below. Other features, objects, and advantages of the invention will be apparent from the description and from the claims.

### DETAILED DESCRIPTION

The disaccharide compounds of this invention can be prepared from two common sugars, D-glucose and D-glucosamine, as outlined in Scheme 1 below. See also Lu et al., Organic Lett., 2006, 8(26), 5995-5998.

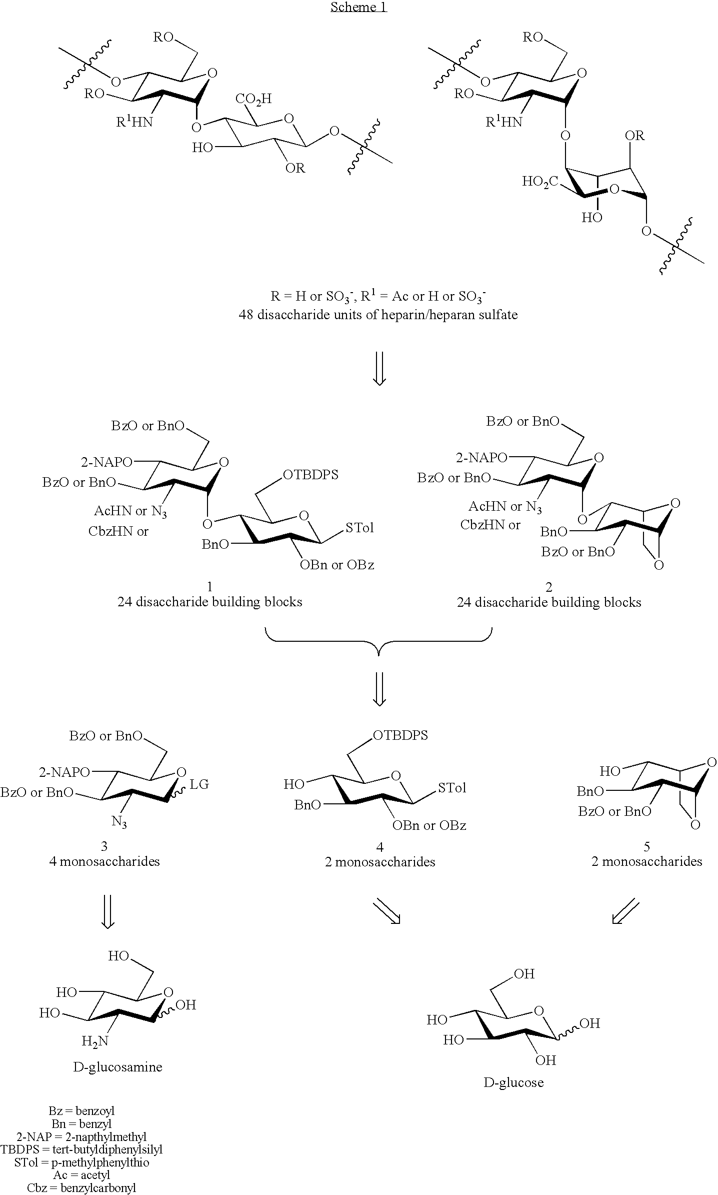

As shown above, hydroxyl groups of D-glucosamine and D-glucose are protected by various types of hydroxyl protecting groups to prevent a hydroxyl group from reacting with another substance during synthesis of the disaccharide compounds of this invention, and/or during synthesis of HP/HS oligosaccharides. Hydroxyl groups of the two monosaccharides that would ultimately be sulfonated can be protected by a suitable protecting group (e.g., Bz), and those that would remain free can be protected by a permanent protecting group (e.g., Bn). To exclusively generate 1,2 trans-glycosidic linkages during chain elongation, the same ester functionality at C2 is expected to offer anchimeric assistance (neighboring group participation), whereas benzyl ethers at such locations have the stereochemistry of glycosidation controlled via solvent effects (e.g., CH₃CN). See Schmidt et al., Synlett 1990, 694-696.

In addition, a temporary protecting group, such as TBDPS, is used to mask the primary hydroxyl at C6 so that at a later stage, the protecting group can be removed and the hydroxyl group can be transformed to a carboxylic acid. The hydroxyl attached to C4 is protected by a protecting group (e.g., 2-NAP) that allows mildly chemoselective deprotection for further elongation of the sugar chain. This protecting group can be sequentially or simultaneously removed along with Bn under hydrogenolytic conditions at a final termination process. See Spivey et al., Annu. Rep. Prog. Chem. Sect. B. 2002, 98: 41-60 and Xia et al., Tetrahedron Lett, 2000, 41, 169-172. The C2 amino group of D-glucosamine is protected as an azide due to its nonparticipating nature in coupling reactions. Azide, which predominantly leads a disaccharide building block to the α-anomeric conformation, can be readily transformed into acetamido (AcNH) and benzyloxycarbonylamino (NHCbz) groups. At a later stage, the —N₃ group can be selectively converted to the —NHSO₃⁻ unit via a combination of Staudinger reaction and N-sulfonation, whereas the —NHCbz is expected to reveal a free —NH₂ upon hydrogenolysis.

Also shown in Scheme 1, four D-glucosamine-derived glycosyl donors 3 can be each coupled with two D-glucopyranosyl 4-alcohols 4 and two L-idopyranosyl 4-alcohols 5 via Schmidt's trichloroacetimidate method (see Schmidt et al., Adv. Carbohydr. Chem. Biochem. 1994, 50, 21-123) to produce two sets of disaccharides, each containing eight disaccharides. These 16 disaccharides each can be converted to N-acetylated and N-Cbz-protected derivatives, generating a total of 48 disaccharide compounds (shown as 1 and 2). Note that 1,6-anhydro-β-L-idopyranosyl sugar 5, which can be prepared from D-glucose through C5 epimerization, serves as highly active glycosyl acceptors because of the rigid conformation and three equatorially substituted groups at C2, C3, and C4. It reacts with 4-hydroxy-free glucose 4 to form disaccharide 2. The 1,6-anhydro ring of 2 can be opened, and modification of functional groups and glycosylation of the corresponding L-idopyranosyl sugar at C6 and C1 can be conducted subsequently to produce the desired compounds.

Without further elaboration, it is believed that one skilled in the art can, based on the above description, utilize the present invention to its fullest extent. The following specific examples are, therefore, to be construed as merely illustrative, and not limitative of the remainder of the disclosure in any way whatsoever. All publications cited herein are incorporated by reference.

In the examples below, 48 exemplary disaccharide compounds were synthesized via the methods illustrated in Scheme 1.

### Preparation of Glycosyl Donors 15-18

Four glycosyl donors 15-18 were synthesized via the methods shown in Scheme 2 below:

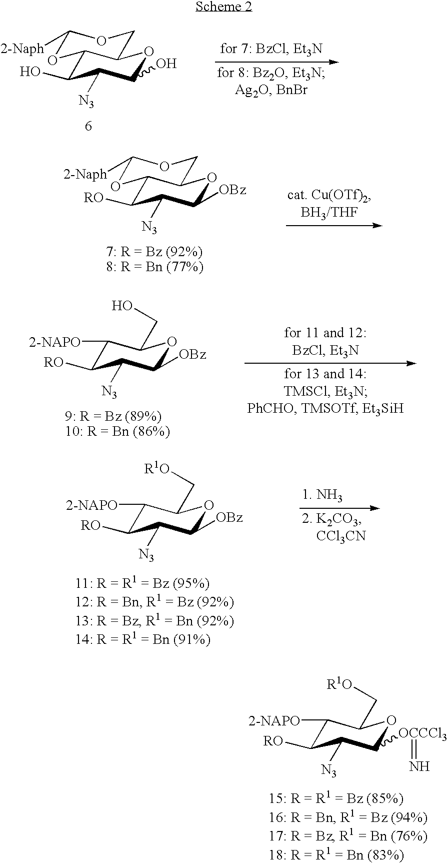

Synthesis of Compounds 7 and 8: A mixture of the 1,3-diol 6 (1.02 g, 2.97 mmol), triethylamine (2.5 mL, 17.82 mmol) and 4-(N,N-dimethylamino)pyridine (182 mg, 1.49 mmol) in dichloromethane (10 mL) was cooled to 0° C. under nitrogen. Benzoyl chloride (1.1 mL, 9.5 mmol) was slowly added to the solution, the ice-bath was removed, and the mixture was kept stirring at room temperature. After the starting material was completely consumed, methanol (2 mL) was added to quench the reaction, and the resulting solution was evaporated under reduced pressure. Water was added to the solid residue, the mixture was extracted with ethyl acetate, and the combined organic layers were sequentially washed with saturated NaHCO₃ (aq) and brine. The organic layer was dried over anhydrous MgSO₄, filtered, and concentrated in vacuo. The residue was purified by flash column chromatography (EtOAc/Hex=1/5) on silica gel to get the benzoylated compound 7 (1.50 g, 92%). ¹H NMR (500 MHz, CDCl₃) δ 8.12-8.08 (m, 4H, BzH), 7.87 (s, 1H, ArH), 7.80-7.76 (m, 3H, ArH), 7.62 (tt, J=7.5, 1.1 Hz, 1H, ArH), 7.58 (tt, J=7.5, 1.1 Hz, 1H, ArH), 7.50-7.42 (m, 7H, ArH), 6.00 (d, J=8.4 Hz, 1H, H-1), 5.67 (s, 1H, NaphCH), 5.62 (t, J=9.7 Hz, 1H, H-3), 4.47 (dd, J=15.5, 9.9 Hz, 1H, H-6a), 4.02 (dd, J=9.7, 8.4 Hz, 1H, H-2), 3.95 (t, J=9.7 Hz, 1H, H-4), 3.85 (m, 2H, H-5, H-6b); ¹³C NMR (125 MHz, CDCl₃) δ 165.2 (C), 164.2 (C), 134.1 (CH), 133.9 (C), 133.6 (C), 133.4 (CH), 132.8 (C), 130.1 (CH), 129.9 (CH), 129.3 (C), 128.7 (CH), 128.5 (CH), 128.39 (C), 128.35 (CH), 128.1 (CH), 127.6 (CH), 126.4 (CH), 126.1 (CH), 125.7 (CH), 123.6 (CH), 101.9 (CH), 93.9 (CH), 78.7 (CH), 71.9 (CH), 68.4 (CH₂), 67.3 (CH), 64.2 (CH); HRMS (FAB, MH⁺) calcd for C₃₁H₂₆N₃O₇ 552.1771, found 552.1764.

The 1,3-diol 6 (30.0 g, 87.4 mmol) and benzoic anhydride (21.7 g, 96.1 mmol) were dissolved in dichloromethane (300 mL), and the reaction flask was immersed in an ice bath. Triethylamine (110 mL, 787 mmol) was added to the solution, the ice bath was removed, and the mixture was kept stirring overnight. Methanol (25 mL) was added to quench the reaction, the mixture was stirred for another 30 min, and the resulting solution was evaporated under reduced pressure. Water was added to the solid residue, the mixture was extracted with ethyl acetate, and the combined organic layers were sequentially washed with saturated NaHCO₃ (aq) and brine. The organic portion was dried over anhydrous MgSO₄, filtered, and concentrated in vacuo to give a residue, which was purified by flash column chromatography (EtOAc/Hex=1/3) on silica gel to furnish the O1-benzoylated 3-alcohol (36.4 g, 93%).

To a mixture of this 3-alcohol (14.0 g, 0.03 mol) and silver(I) oxide (14.5 g, 0.06 mol) in anhydrous dichloromethane (140 mL) was added benzyl bromide (5.6 mL, 0.05 mol) at 0° C. under nitrogen. The reaction temperature was gradually warmed up to room temperature, and the solution was kept stirring for another 2 days. The resulting mixture was filtered through celite, the filtrate was concentrated in vacuo, and the crude residue was purified by flash column chromatography (EtOAc/Hex=1/6) on silica gel to provide the product 8 (14.0 g, 83%) as a white solid. ¹H NMR (400 MHz, CDCl₃) δ 8.11 (dd, J=8.4, 1.3 Hz, 2H, BzH), 7.98 (s, 1H, ArH), 7.90-7.85 (m, 3H, ArH), 7.64-7.58 (m, 2H, ArH), 7.53-7.47 (m, 4H, ArH), 7.42-7.39 (m, 2H, ArH), 7.36-7.28 (m, 3H, ArH), 5.81 (d, J=8.4 Hz, 1H, H-1), 5.76 (s, 1H, NaphCH), 4.99 (d, J=11.2 Hz, 1H, CH₂Ph), 4.86 (d, J=11.2 Hz, 1H, CH₂Ph), 4.45 (dd, J=10.5, 5.0 Hz, 1H, H-6a), 3.88-3.77 (m, 4H, H-2, H-3, H-4, H-6b), 3.71-3.61 (m, 1H, H-5); ¹³C NMR (75 MHz, CDCl₃) δ 164.45 (C), 137.57 (C), 134.30 (C), 133.93 (CH), 133.64 (C), 132.86 (C), 130.09 (CH), 128.58 (CH), 128.41 (CH), 128.38 (C), 128.18 (CH), 128.15 (C), 128.00 (CH), 127.71 (CH), 126.52 (CH), 126.26 (CH), 125.52 (CH), 123.56 (CH), 101.59 (CH), 93.62 (CH), 81.46 (CH), 79.17 (CH), 75.05 (CH₂), 68.39 (CH₂), 67.00 (CH), 65.19 (CH); HRMS (FAB, MH⁺) calcd for C₃₁H₂₈N₃O₆ 538.1978, found 538.1981.

Synthesis of Compounds 9 and 10: A 1 M solution of borane/tetrahydrofuran complex in tetrahydrofuran (9 mmol) was added to the acetal 7 (1.8 mmol) at room temperature under nitrogen. The mixture was stirred for 10 min, and freshly dried copper(II) trifluoromethanesulfonate (0.09 mmol) was added to the solution. After the starting material was completely consumed, the mixture was cooled down to 0° C., and the reaction was quenched by sequential additions of triethylamine (1.8 mmol) and methanol (3.2 mL, caution: hydrogen gas was evolved). The resulting mixture was concentrated at reduced pressure followed by coevaporation with methanol. The residue was purified by flash column chromatographyon silica gel to give the 6-alcohols (9: 89%, 10: 86%). 9: ¹H NMR (500 MHz, CDCl₃) δ 8.07 (dd, J=8.3, 1.1 Hz, 2H, BzH), 7.95 (dd, J=8.3, 1.1 Hz, 2H, BzH), 7.67-7.64 (m, 2H, BzH), 7.61-7.51 (m, 4H, BzH), 7.47-7.44 (m, 2H, ArH), 7.41-7.35 (m, 4H, ArH), 7.25-7.22 (m, 1H, ArH), 5.89 (d, J=8.4 Hz, 1H, H-1), 5.50 (dd, J=10.0, 9.4 Hz, 1H, H-3), 4.78, 4.72 (ABq, J=11.3 Hz, 2H, CH₂Naph), 3.99 (t, J=9.4 Hz, 1H, H-4), 3.98 (ddd, J=12.5, 4.9, 2.8 Hz, 1H, H-6a), 3.85 (dd, J=10.0, 8.4 Hz, 1H, H-2), 3.84 (ddd, J=12.5, 8.4, 2.8 Hz, 1H, H-6b), 3.72 (dt, J=9.4, 2.8 Hz, 1H, H-5), 1.83 (dd, J=8.4, 4.9 Hz, 1H, OH-6); ¹³C NMR (125 MHz, CDCl₃) δ 165.3 (C), 164.4 (C), 134.5 (C), 134.0 (CH), 133.5 (CH), 133.04 (C), 132.98 (C), 130.1 (CH), 129.8 (CH), 129.2 (C), 128.6 (CH), 128.5 (C), 128.4 (CH), 128.3 (CH), 127.9 (CH), 127.7 (CH), 127.2 (CH), 126.1 (CH), 126.00 (CH), 125.97 (CH), 93.6 (CH), 76.3 (CH), 74.9 (CH₂), 74.7 (CH), 74.5 (CH), 63.8 (CH), 61.0 (CH₂); HRMS (FAB, M⁺) calcd for C₃₁H₂₇N₃O₇ 553.1849, found 553.1842. 10: ¹H NMR (400 MHz, CDCl₃) [symbol] 8.10 (d, J=8.5 Hz, 2H, BzH), 7.84-7.78 (m, 3H, ArH), 7.73 (s, 1H, ArH), 7.61 (dt, J=7.4, 1.2 Hz, 1H, ArH), 7.50-7.45 (m, 4H, ArH), 7.42-7.31 (m, 6H, ArH), 5.75 (d, J=8.2 Hz, 1H, H-1), 5.02 (d, J=11.2 Hz, 1H, CH₂Ar), 4.96-4.85 (m, 31H, CH₂Ar), 3.92 (dd, J=12.3, 2.4 Hz, 1H, H-6a), 3.79-3.63 (m, 4H, H-2, H-3, H-4, H-6b), 3.59 (ddd, J=9.7, 3.8, 2.4 Hz, 1H, H-5), 1.77 (s, 1H, OH-6); ¹³C NMR (75 MHz, CDCl₃) δ 164.60 (C), 137.64 (C), 135.05 (C), 133.87 (CH), 133.20 (C), 133.00 (C), 130.06 (CH), 128.65 (C), 128.56 (CH), 128.47 (C), 128.30 (CH), 128.03 (CH), 127.95 (CH), 127.91 (CH), 127.68 (CH), 126.73 (CH), 126.20 (CH), 126.06 (CH), 125.72 (CH), 93.52 (CH), 82.92 (CH), 76.88 (CH), 76.31 (CH), 75.69 (CH₂), 75.14 (CH₂), 65.51 (CH), 61.22 (CH₂); HRMS (FAB, MH⁺) calcd for C₃₁H₃₀N₃O₆ 540.2134, found 540.2148.

Synthesis of Compounds 11 and 12: Following the procedure for the preparation of compound 7 gave the products 11 (95%) and 12 (92%), respectively. 11: ¹H NMR (400 MHz, CDCl₃) δ 8.07 (dd, J=8.3, 1.2 Hz, 2H, BzH), 8.04 (dd, J=8.2, 1.2 Hz, 2H, BzH), 8.00 (dd, J=8.2, 1.2 Hz, 2H, BzH), 7.63-7.34 (m, 115H, ArH), 7.21 (dd, J=8.4, 1.5 Hz, 1H, ArH), 5.93 (d, J=8.4 Hz, 1H, H-1), 5.58 (dd, J=10.1, 8.4 Hz, 1H, H-3), 4.72, 4.67 (ABq, J=11.2 Hz, 2H, CH₂Naph), 4.65 (dd, J=12.2, 2.0 Hz, 1H, H-6a), 4.60 (dd, J=12.2, 3.5 Hz, 1H, H-6b), 4.04-3.98 (m, 2H, H-4, H-5), 3.94 (dd, J=10.1, 8.4 Hz, 1H, H-2); ¹³C NMR (100 MHz, CDCl₃) δ 166.0 (C), 165.2 (C), 164.2 (C), 134.0 (C), 133.9 (CH), 133.5 (CH), 133.1 (CH), 133.0 (C), 132.9 (C), 130.1 (CH), 129.8 (CH), 129.7 (CH), 129.6 (C), 129.2 (C), 128.6 (C), 128.53 (CH), 128.50 (CH), 128.42 (CH), 128.36 (CH), 127.8 (CH), 127.6 (CH), 127.3 (CH), 93.6 (CH), 75.1 (CH), 74.80 (CH), 74.76 (CH₂), 74.2 (CH), 63.8 (CH), 62.8 (CH₂); HRMS (FAB, M⁺) calcd for C₃₈H₃₁N₃O₈ 657.2111, found 657.2106. 12:1 H NMR (400 MHz, CDCl₃) δ 8.10 (dd, J=8.5, 1.3 Hz, 2H, BzH), 7.93 (dd, J=8.4, 1.3 Hz, 2H, BzH), 7.77-7.70 (m, 31H, ArH), 7.66 (s, 1H, ArH), 7.59 (t, J=7.4 Hz, 1H, ArH), 7.53-7.33 (m, 13H, ArH), 5.79 (d, J=8.4 Hz, 1H, H-1), 5.03 (d, J=11.0 Hz, 1H, CH₂Naph), 5.00 (d, J=10.6 Hz, 1H, CH₂Ph), 4.91 (d, J=10.6 Hz, 1H, CH₂Ph), 4.82 (d, J=11.0 Hz, 1H, CH₂Naph), 4.62 (dd, J=12.2, 1.6 Hz, 1H, H-6a), 4.53 (dd, J=12.2, 3.4 Hz, 1H, H-6b), 3.86-3.79 (m, 3H, H-2, H-4, H-5), 3.71 (dd, J=9.5, 8.2 Hz, 1H, H-3); ¹³C NMR (100 MHz, CDCl₃) δ 165.96 (C), 164.50 (C), 137.47 (C), 134.62 (C), 133.80 (CH), 133.11 (C), 133.02 (CH), 130.10 (CH), 129.60 (CH), 128.71 (C), 128.58 (CH), 128.50 (CH), 128.41 (CH), 128.28 (CH), 128.19 (CH), 128.12 (CH), 127.86 (CH), 127.64 (CH), 126.97 (CH), 126.14 (CH), 126.05 (CH), 125.74 (CH), 93.47 (CH), 83.30 (CH), 76.88 (CH), 75.90 (CH₂), 75.18 (CH₂), 74.12 (CH), 65.57 (CH), 62.81 (CH₂); HRMS (FAB, M⁺) calcd for C₃₈H₃₃N₃O₇ 643.2319, found 643.2333.

Synthesis of Compounds 13 and 14: To a solution of the 6-alcohol (9 or 10, 0.18 mmol) and triethylamine (0.72 mmol) in dichloromethane (1 mL) was slowly added chlorotrimethyl-silane (0.36 mmol) at 0° C. under nitrogen. The ice-bath was removed, the reaction was kept stirring at room temperature for 8 h, and the mixture was evaporated under reduced pressure. The residue was diluted with hexane (10 mL), the mixture was filtered through paper, and the solid was washed by hexane. The filtrate was concentrated in vacuo, and the crude trimethylsilyl ether was used in the next reaction without further purification.

A mixture of this crude trimethylsilyl ether (0.17 mmol), freshly dried 3 Å molecular sieves (108 mg), benzaldehyde (0.72 mmol), triethylsilane (0.26 mmol), and dichloromethane (1 mL) was stirred at room temperature for 30 min under nitrogen. The mixture was cooled down to −78° C., and trimethylsilyl trifluoromethanesulfonate (0.03 mmol) was slowly added to the solution. After stirring for 3 h, the reaction was quenched with saturated NaHCO₃ (aq), the whole mixture was filtered through celite, and the filtrate was extracted with ethyl acetate (3×6 mL). The combined organic layers were washed with brine, dried over magnesium sulfate, filtered, and concentrated in vacuo. The residue was purified by flash column chromatography (EtOAc/Hex=1/5) on silica gel to afford the desired ether (13: 92% and 14: 91%). 13: ¹H NMR (400 MHz, CDCl₃) δ 8.09 (dd, J=8.4, 1.1 Hz, 2H, BzH), 7.93 (dd, J=8.4, 1.1 Hz, 2H, BzH), 7.66-7.26 (m, 17H, ArH), 7.10 (dd, J=8.4, 1.2 Hz, 1H, ArH), 5.86 (d, J=8.5 Hz, 1H, H-1), 5.48 (t, J=9.8 Hz, 1H, H-3), 4.67, 4.50 (ABq, J=12.0, 2H, CH₂Ar), 4.64, 4.61 (ABq, J=12.0, 2H, CH₂Ar), 4.09 (t, J=9.8 Hz, 1H, H-4), 3.89 (dd, J=9.8, 8.5 Hz, 1H, H-2), 3.87-3.77 (m, 3H, H-5, H-6a, H-6b); ¹³C NMR (100 MHZ, CDCl₃) δ 165.3 (C), 164.4 (C), 137.6 (C), 134.6 (C), 133.9 (CH), 133.3 (CH), 133.0 (C), 132.9 (C), 130.1 (CH), 129.7 (CH), 129.2 (C), 128.7 (C), 128.6 (CH), 128.5 (CH), 128.4 (CH), 128.2 (CH), 127.9 (CH), 127.8 (CH), 127.6 (CH), 127.0 (CH), 126.0 (CH), 125.9 (CH), 93.7 (CH), 75.8 (CH), 75.0 (CH), 74.63 (CH), 74.60 (CH₂), 73.7 (CH₂), 67.7 (CH₂), 63.8 (CH); HRMS (FAB, M⁺) calcd for C₃₈H₃₃N₃O₇ 643.2319, found 643.2307. 14: ¹H NMR (500 MHz, CDCl₃) δ 8.15 (dd, J=8.1 Hz, 2H, ArH), 7.86-7.82 (m, 1H, ArH), 7.81-7.76 (m, 2H, ArH), 7.64-7.59 (m, 2H, ArH), 7.56-7.45 (m, 5H, ArH), 7.42-7.25 (m, 10H, ArH), 5.77 (d, J=8.5 Hz, 1H, H-1), 4.99 (d, J=11.1 Hz, 1H, CH₂Ar), 4.96, 4.91 (ABq, J=11.1, 2H, CH₂Ar), 4.76 (d, J=11.1 Hz, 1H, CH₂Ar), 4.64, 4.48 (ABq, J=12.1, 2H, CH₂Ar), 3.93 (t, J=9.3 Hz, 1H, H-3), 3.86-3.75 (m, 3H, H-4, H-6a, H-6b), 3.72-3.67 (m, 1H, H-5), 3.65 (dd, J=9.3, 8.5 Hz, 1H, H-2); ¹³C NMR (125 MHZ, CDCl₃), δ 164.6 (C), 137.72 (C), 137.69 (C), 135.2 (C), 133.7 (CH), 133.2 (C), 133.0 (C), 130.5 (CH), 130.1 (CH), 128.8 (CH), 128.5 (CH), 128.4 (CH), 128.3 (CH), 128.1 (CH), 128.0 (CH), 127.91 (CH), 127.86 (CH), 127.7 (CH), 127.6 (CH), 126.5 (CH), 126.1 (CH), 125.9 (CH), 125.7 (CH), 93.6 (CH), 83.1 (CH), 77.1 (CH), 75.8 (CH), 75.6 (CH₂), 75.0 (CH₂), 73.5 (CH₂), 67.9 (CH₂), 65.5 (CH).

Synthesis of Compounds 15-18: Ammonia gas was passed through a solution of the 1-benzoate 11, 12, 13 or 14 (1 equiv) in a mixed solvent THF and MeOH (7/3 ratio, 50 mL/g) for 0.5 h at 0° C., the gas valve was closed, and the mixture was kept stirring at the same temperature for another 8 h. The solvent was removed under reduced pressure, and the residue was purified by flash column chromatography on silica gel to give the corresponding 1-alcohol.

To a mixture of this 1-alcohol (1 equiv) in dichloromethane (10 mL/g) was consecutively added potassium carbonate (5 equiv) and trichloroacetonitrile (10 equiv) at room temperature under nitrogen. After stirring for 16 h, the reaction solution was filtered through celite, the filtrate was diluted by water, and the mixture was extracted with ethyl acetate (three times). The combined organic layers were washed with brine, dried over magnesium sulfate, filtered, and concentrated in vacuo to furnish the crude trichloroacetimidate (15: 85%; 16: 94%; 17: 76%; and 18: 83%), which was directly used for the ensuing reaction without further purification.

### Preparation of Thioglycosides 21 and 24

Thioglycosides 21 and 24 were prepared by the methods shown in Scheme 3 below:

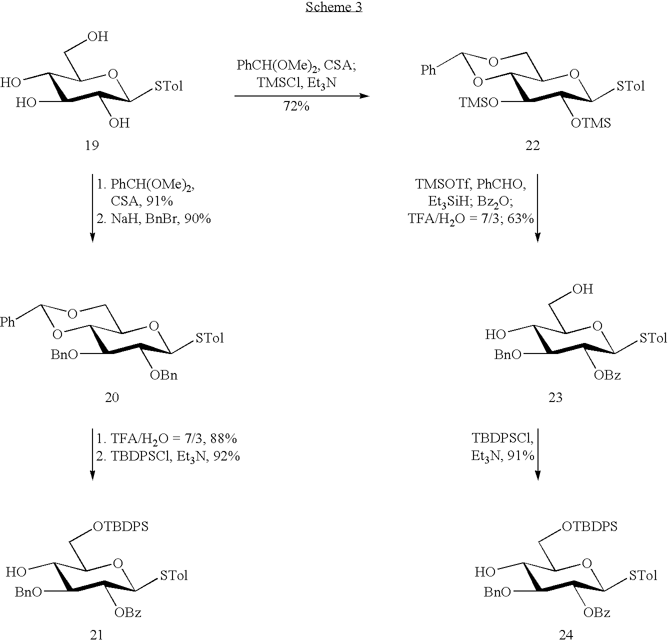

Synthesis of Compound 21: Tetraol 19 underwent sequential 4,6-O-benzylidenation (91%) and 2,3-di-O-benzylation (90%) to yield the ether derivative 20. A mixture of compound 20 (1.86 g, 3.35 mmol) and 70% TFA (aq) (20 mL) was stirred at room temperature for 0.5 h. The solvent was removed under reduced pressure, and the residue was purified by flash column chromatography (EtOAc/Hex=2/3) to give the 4,6-diol (1.38 g, 88%).

To a mixture of this diol (1.00 g, 2.14 mmol), triethylamine (0.66 mL, 4.70 mmol), cat. 4-(N,N-dimethylamino)pyridine (26 mg, 0.21 mmol) in dichloromethane (10 mL) was slowly added TBDPSCl (0.6 mL, 2.35 mmol) at room temperature under nitrogen. After stirring for 8 h, the reaction solution was diluted with ethyl acetate (3×15 mL), and the mixture was consecutively washed by water and brine. The organic layer was dried over MgSO₄, filtered, and concentrated in vacuo to get a residue, which was purified by flash column chromatography (EtOAc/Hex=1/8) on silica gel to provide the expected 4-alcohol 21 (0.69 g, 92%). ¹H NMR (500 MHz, CDCl₃) δ 7.72-7.68 (m, 4H, ArH), 7.47-7.26 (m, 18H, ArH), 7.00 (d, J=8.0 Hz, 2H, ArH), 4.89, 4.71 (ABq, J=10.3 Hz, 2H, CH₂Ph), 4.88, 4.78 (ABq, J=11.4 Hz, 2H, CH₂Ph), 4.60 (d, J=9.6 Hz, 1H, H-1), 3.94 (dd, J=11.0, 3.9 Hz, 1H, H-6a), 3.90 (dd, J=11.0, 4.6 Hz, 1H, H-6b), 3.71 (t, J=9.0 Hz, 1H, H-4), 3.52 (t, J=9.0 Hz, 1H, H-3), 3.44 (dd, J=9.6, 9.0 Hz, 1H, H-2), 3.38-3.33 (m, 1H, H-5), 2.48-2.40 (brs, 1H, OH-4), 2.28 (s, 3H, CH₃), 1.07 (s, 9H, t-Bu); ¹³C NMR (100 MHz, CDCl₃) δ 138.4 (C), 138.1 (C), 137.4 (C), 135.6 (CH), 135.5 (CH), 133.0 (C), 132.9 (C), 132.3 (CH), 129.9 (C), 129.67 (CH), 129.65 (CH), 129.6 (CH), 128.5 (CH), 128.3 (CH), 128.1 (CH), 127.9 (CH), 127.8 (CH), 127.72 (CH), 127.66 (CH), 87.7 (CH), 86.3 (CH), 80.1 (CH), 79.1 (CH), 75.4 (CH₂), 75.1 (CH₂), 70.9 (CH), 64.0 (CH₂), 26.8 (t-Bu), 21.0 (CH₃), 19.1 (C); HRMS (FAB, MNa⁺) calcd for C₄₃H₄₈O₅SiSNa 727.2889, found 727.2881.

Synthesis of Compound 23: A mixture of the trimethylsilyl ether 22 (150 mg, 0.30 mmol), freshly dried 3 Å molecular sieves (225 mg), benzaldehyde (33 μL, 0.33 mmol), triethylsilane (51 μL, 0.33 mmol), and dichloromethane (3 mL) was stirred at room temperature for 30 min under nitrogen. The reaction flask was cooled down to −78° C., trimethylsilyl trifluoromethanesulfonate (9 μL, 0.03 mmol) was slowly added to the solution, the mixture was kept stirring for another 4 h, the temperature was gradually warmed up to 0° C., and benzoic anhydride (195 mg, 0.87 mmol), and trimethylsilyl trifluoromethanesulfonate (42 μL, 0.24 mmol) were sequentially added to the reaction mixture. After stirring for 8 h, 70% TFA (aq) (3 mL) was added to the resulting solution, ice-bath was removed, and the reaction was continuously stirred at room temperature for another 4 h. The reaction was quenched by saturated NaHCO₃ (aq), and the aqueous phase was extracted with ethyl acetate (3×6 mL). The combined organic layers were washed with brine, dried over magnesium sulfate, filtered, and concentrated in vacuo. The residue was purified by flash column chromatography (EtOAc/Hex=1/1) on silica gel to afford the desired 4,6-diol 23 (90 mg, 63%) as a white solid. ¹H NMR (400 MHz, CDCl₃) δ 8.07-8.05 (m, 2H, ArH), 7.61-7.57 (m, 1H, ArH), 7.49-7.44 (m, 2H, ArH), 7.30 (d, J=8.1 Hz, 2H, ArH), 7.21-7.15 (m, 5H, ArH), 7.06 (d, J=7.9 Hz, 2H, ArH), 5.20 (dd, J=10.0, 9.0 Hz, 1H, H-2), 4.75 (d, J=10.0 Hz, 1H, H-1), 4.70 (d, J=11.4 Hz, 1H, CH₂Ph), 4.55 (d, J=11.4 Hz, 1H, CH₂Ph), 3.90 (ddd, J=12.0, 6.6, 3.4 Hz, 1H, H-6a), 3.81-3.75 (m, 1H, H-6b), 3.71-3.66 (m, 2H, H-3, H-4), 3.46-3.42 (m, 1H, H-5), 2.37 (d, J=2.2 Hz, 1H, OH-4), 2.30 (s, 3H, CH₃), 2.11 (t, J=6.6 Hz, 1H, OH-6); ¹³C NMR (100 MHz, CDCl₃) [symbol] 165.19 (C), 138.37 (C), 137.63 (C), 133.33 (CH), 133.12 (CH), 129.85 (CH), 129.69 (CH), 128.60 (C), 128.51 (CH), 128.49 (CH), 128.01 (CH), 86.60 (CH), 83.86 (CH), 79.36 (CH), 74.76 (CH₂), 72.36 (CH), 70.26 (CH), 62.56 (CH₂), 21.11 (CH₃); HRMS (FAB, MH⁺) calcd for C₂₇H₂₈O₆S 481.1685, found 481.1696.

Compound 23 was protected at the O6 position to afford the corresponding silyl ether 24 in 91% yield.

Preparation of 1,6-anhydro-b-L-idopyranosyl-4-alcohols 27 and 30

1,6-anhydro-β-L-idopyranosyl 4-alcohols 27 and 30 were prepared by the method shown in Scheme 4.

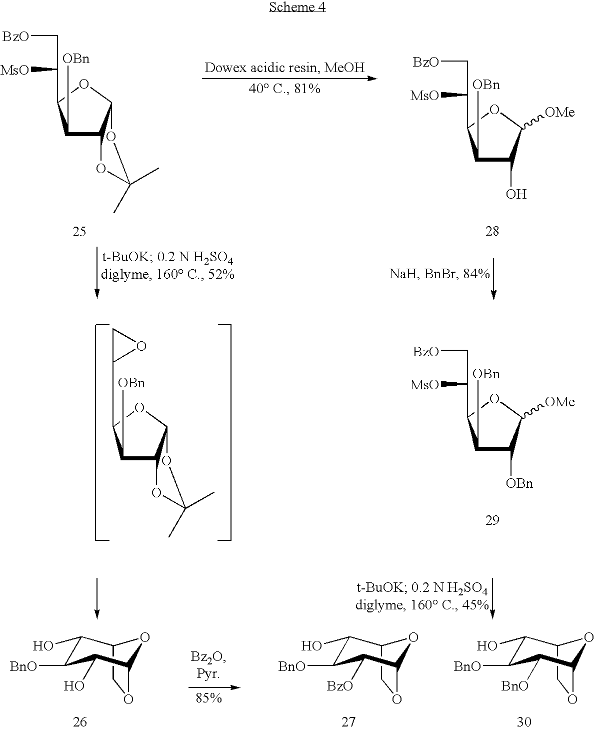

Synthesis of Compound 26: Compound 25 was prepared from diacetone R-D-glucose as described in Lee et al., 2004. To a solution of 25 (1.20 g, 2.44 mmol) in a mixed solvent of CH₂Cl₂ and t-BuOH (2/1 ratio, 18 mL) was added potassium t-butoxide (0.60 g, 5.36 mmol) at 0° C. under nitrogen. After stirring for 16 h, the reaction was acidified with 0.6 N H₂SO₄ (aq) (ca. 4.5 mL) and the flask was equipped with a simple distillation head to evaporate CH₂Cl₂ and t-BuOH under reduced pressure. 0.6 N H₂SO₄ (aq) (5 mL) and diglyme (10 mL) were added to the resulting solution, and the mixture was kept stirring at 160° C. for another 16 h. After cooling to room temperature, the reaction was neutralized with 3 N NaOH (aq) (2 mL), and the solvent was removed on rotary evaporator under vacuum. Water (10 mL) was added to the residue, and the mixture was extracted with EtOAc (3×10 mL). The combined organic layers were washed with brine, dried over MgSO₄, filtered, and concentrated in vacuo. The residue was purified by flash column chromatography (EtOAc/Hex=1/1) to give a white solid, which was further recrystallized via vapor diffusion method to provide 26 (0.32 g, 52%) as colorless crystals. ¹H NMR (400 MHz, CDCl₃) δ 7.36-7.28 (m, 5H, ArH), 5.27 (d, J=1.8 Hz, 1H, H-1), 4.93 (d, J=11.7 Hz, 1H, CH₂Ph), 4.72 (d, J=11.7 Hz, 1H, CH₂Ph), 4.41 (t, J=4.9 Hz, 1H, H-5), 4.01 (d, J=7.8 Hz, 1H, H-6a), 3.85 (ddd, J=7.8, 4.9, 3.3 Hz, 1H, H-4), 3.71 (dd, J=7.8, 4.9 Hz, 1H, H-6b), 3.64 (td, J=7.8, 1.8 Hz, 1H, H-2), 3.37 (t, J=7.8 Hz, 1H, H-3), 2.09 (d, J=3.3 Hz, 1H, OH-4), 1.89 (d, J=7.8 Hz, 1H, OH-2); ¹³C NMR (75 MHz, CDCl₃) δ 138.4 (C), 128.7 (CH), 128.0 (CH), 127.9 (CH), 101.8 (CH), 84.2 (CH), 75.4 (CH), 75.0 (CH), 74.5 (CH₂), 71.1 (CH), 65.1 (CH₂); HRMS (FAB, M⁺) calcd for C₁₃H₁₆O₅ 252.0997, found 252.0992.

Synthesis of Compound 27: To a solution of 26 (3.00 g, 11.9 mmol) in dichloromethane (35 mL) was consecutively added pyridine (3.0 mL, 35.7 mmol) and benzoyl chloride (1.4 mL, 12.5 mmol) at 0° C. under nitrogen. After stirring for 4 h, the reaction was quenched with MeOH (2 mL), and the solvent was evaporated at reduced pressure. The mixture was dissolved in EtOAc (30 mL), and the solution was sequentially washed with 2 N HCl (aq), saturated NaHCO₃ (aq), water, and brine. The organic layer was dried over anhydrous MgSO₄, filtered, and concentrated in vacuo. The residue was recrystallized from EA/Hex=1/4 to afford the 4-alcohol 27 (3.60 g, 85%) as a white solid. ¹H NMR (400 MHz, CDCl₃) δ 8.08 (dd, J=7.6, 1.3 Hz, 2H, BzH), 7.59 (tt, J=7.6, 1.3 Hz, 1H, BzH), 7.46 (t, J=7.6 Hz, 2H, BzH), 7.29-7.24 (m, 5H, ArH), 5.53 (d, J=1.6 Hz, 1H, H-1), 5.07 (dd, J=8.2, 1.6 Hz, 1H, H-2), 4.80 (d, J=11.6 Hz, 1H, CH₂Ph), 4.65 (d, J=11.6 Hz, 1H, CH₂Ph), 4.51 (t, J=4.6 Hz, 1H, H-5), 4.15 (d, J=7.5 Hz, 1H, H-6a), 4.00 (ddd, J=8.2, 4.6, 3.0 Hz, 1H, H-4), 3.87 (t, J=8.2 Hz, 1H, H-3), 3.76 (dd, J=7.5, 4.6 Hz, 1H, H-6b), 2.22 (d, J=3.0 Hz, 1H, OH-4); ¹³C NMR (100 MHz, CDCl₃) δ 165.77 (C), 137.94 (C), 133.38 (CH), 129.81 (CH), 129.40 (C), 128.53 (CH), 128.45 (CH), 127.96 (CH), 127.84 (CH), 99.41 (CH), 80.24 (CH), 76.71 (CH), 75.07 (CH), 74.58 (CH₂), 71.33 (CH), 65.27 (CH); HRMS (FAB, MH⁺) calcd for C₂₀H₂₁O₆ 357.1338, found 357.1353.

Synthesis of Compound 28: A mixture of 25 (1.00 g, 2.10 mol) and Dowex 500wx8-200 resin (2.2 g) in MeOH (10 mL) was stirred at 40° C. for 16 h under nitrogen. After cooling down to room temperature, the reaction mixture was filtered, the filtrate was concentrated in vacuo, and the residue was purified by flash column chromatography (EtOAc/Hex=1/1) on silica gel to get the product 28 (739 mg, 81%). ¹H NMR (400 MHz, CDCl₃) δ 8.08 (dd, J=7.1, 1.4 Hz, 2H, BzH), 7.63-7.55 (m, 1H, BzH), 7.51-7.43 (m, 2H, BzH), 7.42-7.25 (m, 5H, ArH), 5.37 (td, J=6.1, 2.2 Hz, 1H, H-5), 5.05 (d, J=4.4 Hz, 1H, H-1), 4.79 (dd, J=12.6, 2.2 Hz, 1H, H-6a), 4.75 (d, J=11.5 Hz, 1H, CH₂Ph), 4.68 (d, J=11.5 Hz, 1H, CH₂Ph), 4.62 (dd, J=12.6, 6.1 Hz, 1H, H-6b), 4.53 (t, J=6.1 Hz, 1H, H-4), 4.28 (dd, J=4.4, 2.9 Hz, 1H, H-2), 4.13 (dd, J=6.1, 2.9 Hz, 1H, H-3), 3.40 (s, 3H, CH₃), 2.95 (s, 3H, CH₃); ¹³C NMR (100 MHz, CDCl₃) δ 166.0 (C), 137.3 (C), 129.7 (CH), 128.5 (CH), 128.4 (CH), 127.9 (CH), 127.8 (CH), 102.3 (CH), 82.9 (CH), 77.3 (CH), 76.7 (CH), 76.0 (CH), 71.9 (CH₂), 64.1 (CH₂), 56.1 (CH₃), 38.8 (CH₃); HRMS (FAB, MH⁺) calcd for C₂₂H₂₇O₉S 467.1378, found 467.1376.

Synthesis of Compound 29: Sodium hydride (309 mg, 7.70 mmol) was added to a solution of the 2-alcohol 28 (3.00 g, 6.40 mmol) and benzyl bromide (0.9 mL, 7.70 mmol) in DMF (30 mL) at 0° C. under nitrogen. After stirring for 10 min, the mixture was diluteted with EtOAc (50 mL), and the organic layer was consecutively washed with water (3×30 mL) and brine, dried over MgSO₄, filtered, and concentrated in vacuo. The residue was purified by flash column chromatography (EtOAc/Hex=1/3) on silica gel to give 29 (3.20 g, 94%). ¹H NMR (400 MHz, CDCl₃) [symbol] 8.07 (dd, J=5, 8, 0.8 Hz, 2H, BzH), 7.58-7.52 (m, 1H, BzH), 7.46-7.40 (m, 2H, BzH), 7.39-7.22 (m, 10H, ArH), 5.34 (td, J=4.7, 1.7 Hz, 1H, H-5), 4.89 (d, J=3.6 Hz, 1H, H-1), 4.76 (dd, J=10.0, 1.7 Hz, 1H, H-6a), 4.67-4.52 (m, 6H, CH₂Ph, H-4, H-6b), 4.36 (dd, J=5.1, 3.6 Hz, 1H, H-3), 4.02 (t, J=3.6 Hz, 1H, H-2), 3.38 (s, 3H, CH₃), 2.93 (s, 3H, CH₃); ¹³C NMR (100 MHz, CDCl₃) δ 166.1 (C), 137.43 (C), 137.38 (C), 133.3 (CH), 129.7 (CH), 129.6 (CH), 128.5 (CH), 128.4 (CH), 128.3 (CH), 128.2 (CH), 128.1 (CH), 127.8 (CH), 101.4 (CH), 83.5 (CH), 81.2 (CH), 77.9 (CH), 76.3 (CH), 72.8 (CH₂), 72.4 (CH₂), 63.9 (CH₂), 55.7 (CH₃), 38.8 (CH₃); HRMS (FAB, MNa⁺) calcd for C₂₉H₃₂O₉SNa 579.1670, found 579.1665.

Synthesis of Compound 30: Following the procedure for the preparation of compound 26 furnished the product 30 (45%) as a white solid. ¹H NMR (400 MHz, CDCl₃) [symbol] 7.36-7.24 (m, 10H, ArH), 5.32 (d, J=1.6 Hz, 1H, H-1), 4.91 (d, J=11.7 Hz, 1H, CH₂Ph), 4.68 (s, 2H, CH₂Ph), 4.67 (d, J=11.7 Hz, 1H, CH₂Ph), 4.38 (t, J=4.8 Hz, 1H, H-5), 4.04 (d, J=7.6 Hz, 1H, H-6a), 3.82-3.78 (m, 1H, H-4), 3.68 (dd, J=7.6, 4.8 Hz, 1H, H-6b), 3.59 (t, J=7.8 Hz, 1H, H-3), 3.48 (dd, J=7.8, 1.6 Hz, 1H, H-2), 2.00 (d, J=3.1 Hz, 1H, OH-4); ¹³C NMR (100 MHz, CDCl₃) δ 138.5 (C), 137.8 (C), 128.6 (CH), 128.5 (CH), 128.0 (CH), 127.94 (CH), 127.88 (CH), 99.6 (CH), 82.6 (CH), 82.2 (CH), 75.0 (CH₂), 74.9 (CH), 72.8 (CH₂), 71.2 (CH), 65.3 (CH₂). Anal. Calcd for C₂₀H₂₂O₅: C, 70.16; H, 6.48. Found: C, 69.69; H, 6.42.

### Preparation of 48 Disaccharide Building Blocks for Making HP/HS

48 disaccharide building blocks were prepared from eight monosaccharide units as shown in Scheme 5 below.

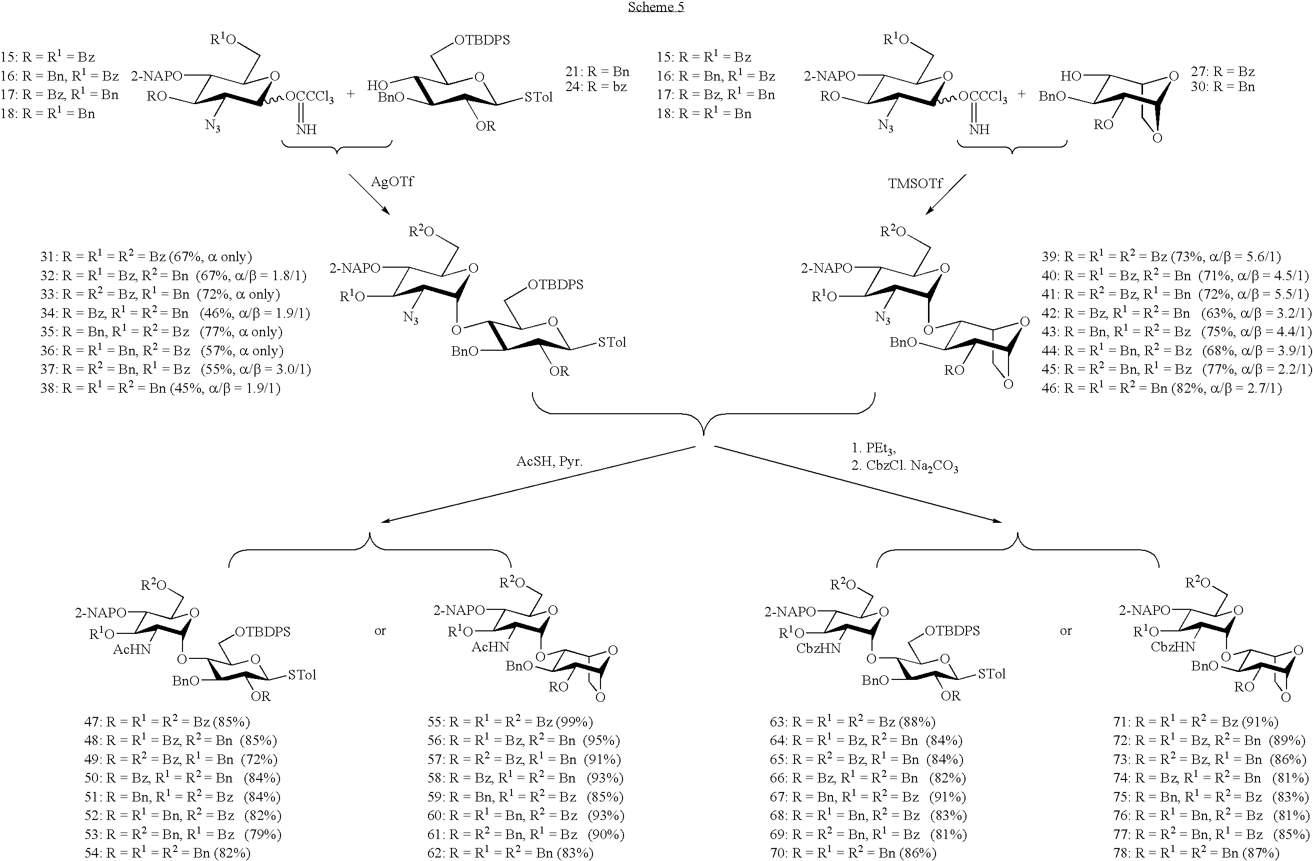

Synthesis of the Disaccharides 31-38. A solution of the trichloroacetimidate 15, 16, 17 or 18 (1.0 equiv), the thioglycoside 21 or 24 (1.5 equiv), and freshly dried 3 Å molecular sieves (1 mg per 1 mg of a mixture of the trichloroacetimidate and the thioglycoside) in dichloromethane (1 mL per 100 mg of a mixture of the trichloroacetimidate and the thioglycoside) was stirred at room temperature for 1 h under nitrogen. The mixture was cooled down to −40° C., and sliver trifluoromethanesulfonate (AgOTf, 5.0 equiv) was added to the reaction solution. After stirring at the same temperature for 2 h, the reaction was quenched by saturated NaHCO₃ (aq), the whole mixture was filtered through celite, and the filtrate was extracted with ethyl acetate (three times). The combined organic layers were washed with brine, dried over magnesium sulfate, filtered, and concentrated in vacuo. The residue was purified by flash column chromatography on silica gel to give the expected disaccharide. The yields of compounds 31-38 are summarized in Scheme 5. 31: ¹H NMR (500 MHz, CDCl₃) δ 8.06 (d, J=8.0 Hz, 2H, ArH), 8.02 (d, J=8.1 Hz, 2H, ArH), 7.84 (d, J=8.0 Hz, 2H, ArH), 7.74-7.71 (m, 2H, ArH), 7.67-7.64 (m, 3H, ArH), 7.60-7.34 (m, 14H, ArH), 7.31-7.28 (m, 5H, ArH), 7.22-7.18 (m, 4H, ArH), 7.11 (s, 5H, ArH), 6.98 (d, J=7.9 Hz, 2H, ArH), 5.74 (dd, J=10.8, 8.8 Hz, 1H, H-3′), 5.72 (d, J=3.9 Hz, 1H, H-1′), 5.35 (dd, J=10.2, 9.0 Hz, 1H, H-2), 4.85 (d, J=10.2 Hz, 1H, H-1), 4.70, 4.69 (ABq, J=10.4 Hz, 2H, CH₂Ar), 4.62 (s, 2H, CH₂Ar), 4.08-4.03 (m, 4H, H-3, H-6a, H-6′a, H-6′b), 3.96 (t, J=9.1 Hz, 1H, H-4), 3.94 (dd, J=11.0, 5.7 Hz, 1H, H-6b), 3.78 (dt, J=10.1, 2.7 Hz, 1H, H-5′), 3.71 (dd, J=10.1, 8.8 Hz, 1H, H-4′), 3.66 (ddd, J=9.1, 5.7, 2.0 Hz, 1H, H-5), 3.16 (dd, J=10.8, 3.9 Hz, 1H, H-2′), 2.27 (s, 3H, CH₃), 1.07 (s, 9H, t-Bu); ¹³C NMR (125 MHz, CDCl₃) δ 165.9 (C), 165.5 (C), 165.3 (C), 137.6 (C), 137.3 (C), 135.8 (CH), 135.5 (CH), 134.4 (C), 133.4 (CH), 133.30 (CH), 133.28 (C), 133.02 (CH), 132.96 (C), 132.9 (C), 132.7 (C), 132.0 (CH), 130.4 (C), 129.9 (CH), 129.8 (CH), 129.7 (CH), 129.5 (CH), 129.3 (C), 128.5 (CH), 128.3 (CH), 128.2 (CH), 127.8 (CH), 127.63 (CH), 127.57 (CH), 127.2 (CH), 126.04 (CH), 125.99 (CH), 97.9 (CH), 81.4 (CH), 85.0 (CH), 79.5 (CH), 75.7 (CH), 75.0 (CH₂), 74.3 (CH₂), 74.0 (CH), 72.9 (CH), 72.6 (CH), 69.8 (CH), 64.0 (CH₂), 62.7 (CH₂), 61.3 (CH), 26.9 (1-Bu), 21.1 (CH₃), 19.3 (C); HRMS (FAB, MNa⁺) calcd for C₇₄H₇₁N₃O₁₂SSiNa 1276.4425, found 1276.4434. 32: ¹H NMR (400 MHz, CDCl₃) δ 8.10 (dd, J=8.4, 1.2 Hz, 2H, ArH), 7.95 (dd, J=8.4, 1.1 Hz, 2H, ArH), 7.71-7.40 (m, 12H, ArH), 7.37-7.07 (m, 23H, ArH), 7.01 (d, J=8.0 Hz, 2H, ArH), 5.80 (d, J=3.9 Hz, 1H, H-1′), 5.66 (dd, J=10.6, 9.3 Hz, 1H, H-3′), 5.39 (dd, J=10.1, 8.9 Hz, 1H, H-2), 4.87 (d, J=10.1 Hz, 1H, H-1), 4.71 (s, 2H, CH₂Ar), 4.55-4.47 (m, 3H, CH₂Ar), 4.19 (d, J=12.1 Hz, 1H, CH₂Ar), 4.10-4.03 (m, 3H, H-3, H-4, H-6a), 3.97 (dd, J=11.3, 5.1 Hz, 1H, H-6b), 3.88 (t, J=9.3 Hz, 1H, H-4′), 3.64-3.59 (m, 2H, H-5, H-5′), 3.22 (dd, J=11.0, 1.6 Hz, 1H, H-6′a), 3.15 (dd, J=10.6, 3.9 Hz, 1H, H-2′), 3.13-3.10 (m, 1H, H-6′b), 2.29 (s, 3H, CH₃), 1.09 (s, 9H, t-Bu); ¹³C NMR (100 MHz, CDCl₃) [symbol] 165.5 (C), 165.3 (C), 137.6 (C), 137.5 (C), 137.3 (C), 135.8 (CH), 135.5 (CH), 135.0 (C), 133.9 (C), 133.3 (CH), 133.2 (CH), 133.0 (C), 132.9 (C), 132.8 (C), 131.8 (CH), 130.5 (C), 129.9 (CH), 129.75 (CH), 129.71 (CH), 129.7 (CH), 129.6 (CH), 129.4 (C), 128.5 (CH), 128.4 (CH), 128.33 (CH), 128.27 (CH), 128.2 (CH), 128.0 (CH), 127.9 (CH), 127.8 (CH), 127.72 (CH), 127.68 (CH), 127.6 (CH), 126.8 (CH), 126.0 (CH), 125.9 (CH), 125.8 (CH), 98.2 (CH), 87.5 (CH), 85.2 (CH), 79.4 (CH), 75.4 (CH), 74.7 (CH₂), 74.5 (CH₂), 73.6 (CH₂), 73.2 (CH), 73.1 (CH), 72.5 (CH), 71.2 (CH), 67.3 (CH₂), 63.7 (CH₂), 61.3 (CH), 26.9 (t-Bu), 21.1 (CH₃), 19.4 (C); HRMS (FAB, MNa⁺) calcd for C₇₄H₇₃N₃O₁₁SSiNa 1262.4633, found 1262.4597. 33: ¹H NMR (500 MHz, CDCl₃) δ 8.07 (dd, J=8.5, 1.2 Hz, 2H, ArH), 7.83-7.78 (m, 4H, ArH), 7.76-7.64 (m, 6H, ArH), 7.57 (tt, J=7.3, 1.2 Hz, 1H, ArH), 7.50-7.42 (m, 5H, ArH), 7.40-7.21 (m, 16H, ArH), 7.19-7.11 (m, 5H, ArH), 6.98 (d, J=8.0 Hz, 2H, ArH), 5.58 (d, J=3.9 Hz, 1H, H-1′), 5.34 (dd, J=10.1, 8.7 Hz, 1H, H-2), 4.95 (d, J=10.9 Hz, 1H, CH₂Ar), 4.89, 4.87 (ABq, J=10.6 Hz, 2H, CH₂Ar), 4.86 (d, J=10.1 Hz, 1H, H-1), 4.77, 4.70 (ABq, J=10.3 Hz, 2H, CH₂Ar), 4.68 (d, J=10.9 Hz, 1H, CH₂Ar), 4.11-4.08 (m, 2H, H-6′a, H-6′b), 4.04 (dd, J=11.1, 1.8 Hz, 1H, H-6a), 4.03 (dd, J=9.0, 8.7 Hz, 1H, H-3), 3.93 (dd, J=11.1, 6.0 Hz, 1H, H-6b), 3.92 (t, J=9.0 Hz, 1H, H-4), 3.86 (dd, J=10.3, 8.9 Hz, 1H, H-3′), 3.72-3.65 (m, 2H, H-5, H-5′), 3.58 (dd, J=9.6, 8.9 Hz, 1H, H-4′), 3.24 (dd, J=10.3, 3.9 Hz, 1H, H-2′), 2.27 (s, 3H, CH₃), 1.07 (s, 9H, t-Bu); ¹³C NMR (125 MHz, CDCl₃) δ 165.9 (C), 165.2 (C), 137.6 (C), 137.4 (C), 135.7 (CH), 135.6 (CH), 134.9 (C), 133.3 (CH), 133.2 (C), 133.14 (C), 133.05 (C), 133.0 (CH), 132.0 (CH), 130.4 (C), 129.8 (CH), 129.71 (CH), 129.66 (CH), 129.6 (C), 129.5 (CH), 128.6 (CH), 128.5 (CH), 128.31 (CH), 128.27 (CH), 128.0 (CH), 127.85 (CH), 127.79 (CH), 127.77 (CH), 127.70 (CH), 127.67 (CH), 127.6 (CH), 126.9 (CH), 126.2 (CH), 126.1 (CH), 125.9 (CH), 97.5 (CH), 87.3 (CH), 84.8 (CH), 80.1 (CH), 79.7 (CH), 78.4 (CH), 75.6 (CH₂), 75.3 (CH₂), 74.3 (CH₂), 73.7 (CH), 72.9 (CH), 70.2 (CH), 64.1 (CH₂), 63.3 (CH), 62.8 (CH₂), 26.9 (t-Bu), 21.1 (CH₃), 19.3 (C); HRMS (FAB, MNa⁺) calcd for C₇₄H₇₃N₃O₁₁SSiNa 1262.4633, found 1262.4664. 34: ¹H NMR (400 MHz, CDCl₃) δ 8.07 (dd, J=8.5, 1.2 Hz, 2H, ArH), 7.85-7.81 (m, 1H, ArH), 7.79-7.72 (m, 2H, ArH), 7.70-7.63 (m, 4H, ArH), 7.57 (tt, J=7.4, 1.3 Hz, 1H, ArH), 7.52-7.42 (m, 5H, ArH), 7.37-7.10 (m, 24H, ArH), 6.98 (d, J=7.9 Hz, 2H, ArH), 5.62 (d, J=4.0 Hz, 1H, H-1′), 5.35 (dd, J=10.1, 8.8 Hz, 1H, H-2), 4.86, 4.81 (ABq, J=10.7 Hz, 2H, CH₂Ar), 4.85 (d, J=10.1 Hz, 1H, H-1), 4.83 (d, J=11.0 Hz, 1H, CH₂Ar), 4.75, 4.69 (ABq, J=10.1 Hz, 2H, CH₂Ar), 4.53 (d, J=11.0 Hz, 1H, CH₂Ar), 4.45 (d, J=12.1 Hz, 1H, CH₂Ar), 4.16 (d, J=12.1 Hz, 1H, CH₂Ar), 4.02 (t, J=8.8 Hz, 1H, H-3), 4.00-3.91 (m, 2H, H-4, H-6a), 3.90 (dd, J=11.2, 5.8 Hz, 1H, H-6b), 3.77 (dd, J=10.2, 9.0 Hz, 1H, H-3′), 3.69 (dd, J=9.8, 9.0 Hz, 1H, H-4′), 3.64-3.59 (m, 1H, H-5), 3.50-3.45 (m, 1H, H-5′), 3.27 (dd, J=10.9, 2.3 Hz, 1H, H-6′a), 3.21 (dd, J=10.2, 4.0 Hz, 1H, H-2′), 3.10 (dd, J=10.9, 1.6 Hz, 1H, H-6′b), 2.27 (s, 3H, CH₃), 1.06 (s, 9H, t-Bu); ¹³C NMR (100 MHz, CDCl₃) [symbol] 165.2 (C), 137.9 (C), 137.7 (C), 137.5 (C), 137.4 (C), 135.8 (CH), 135.6 (C), 135.5 (CH), 133.7 (C), 133.3 (CH), 133.2 (C), 133.1 (C), 133.0 (C), 131.8 (CH), 130.1 (C), 129.84 (CH), 129.78 (C), 129.65 (CH), 129.59 (CH), 128.5 (CH), 128.4 (CH), 128.33 (CH), 128.28 (CH), 127.98 (CH), 127.96 (CH), 127.84 (CH), 127.80 (CH), 127.71 (CH), 127.65 (CH), 126.4 (CH), 126.1 (CH), 125.93 (CH), 125.91 (CH), 97.8 (CH), 87.3 (CH), 85.0 (CH), 79.9 (CH), 79.6 (CH), 77.8 (CH), 75.3 (CH₂), 74.9 (CH₂), 74.5 (CH₂), 73.5 (CH₂), 73.3 (CH), 73.1 (CH), 71.6 (CH), 67.4 (CH₂), 63.9 (CH₂), 63.0 (CH), 26.9 (t-Bu), 21.1 (CH₃), 19.3 (C); HRMS (FAB, MNa⁺) calcd for C₇₄H₇₅N₃O₁₀SSiNa 1248.4840, found 1248.4855. 35: ¹H NMR (500 MHz, CDCl₃) δ 8.01 (d, J=7.2 Hz, 2H, ArH), 7.85 (d, J=7.2 Hz, 2H, ArH), 7.74-7.70 (m, 2H, ArH), 7.67-7.63 (m, 3H, ArH), 7.60-7.44 (m, 7H, ArH), 7.43-7.16 (m, 23H, ArH), 7.03 (d, J=8.0 Hz, 2H, ArH), 5.78 (d, J=3.9 Hz, 1H, H-1′), 5.72 (dd, J=10.7, 8.8 Hz, 1H, H-3′), 5.06 (d, J=10.9 Hz, 1H, CH₂Ar), 4.99 (d, J=10.2 Hz, 1H, CH₂Ar), 4.81 (d, J=10.9 Hz, 1H, CH₂Ar), 4.73 (d, J=9.9 Hz, 1H, H-1), 4.65 (d, J=10.2 Hz, 1H, CH₂Ar), 4.62 (s, 2H, CH₂Ar), 4.09-4.07 (m, 2H, H-6′a, H-6′b), 4.03 (dd, J=11.1, 1.5 Hz, 1H, H-6a), 3.91 (dd, J=11.1, 5.9 Hz, 1H, H-6b), 3.87-3.83 (m, 2H, H-3, H-4), 3.80-3.77 (m, 1H, H-5′), 3.72 (dd, J=9.8, 8.8 Hz, 1H, H-4′), 3.55-3.50 (m, 2H, H-2, H-5), 3.15 (dd, J=10.7, 3.9 Hz, 1H, H-2′), 2.31 (s, 3H, CH₃), 1.09 (s, 9H, t-Bu); ¹³C NMR (125 MHz, CDCl₃) δ 165.9 (C), 165.4 (C), 138.3 (C), 137.8 (C), 137.3 (C), 136.1 (CH), 135.8 (CH), 135.6 (CH), 134.4 (C), 133.43 (C), 133.38 (CH), 133.0 (CH), 132.8 (C), 131.5 (CH), 130.9 (C), 129.8 (CH), 129.7 (CH), 129.6 (CH), 129.5 (CH), 129.4 (C), 128.45 (CH), 128.39 (CH), 128.3 (CH), 128.2 (CH), 128.0 (CH), 127.8 (CH), 127.6 (CH), 127.4 (CH), 127.3 (CH), 127.2 (CH), 126.04 (CH), 125.96 (CH), 97.9 (CH), 88.6 (CH), 86.6 (CH), 81.7 (CH), 79.2 (CH), 75.7 (CH), 75.0 (CH₂), 74.9 (CH₂), 74.7 (CH₂), 73.8 (CH), 72.6 (CH), 69.8 (CH), 64.1 (CH₂), 62.8 (CH₂), 61.3 (CH), 26.9 (t-Bu), 21.1 (CH₃), 19.3 (C); HRMS (FAB, MH⁺) calcd for C₇₄H₇₄N₃O₁₁SSi 1240.4813, found 1240.4812. 36: ¹H NMR (500 MHz, CDCl₃) δ 7.82 (dd, J=8.4, 1.2 Hz, 2H, ArH), 7.79-7.76 (m, 2H, ArH), 7.73-7.62 (m, 6H, ArH), 7.49-7.42 (m, 5H, ArH), 7.41-7.17 (m, 24H, ArH), 7.01 (d, J=8.1 Hz, 2H, ArH), 5.63 (d, J=3.9 Hz, 1H, H-1′), 5.04, 4.89 (ABq, J=11.1 Hz, 2H, CH₂Ar), 4.96 (d, J=10.3 Hz, 1H, CH₂Ar), 4.93 (d, J=11.1 Hz, 1H, CH₂Ar), 4.85 (s, 2H, CH₂Ar), 4.71 (d, J=9.9 Hz, 1H, H-1), 4.67 (d, J=11.1 Hz, 1H, CH₂Ar), 4.65 (d, J=10.3 Hz, 1H, CH₂Ar), 4.13-4.07 (m, 2H, H-6′a, H-6′b), 4.00 (dd, J=11.1, 1.9 Hz, 1H, H-6a), 3.87 (dd, J=11.1, 6.1 Hz, 1H, H-6b), 3.83 (dd, J=10.3, 8.8 Hz, 1H, H-3), 3.81-3.77 (m, 2H, H-4, H-3′), 3.70-3.68 (m, 1H, H-5′), 3.58 (dd, J=9.9, 8.9 Hz, 1H, H-4′), 3.54-3.48 (m, 2H, H-2, H-5), 3.22 (dd, J=10.4, 3.9 Hz, 1H, H-2′), 2.29 (s, 3H, CH₃), 1.06 (s, 9H, t-Bu); ¹³C NMR (125 MHz, CDCl₃) δ 165.9 (C), [symbol] (C), 137.9 (C), 137.6 (C), 137.3 (C), 135.8 (CH), 135.6 (CH), 134.9 (C), 133.3 (C), 133.1 (C), 133.04 (C), 132.96 (CH), 131.6 (CH), 130.9 (C), 129.74 (CH), 129.66 (CH), 129.5 (CH), 128.5 (CH), 128.4 (CH), 128.3 (CH), 128.2 (CH), 128.1 (CH), 128.0 (CH), 127.8 (CH), 127.73 (CH), 127.69 (CH), 127.4 (CH), 127.1 (CH), 126.8 (CH), 126.2 (CH), 126.0 (CH), 125.9 (CH), 97.5 (CH), 88.5 (CH), 86.6 (CH), 81.5 (CH), 80.0 (CH), 79.4 (CH), 77.9 (CH), 75.6 (CH₂), 75.2 (CH₂×2), 74.7 (CH₂), 73.9 (CH), 70.1 (CH), 64.2 (CH₂), 63.3 (CH), 62.8 (CH₂), 26.9 (t-Bu), 21.1 (CH₃), 19.3 (C); HRMS (FAB, MNa⁺) calcd for C₇₄H₇₅N₃O₁₀SSiNa 1248.4840, found 1248.4852. 37: ¹H NMR (500 MHz, CDCl₃) δ 7.91 (dd, J=8.4, 1.0 Hz, 2H, ArH), 7.69-7.57 (m, 6H, ArH), 7.52 (d, J=8.5 Hz, 1H, ArH), 7.50-7.43 (m, 3H, ArH), 7.41-7.19 (m, 23H, ArH), 7.18-7.12 (m, 3H, ArH), 7.08-7.02 (m, 3H, ArH), 5.84 (d, J=4.0 Hz, 1H, H-1′), 5.62 (dd, J=10.7, 9.2 Hz, 1H, H-3′), 5.06 (d, J=10.7 Hz, 1H, CH₂Ar), 5.01 (d, J=10.3 Hz, 1H, CH₂Ar), 4.79 (d, J=10.7 Hz, 1H, CH₂Ar), 4.73 (d, J=9.9 Hz, 1H, H-1), 4.69 (d, J=10.3 Hz, 1H, CH₂Ar), 4.52-4.43 (m, 3H, CH₂Ar), 4.17 (d, J=12.0 Hz, 1H, CH₂Ar), 4.00 (dd, J=11.2, 2.0 Hz, 1H, H-6a), 3.93-3.90 (m, 2H, H-4, H-6b), 3.86 (t, J=8.7 Hz, 1H, H-3), 3.85 (t, J=9.2 Hz, 1H, H-4′), 3.61-3.56 (m, 1H, H-5′), 3.52 (dd, J=9.9, 8.7 Hz, 1H, H-2), 3.49 (ddd, J=9.6, 5.2, 2.0 Hz, 1H, H-5), 3.20 (dd, J=10.9, 2.0 Hz, 1H, H-6′a), 3.12 (dd, J=10.7, 4.0 Hz, 1H, H-2′), 3.10 (dd, J=10.9, 1.6 Hz, 1H, H-6′b), 2.30 (s, 3H, CH₃), 1.06 (s, 9H, t-Bu); ¹³C NMR (100 MHz, CDCl₃) [symbol] 165.5 (C), 138.2 (C), 137.9 (C), 137.6 (C), 137.2 (C), 135.8 (CH), 135.5 (CH), 135.1 (C), 134.0 (C), 133.2 (CH), 133.0 (C), 132.9 (C), 131.4 (CH), 131.1 (C), 129.8 (CH), 129.7 (CH), 129.52 (CH), 129.47 (C), 128.41 (CH), 128.39 (CH), 128.3 (CH), 128.2 (CH), 128.1 (CH), 127.93 (CH), 127.86 (CH), 127.83 (CH), 127.76 (CH), 127.74 (CH), 127.69 (CH), 127.6 (CH), 127.53 (CH), 127.47 (CH), 126.7 (CH), 126.0 (CH), 125.9 (CH), 125.8 (CH), 98.2 (CH), 88.7 (CH), 86.8 (CH), 81.8 (CH), 79.1 (CH), 75.4 (CH), 75.2 (CH₂), 74.8 (CH₂), 74.6 (CH₂), 73.6 (CH₂), 73.3 (CH), 72.5 (CH), 71.0 (CH), 67.4 (CH₂), 63.7 (CH₂), 61.2 (CH), 27.0 (t-Bu), 21.1 (CH₃), 19.4 (C); HRMS (FAB, N4Na⁺) calcd for C₇₄H₇₅N₃O₁₀SSiNa 1248.4840, found 1248.4852. 38: ¹H NMR (400 MHz, CDCl₃) δ 7.84-7.71 (m, 3H, ArH), 7.69-7.63 (m, 4H, ArH), 7.52-7.43 (m, 5H, ArH), 7.37-7.15 (m, 27H, ArH), 7.01 (d, J=8.0 Hz, 2H, ArH), 5.67 (d, J=3.9 Hz, 1H, H-1′), 5.04, 4.88 (ABq, J=10.9 Hz, 2H, CH₂Ar), 4.99 (d, J=10.3 Hz, 1H, CH₂Ar), 4.85-4.78 (m, 3H, CH₂Ar), 4.71 (d, J=9.6 Hz, 1H, H-1), 4.68 (d, J=10.3 Hz, 1H, CH₂Ar), 4.54 (d, J=11.2 Hz, 1H, CH₂Ar), 4.44, 4.18 (ABq, J=12.1 Hz, 2H, CH₂Ar), 3.99 (dd, J=11.3, 1.9 Hz, 1H, H-6a), 3.88 (dd, J=11.3, 5.9 Hz, 1H, H-6b), 3.86-3.73 (m, 3H, H-4, H-3′, H-4′), 3.67 (t, J=9.3 Hz, 1H, H-3), 3.54-3.48 (m, 3H, H-2, H-5, H-5′), 3.31 (dd, J=10.8, 2.2 Hz, 1H, H-6′a), 3.19 (dd, J=10.2, 3.9 Hz, 1H, H-2′), 3.15 (d, J=10.8 Hz, 1H, H-6′b), 2.29 (s, 3H, CH₃), 1.05 (s, 9H, t-Bu); ¹³C NMR (100 MHz, CDCl₃) [symbol] 138.4 (C), 138.1 (C), 138.0 (C), 137.9 (C), 135.8 (CH), 135.7 (C), 135.6 (CH), 133.9 (C), 133.32 (C), 133.29 (C), 133.0 (C), 131.6 (CH), 131.1 (C), 129.7 (CH), 129.62 (CH), 129.57 (CH), 128.42 (CH), 128.40 (CH), 128.3 (CH), 128.1 (CH), 127.95 (CH), 127.86 (CH), 127.81 (CH), 127.76 (CH), 127.72 (CH), 127.70 (CH), 127.5 (CH), 127.3 (CH), 126.4 (CH), 126.1 (CH), 125.9 (CH), 97.9 (CH), 88.7 (CH), 86.8 (CH), 81.8 (CH), 79.9 (CH), 79.5 (CH), 78.0 (CH), 75.3 (CH₂), 75.2 (CH₂), 74.9 (CH₂), 74.8 (CH₂), 73.7 (CH), 73.5 (CH₂), 71.6 (CH), 67.8 (CH₂), 64.2 (CH₂), 63.2 (CH), 27.0 (t-Bu), 21.1 (CH₃), 19.4 (C); HRMS (FAB, MNa⁺) calcd for C₇₄H₇₇N₃O₉SSiNa 1234.5048, found 1234.5043.

Synthesis of Compounds 39-46: A solution of the trichloroacetimidate 15, 16, 17 or 18 (1.1 equiv), the 4-alcohol 27 or 30 (1.0 equiv), and freshly dried 3 Å molecular sieves (1 mg per 1 mg of a mixture of the trichloroacetimidate and the 4-alcohol) in dichloromethane (1 mL per 100 mg of a mixture of the trichloroacetimidate and the 4-alcohol) was stirred at room temperature for 1 h under nitrogen. The reaction flask was cooled down to −78° C., trimethylsilyl trifluoromethanesulfonate (TMSOTf, 0.2 equiv) was added to the solution, and the mixture was gradually warmed up to room temperature. After stirring for 1.5 h, triethylamine (0.3 equiv) was added to quench the reaction, and the mixture was filtered through celite, and the solid was washed by dichloromethane. The filtrate was concentrated in vacuo to furnish a residue, which was purified by flash column chromatography to give the expected disaccharide. The yields of compounds 39-46 are summarized in Scheme 5. 39: ¹H NMR (500 MHz, CDCl₃) δ 8.05 (d, J=7.3 Hz, 4H, BzH), 7.98 (d, J=7.3 Hz, 2H, BzH), 7.67-7.55 (m, 6H, ArH), 7.51 (s, 1H, ArH), 7.45-7.37 (m, 8H, ArH), 7.25-7.14 (m, 6H, ArH), 5.88 (dd, J=10.7, 9.0 Hz, 1H, H-3′), 5.53 (d, J=1.6 Hz, 1H, H-1), 5.46 (d, J=3.7 Hz, 1H, H-1′), 5.06 (dd, J=8.1, 1.6 Hz, 1H, H-2), 4.79, 4.75 (ABq, J=10.8 Hz, 2H, CH₂Ar), 4.74 (s, 2H, CH₂Ar), 4.66-4.63 (m, 2H, H-6′a, H-5), 4.57 (dd, J=12.0, 5.4 Hz, 1H, H-6′b), 4.25 (d, J=7.9 Hz, 1H, H-6a), 4.17 (t, J=8.1 Hz, 1H, H-3), 4.14-4.10 (m, 2H, H-4, H-5′), 3.83-3.80 (m, 1H, H-6b), 3.82 (t, J=9.0 Hz, 1H, H-4′), 3.37 (dd, J=10.7, 3.7 Hz, 1H, H-2′); ¹³C NMR (125 MHz, CDCl₃) δ 166.1 (C), 165.7 (C), 165.4 (C), 133.9 (C), 133.5 (CH), 133.4 (CH), 133.3 (CH), 133.03 (C), 132.96 (C), 129.84 (CH), 129.79 (CH), 129.6 (CH), 129.5 (C), 129.3 (C), 129.1 (C), 128.52 (CH), 128.46 (CH), 128.3 (CH), 127.8 (CH), 127.6 (CH), 127.3 (CH), 126.1 (CH), 125.8 (CH), 99.4 (CH), 99.3 (CH), 79.8 (CH), 77.5 (CH), 77.2 (CH), 76.3 (CH), 75.3 (CH₂), 74.9 (CH₂), 74.3 (CH), 72.4 (CH), 69.8 (CH), 65.8 (CH₂), 63.2 (CH₂), 61.4 (CH); HRMS (FAB, MH⁺) calcd for C₅₁H₄₆N₃O₁₂ 892.3081, found 892.3080. 40: ¹H NMR (400 MHz, CDCl₃) δ 8.07 (dd, J=8.5, 1.3 Hz, 2H, BzH), 7.98 (dd, J=8.4, 1.2 Hz, 2H, BzH), 7.68-7.66 (m, 1H, ArH), 7.61-7.59 (m, 1H, ArH), 7.58-7.51 (m, 3H, ArH), 7.45-7.28 (m, 12H, ArH), 7.23-7.09 (m, 6H, ArH), 5.79 (dd, J=10.8, 8.8 Hz, 1H, H-3′), 5.53 (d, J=1.7 Hz, 1H, H-1), 5.47 (d, J=3.8 Hz, 1H, H-1′), 5.08 (dd, J=7.9, 1.7 Hz, 1H, H-2), 4.80, 4.75 (ABq, J=10.8 Hz, 2H, CH₂Ar), 4.66-4.51 (m, 5H, H-5, CH₂Ar), 4.22 (d, J=7.9 Hz, 1H, H-6′a), 4.17-4.08 (m, 2H, H-3, H-4), 3.93-3.87 (m, 1H, H-4′), 3.87-3.85 (m, 1H, H-5′), 3.83-3.69 (m, 3H, H-6a, H-6b, H-6′b), 3.37 (dd, J=10.8, 3.8 Hz, 1H, H-2′); ¹³C NMR (100 MHZ, CDCl₃) δ 165.7 (C), 165.5 (C), 137.8 (C), 137.5 (C), 134.5 (C), 133.41 (CH), 133.36 (CH), 133.1 (C), 133.0 (C), 129.9 (CH), 129.8 (CH), 129.4 (C), 129.3 (C), 128.54 (CH), 128.47 (CH), 128.4 (CH), 128.3 (CH), 128.2 (CH), 128.04 (CH), 127.99 (CH), 127.8 (CH), 127.7 (CH), 127.6 (CH), 127.0 (CH), 126.04 (CH), 125.97 (CH), 125.9 (CH), 99.7 (CH), 99.3 (CH), 79.8 (CH), 77.8 (CH), 77.3 (CH), 76.0 (CH), 75.1 (CH₂), 74.9 (CH₂), 74.3 (CH), 73.8 (CH₂), 72.4 (CH), 71.3 (CH), 68.0 (CH₂), 65.9 (CH₂), 61.5 (CH); HRMS (FAB, MH⁺) calcd for C₅₁H₄₈N₃O₁₁, 878.3289, found 878.3291. 41: ¹H NMR (400 MHz, CDCl₃) δ 8.04 (d, J=8.4 Hz, 2H, BzH), 7.95 (d, J=8.4 Hz, 2H, BzH), 7.81-7.73 (m, 3H, ArH), 7.69 (s, 1H, ArH), 7.60-7.52 (m, 2H, ArH), 7.46-7.32 (m, 13H, ArH), 7.25-7.17 (m, 4H, ArH), 5.53 (d, J=1.6 Hz, 1H, H-1), 5.31 (d, J=3.7 Hz, 1H, H-1′), 5.06 (dd, J=7.7, 1.6 Hz, 1H, H-2), 5.04 (d, J=10.9 Hz, 1H, CH₂Ar), 4.99 (d, J=10.6 Hz, 1H, CH₂Ar), 4.95 (d, J=10.6 Hz, 1H, CH₂Ar), 4.88 (d, J=10.9 Hz, 1H, CH₂Ar), 4.82 (d, J=10.9 Hz, 1H, CH₂Ar), 4.76 (d, J=10.9 Hz, 1H, CH₂Ar), 4.65-4.60 (m, 2H, H-5, H-6′a), 4.47 (dd, J=12.0, 5.1 Hz, 1H, H-6′b), 4.19 (d, J=7.8 Hz, 1H, H-6a), 4.11 (t, J=7.7 Hz, 1H, H-3), 4.09-4.03 (m, 2H, H-4, H-3′), 3.96 (ddd, J=9.6, 5.1, 1.7 Hz, 1H, H-5′), 3.78 (dd, J=7.8, 5.2 Hz, 1H, H-6b), 3.68 (t, J=9.6 Hz, 1H, H-4′), 3.45 (dd, J=10.4, 3.7 Hz, 1H, H-2′); ¹³C NMR (100 MHz, CDCl₃) δ 166.08 (C), 165.67 (C), 137.83 (C), 137.47 (C), 134.45 (C), 133.37 (CH), 133.26 (CH), 133.06 (C), 133.13 (C), 129.84 (CH), 129.51 (CH), 129.46 (C), 129.37 (C), 128.60 (CH), 128.54 (CH), 128.50 (CH), 128.44 (CH), 128.32 (CH), 128.10 (CH), 127.89 (CH), 127.76 (CH), 127.68 (CH), 127.14 (CH), 126.25 (CH), 126.18 (CH), 125.80 (CH), 99.47 (CH), 99.28 (CH), 80.10 (CH), 79.42 (CH), 78.58 (CH), 78.16 (CH), 77.04 (CH), 75.65 (CH₂), 75.57 (CH₂), 74.92 (CH₂), 74.26 (CH), 70.10 (CH), 65.65 (CH₂), 63.51 (CH), 63.21 (CH₂); HRMS (MALDI, MNa⁺) calcd for C₅₁H₄₇N₃O₁₁Na 900.3108, found 900.3110. 42: ¹H NMR (500 MHz, CDCl₃) δ 8.04 (dd, J=8.4, 1.2 Hz, 2H, BzH), 7.83-7.81 (m, 1H, ArH), 7.78-7.74 (m, 2H, ArH), 7.59-7.55 (m, 2H, ArH), 7.49-7.23 (m, 20H, ArH), 5.53 (d, J=1.6 Hz, 1H, H-1), 5.31 (d, J=3.8 Hz, 1H, H-1′), 5.06 (dd, J=7.7, 1.6 Hz, 1H, H-2), 4.94, 4.66 (ABq, J=11.1 Hz, 2H, CH₂Ar), 4.93 (s, 2H, CH₂Ar), 4.88, 4.75 (ABq, J=1.9 Hz, 2H, CH₂Ar), 4.66 (dd, J=5.2, 4.0 Hz, 1H, H-5), 4.57, 4.48 (ABq, J=12.1 Hz, 2H, CH₂Ar), 4.16 (d, J=7.7 Hz, 1H, H-6a), 4.06 (dd, J=8.1, 7.7 Hz, 1H, H-3), 4.03 (dd, J=8.1, 4.0 Hz, 1H, H-4), 3.98 (dd, J=10.3, 8.1 Hz, 1H, H-3′), 3.76 (dd, J=7.7, 5.2 Hz, 1H, H-6b), 3.72-3.65 (m, 4H, H-4′, H-5′, H-6′a, H-6′b), 3.44 (dd, J=10.3, 3.8 Hz, 1H, H-2′); ¹³C NMR (125 MHz, CDCl₃) δ 165.7 (C), 137.9 (C), 137.7 (C), 137.6 (C), 134.9 (C), 133.4 (C), 133.2 (C), 133.1 (C), 129.9 (CH), 129.4 (C), 128.52 (CH), 128.48 (CH), 128.45 (CH), 128.3 (CH), 128.0 (CH), 127.90 (CH), 127.88 (CH), 127.8 (CH), 127.7 (CH), 126.9 (CH), 126.2 (CH), 126.1 (CH), 126.0 (CH), 99.7 (CH), 99.3 (CH), 80.0 (CH), 79.3 (CH), 79.0 (CH), 78.2 (CH), 77.0 (CH), 75.5 (CH₂), 75.4 (CH₂), 75.0 (CH₂), 74.3 (CH), 73.7 (CH₂), 71.6 (CH), 68.2 (CH₂), 65.8 (CH₂), 63.5 (CH). 43: ¹H NMR (500 MHz, CDCl₃) δ 8.04 (dd, J=8.3, 1.1 Hz, 2H, BzH), 7.97 (dd, J=8.3, 1.1 Hz, 2H, BzH), 7.65 (dd, J=6.0, 3.4 Hz, 1H, ArH), 7.62-7.55 (m, 4H, ArH), 7.50 (s, 1H, ArH), 7.43-7.35 (m, 6H, ArH), 7.33-7.21 (m, 11H, ArH), 5.85 (dd, J=10.7, 9.0 Hz, 1H, H-3′), 5.44 (d, J=3.8 Hz, 1H, H-1′), 5.34 (d, J=1.3 Hz, 1H, H-1), 4.93, 4.82 (ABq, J=10.8 Hz, 2H, CH₂Ar), 4.72 (s, 2H, CH₂Ar), 4.67 (s, 2H, CH₂Ar), 4.62 (dd, J=12.0, 2.2 Hz, 1H, H-6′a), 4.55 (dd, J=12.0, 5.0 Hz, 1H, H-6′b), 4.53 (t, J=4.5 Hz, 1H, H-5), 4.18 (d, J=7.9 Hz, 1H, H-6a), 4.09 (ddd, J=9.8, 5.0, 2.2 Hz, 1H, H-5′), 3.97 (dd, J=8.3, 4.5 Hz, 1H, H-4), 3.94 (dd, J=8.3, 7.4 Hz, 1H, H-3), 3.82 (dd, J=9.8, 9.0 Hz, 1H, H-4′), 3.78 (dd, J=7.9, 4.5 Hz, 1H, H-6b), 3.51 (dd, J=7.4, 1.3 Hz, 1H, H-2), 3.33 (dd, J=10.7, 3.8 Hz, 1H, H-2′); ¹³C NMR (125 MHz, CDCl₃) δ 166.1 (C), 165.4 (C), 138.4 (C), 137.7 (C), 133.9 (C), 133.5 (CH), 133.2 (CH), 133.03 (C), 132.98 (C), 129.8 (CH), 129.6 (CH), 129.2 (C), 128.5 (CH), 128.3 (CH), 128.0 (CH), 127.8 (CH), 127.6 (CH), 127.3 (CH), 126.1 (CH), 125.8 (CH), 99.4 (CH), 99.3 (CH), 83.2 (CH), 81.5 (CH), 77.4 (CH), 76.2 (CH), 75.2 (CH₂×2), 74.3 (CH), 72.8 (CH₂), 72.4 (CH), 69.7 (CH), 65.8 (CH₂), 63.2 (CH₂), 61.4 (CH); HRMS (FAB, M⁺) calcd for C₅₁H₄₇N₃O₁₁ 877.3211, found 877.3199. 44: ¹H NMR (500 MHz, CDCl₃) δ 7.92 (d, J=7.3 Hz, 2H, BzH), 7.80-7.73 (m, 3H, ArH), 7.68 (s, 1H, ArH), 7.53 (t, J=7.5 Hz, 1H, ArH), 7.46-7.27 (m, 20H, ArH), 5.32 (d, J=1.2 Hz, 1H, H-1), 5.26 (d, J=3.8 Hz, 1H, H-1′), 5.03, 4.80 (ABq, J=10.9 Hz, 2H, CH₂Ar), 4.95, 4.94 (ABq, J=10.6 Hz, 2H, CH₂Ar), 4.92, 4.88 (ABq, J=10.7 Hz, 2H, CH₂Ar), 4.69, 4.67 (ABq, J=11.9 Hz, 2H, CH₂Ar), 4.58 (dd, J=12.0, 1.9 Hz, 1H, H-6′a), 4.54 (t, J=4.8 Hz, 1H, H-4), 4.46 (dd, J=12.0, 5.1 Hz, 1H, H-6′b), 4.12 (d, J=7.6 Hz, 1H, H-6a), 4.03 (t, J=9.9 Hz, 1H, H-3′), 3.93-3.83 (m, 3H, H-5′, H-6b, H-3), 3.74 (dd, J=7.6, 4.8 Hz, 1H, H-5), 3.68 (t, J=9.9 Hz, 1H, H-4′), 3.50 (dd, J=8.6, 1.2 Hz, 1H, H-2), 3.42 (dd, J=9.9, 3.8 Hz, 1H, H-2′); ¹³C NMR (125 MHz, CDCl₃) δ 166.1 (C), 138.4 (C), 137.7 (C), 137.4 (C), 134.4 (C), 133.2 (CH), 133.13 (C), 133.09 (C), 129.5 (CH), 128.6 (CH), 128.5 (CH), 128.44 (CH), 128.36 (CH), 128.1 (CH), 128.0 (CH), 127.92 (CH), 127.88 (CH), 127.8 (CH), 127.7 (CH), 127.6 (CH), 127.1 (CH), 126.22 (CH), 126.15 (CH), 125.8 (CH), 99.51 (CH), 99.46 (CH), 82.8 (CH), 81.4 (CH), 79.9 (CH), 78.7 (CH), 78.1 (CH), 75.6 (CH₂), 75.5 (CH₂), 75.4 (CH₂), 74.2 (CH), 72.9 (CH₂), 70.0 (CH), 65.6 (CH₂), 63.5 (CH), 63.2 (CH₂). 45: ¹H NMR (500 MHz, CDCl₃) δ 7.96 (dd, J=8.4, 1.2 Hz, 2H, BzH), 7.66 (dd, J=6.0, 3.4 Hz, 1H, ArH), 7.60 (dd, J=6.0, 3.4 Hz, 1H, ArH), 7.54-7.50 (m, 2H, ArH), 7.42-7.23 (m, 20H, ArH), 7.09 (dd, J=8.4, 1.5 Hz, 1H, ArH), 5.77 (dd, J=10.7, 9.1 Hz, 1H, H-3′), 5.46 (d, J=3.8 Hz, 1H, H-1′), 5.33 (d, J=1.4 Hz, 1H, H-1), 4.93, 4.83 (ABq, J=10.7 Hz, 2H, CH₂Ar), 4.68 (s, 2H, CH₂Ar), 4.63, 4.51 (ABq, J=12.1 Hz, 2H, CH₂Ar), 4.62, 4.59 (ABq, J=11.0 Hz, 2H, CH₂Ar), 4.48 (t, J=4.7 Hz, 1H, H-5), 4.15 (d, J=7.9 Hz, 1H, H-6a), 3.95 (dd, J=7.5, 4.7 Hz, 1H, H-4), 3.92 (t, J=7.5 Hz, 1H, H-3), 3.92 (t, J=9.1 Hz, 1H, H-4′), 3.83 (ddd, J=9.1, 3.5, 1.5 Hz, 1H, H-5′), 3.80 (dd, J=10.6, 3.5 Hz, 1H, H-6′a), 3.74 (dd, J=7.9, 4.7 Hz, 1H, H-6b), 3.67 (dd, J=10.6, 1.5 Hz, 1H, H-6′b), 3.52 (dd, J=7.5, 1.4 Hz, 1H, H-2), 3.32 (dd, J=10.7, 3.8 Hz, 1H, H-2′); ¹³C NMR (125 MHz, CDCl₃) δ 165.4 (C), 138.3 (C), 137.7 (C), 137.5 (C), 134.5 (C), 133.3 (CH), 133.0 (C), 132.9 (C), 128.5 (CH), 128.4 (CH), 128.3 (CH), 128.2 (CH), 128.04 (CH), 127.99 (CH), 127.96 (C), 127.95 (CH), 127.87 (CH), 127.8 (CH), 127.59 (CH), 127.58 (CH), 127.0 (CH), 126.0 (CH), 125.93 (CH), 125.86 (CH), 99.5 (CH), 99.4 (CH), 83.2 (CH), 81.5 (CH), 77.4 (CH), 75.9 (CH), 75.3 (CH₂), 75.1 (CH₂), 74.2 (CH), 73.7 (CH₂), 72.8 (CH₂), 72.3 (CH), 71.1 (CH), 67.9 (CH₂), 65.8 (CH₂), 61.3 (CH); HRMS (FAB, MNa⁺) calcd for C₅₁H₄₉N₃O₁₀Na 886.3316, found 886.3329. 46: ¹H NMR (500 MHz, CDCl₃) δ 7.99-7.81 (m, 1H, ArH), 7.76-7.72 (m, 2H, ArH), 7.55 (s, 1H, ArH), 7.48-7.45 (m, 2H, ArH), 7.37-7.22 (m, 21H, ArH), 5.30 (d, J=1.6 Hz, 1H, H-1), 5.26 (d, J=3.8 Hz, 1H, H-1′), 4.92 (d, J=11.0 Hz, 1H, CH₂Ar), 4.92, 4.86 (ABq, J=10.7 Hz, 2H, CH₂Ar), 4.90 (s, 2H, CH₂Ar), 4.69, 4.67 (ABq, J=11.9 Hz, 2H, CH₂Ar), 4.64 (d, J=11.0 Hz, 1H, CH₂Ar), 4.55, 4.45 (ABq, J=12.1 Hz, 2H, CH₂Ar), 4.55 (t, J=4.0 Hz, 1H, H-5), 4.08 (d, J=7.9 Hz, 1H, H-6a), 3.94 (dd, J=10.3, 7.6 Hz, 1H, H-3′), 3.87 (dd, J=8.2, 4.0 Hz, 1H, H-4), 3.82 (dd, J=8.2, 7.7 Hz, 1H, H-3), 3.71-3.60 (m, 5H, H-4′, H-5′, H-6′a, H-6′b, H-6b), 3.50 (dd, J=7.7, 1.6 Hz, 1H, H-2), 3.39 (dd, J=10.3, 3.8 Hz, 1H, H-2′); ¹³C NMR (125 MHz, CDCl₃) δ 138.5 (C), 137.8 (C), 137.7 (C), 137.6 (C), 134.9 (C), 133.2 (C), 133.0 (C), 128.49 (CH), 128.45 (CH), 128.4 (CH), 128.3 (CH), 128.0 (CH), 127.87 (CH), 127.85 (CH), 127.7 (CH), 127.6 (CH), 126.8 (CH), 126.2 (CH), 126.1 (CH), 125.8 (CH), 99.7 (CH), 99.6 (CH), 82.8 (CH), 81.4 (CH), 79.9 (CH), 79.1 (CH), 78.2 (CH), 75.43 (CH₂×2), 75.36 (CH₂), 74.2 (CH), 73.6 (CH₂), 73.0 (CH₂), 71.5 (CH), 68.2 (CH₂), 65.7 (CH₂), 63.5 (CH); HRMS (FAB, MNa⁺) calcd for C₅₁H₅₁N₃O₉Na 872.3523, found 872.3528.

Synthesis of Compounds 47-62: To a solution of the disaccharide (31-46, 1.0 equiv) in wet chloroform (10 μL per 1 mg of the disaccharide) was consecutively added pyridine (10 μL per 1 mg of the disaccharide) and thioacetic acid (10 μL per 1 mg of the disaccharide) at room temperature under nitrogen. After stirring for 8 h, the mixture was diluted by ethyl acetate, and the mixture was sequentially washed with 1 N HCl (aq), saturated NaHCO₃ (aq), water, and brine. The organic layer was dried over anhydrous MgSO₄, filtered, and concentrated in vacuo to get a residue, which was purified by flash column chromatography on silica gel to afford the corresponding N-acetylated derivatives. The yields of compounds 47-62 are summarized in Scheme 4. 47: ¹H NMR (500 MHz, CDCl₃) δ 8.09 (dd, J=8.4, 1.2 Hz, 2H, ArH), 8.03 (dd, J=8.3, 1.1 Hz, 2H, ArH), 7.93 (dd, J=8.3, 1.2 Hz, 2H, ArH), 7.76 (dd, J=7.9, 1.5 Hz, 2H, ArH), 7.68-7.63 (m, 2H, ArH), 7.62-7.50 (m, 6H, ArH), 7.49-7.26 (m, 16H, ArH), 7.24-7.15 (m, 4H, ArH), 7.04-6.93 (m, 5H, ArH), 6.63 (d, J=10.0 Hz, 1H, NH), 5.74 (dd, J=10.8, 9.3 Hz, 1H, H-3′), 5.43 (dd, J=10.1, 9.4 Hz, 1H, H-2), 5.29 (d, J=3.2 Hz, 1H, H-1′), 4.80 (s, 2H, CH₂Ar), 4.74 (d, J=10.1 Hz, 1H, H-1), 4.66 (s, 2H, CH₂Ar), 4.55 (ddd, J=10.8, 10.0, 3.2 Hz, 1H, H-2′), 4.49 (dd, J=12.0, 3.9 Hz, 1H, H-6′a), 4.35 (dd, J=12.0, 1.8 Hz, 1H, H-6′b), 4.27-4.20 (m, 2H, H-6a, H-5′), 4.09 (t, J=9.4 Hz, 1H, H-4), 3.99-3.89 (m, 3H, H-3, H-6b, H-4′), 3.40-3.35 (m, 1H, H-5), 2.27 (s, 3H, CH₃), 1.00 (s, 9H, t-Bu), 0.91 (s, 3H, CH₃); ¹³C NMR (125 MHz, CDCl₃) δ 170.7 (C), 166.5 (C), 166.1 (C), 165.4 (C), 138.0 (C), 136.3 (C), 135.9 (CH), 135.5 (CH), 134.2 (C), 133.5 (CH), 133.3 (CH), 133.1 (CH), 133.0 (C), 132.8 (CH), 132.6 (C) 129.82 (CH), 129.79 (CH), 129.6 (CH), 129.1 (CH), 128.6 (CH), 128.5 (CH), 128.4 (CH), 128.3 (CH), 128.2 (CH), 127.8 (CH), 127.63 (CH), 127.59 (CH), 127.5 (CH), 127.4 (CH), 126.1 (CH), 125.94 (CH), 125.91 (CH), 100.1 (CH), 87.4 (CH), 83.3 (CH), 80.3 (CH), 77.9 (CH), 76.1 (CH₂), 75.7 (CH), 74.9 (CH₂), 73.8 (CH), 73.2 (CH), 70.8 (CH), 63.3 (CH₂), 62.4 (CH₂), 52.3 (CH), 26.8 (t-Bu), 21.4 (CH₃), 21.1 (CH₃), 19.3 (C); HRMS (FAB, MH⁺) calcd for C₇₆H₇₆NO₁₃SSi 1270.4807, found 1270.4838. 48: ¹H NMR (500 MHz, CDCl₃) δ 8.07 (dd, J=8.4, 1.3 Hz, 2H, ArH), 7.94 (dd, J=8.4, 1.3 Hz, 2H, ArH), 7.80-7.72 (m, 4H, ArH), 7.66-7.64 (m, 1H, ArH), 7.60-7.48 (m, 5H, ArH), 7.47-7.22 (m, 21H, ArH), 7.13 (dd, J=8.4, 1.6 Hz, 1H, ArH), 7.03-6.93 (m, 5H, ArH), 6.48 (d, J=10.0 Hz, 1H, NH), 5.62 (dd, J=11.0, 9.1 Hz, 1H, H-3′), 5.40 (t, J=9.5 Hz, 1H, H-2), 5.25 (d, J=3.3 Hz, 1H, H-1′), 4.75 (s, 2H, CH₂Ar), 4.74 (d, J=9.5 Hz, 1H, H-1), 4.61 (s, 2H, CH₂Ar), 4.55 (d, J=11.9 Hz, 1H, CH₂Ar), 4.50 (ddd, J=1.0, 10.0, 3.3 Hz, 1H, H-2′), 4.36 (d, J=11.9 Hz, 1H, CH₂Ar), 4.27 (dd, J=11.5, 3.2 Hz, 1H, H-6a), 4.07-3.93 (m, 4H, H-4, H-6b, H-4′, H-5′), 3.93 (t, J=9.5 Hz, 1H, H-3), 3.69 (dd, J=10.9, 3.6 Hz, 1H, H-6′a), 3.49 (dd, J=10.9, 1.4 Hz, 1H, H-6′b), 3.39 (ddd, J=9.5, 3.2, 1.6 Hz, 1H, H-5), 2.26 (s, 3H, CH₃), 1.03 (s, 9H, t-Bu), 0.94 (s, 3H, CH₃); ¹³C NMR (125 MHz, CDCl₃) δ170.5 (C), 166.6 (C), 165.4 (C), 138.1 (C), 137.9 (C), 136.3 (C), 136.0 (CH), 135.6 (CH), 135.0 (C), 133.4 (CH), 133.1 (CH), 132.90 (C), 132.86 (C), 132.5 (CH), 129.8 (CH), 129.7 (CH), 129.6 (CH), 129.0 (CH), 128.6 (CH), 128.4 (CH), 128.3 (CH), 128.2 (CH), 128.1 (CH), 127.9 (CH), 127.72 (CH), 127.67 (CH), 127.6 (CH), 127.1 (CH), 126.0 (CH), 125.9 (CH), 125.8 (CH), 100.0 (CH), 87.5 (CH), 83.5 (CH), 80.4 (CH), 77.4 (CH), 76.0 (CH), 75.8 (CH₂), 74.8 (CH₂), 73.8 (CH), 73.5 (CH₂), 73.2 (CH), 72.6 (CH), 68.8 (CH₂), 63.6 (CH₂), 52.3 (CH), 26.9 (t-Bu), 21.6 (CH₃), 21.1 (CH₃), 19.4 (C); HRMS (FAB, MH⁺) calcd for C₇₆H₇₈NO₁₂SSi 1256.5014, found 1256.5020. 49: ¹H NMR (400 MHz, CDCl₃) δ 8.04 (dd, J=8.4, 1.3 Hz, 2H, ArH), 7.87 (dd, J=8.4, 1.3 Hz, 2H, ArH), 7.77-7.61 (m, 8H, ArH), 7.56 (tt, J=7.4, 1.3 Hz, 1H, ArH), 7.49 (tt, J=7.4, 1.3 Hz, 1H, ArH), 7.45-7.36 (m, 6H, ArH), 7.34-7.14 (m, 16H, ArH), 7.09-7.04 (m, 3H, ArH), 6.95 (d, J=7.9 Hz, 2H, ArH), 6.57 (d, J=9.9 Hz, 1H, NH), 5.39 (dd, J=10.1, 9.3 Hz, 1H, H-2), 5.23 (d, J=3.2 Hz, 1H, H-1′), 4.96, 4.73 (ABq, J=10.9 Hz, 2H, CH₂Ar), 4.78-4.73 (m, 2H, CH₂Ar), 4.72 (d, J=10.1 Hz, 1H, H-1), 4.60 (d, J=10.4 Hz, 1H, CH₂Ar), 4.56 (d, J=11.0 Hz, 1H, CH₂Ar), 4.46-4.36 (m, 3H, H-2′, H-6′a, H-6′b), 4.20 (dd, J=11.7, 2.9 Hz, 1H, H-6a), 4.11-4.08 (m, 1H, H-5′), 4.07 (t, J=9.3 Hz, 1H, H-4), 3.91 (dd, J=11.7, 1.2 Hz, 1H, H-6b), 3.89 (t, J=9.3 Hz, 1H, H-3), 3.81-3.74 (m, 2H, H-3′, H-4′), 3.33 (ddd, J=9.3, 2.9, 1.2 Hz, 1H, H-5), 2.27 (s, 3H, CH₃), 1.35 (s, 3H, CH₃), 1.00 (s, 9H, t-Bu); ¹³C NMR (125 MHz, CDCl₃) δ 170.3 (C), 166.1 (C), 165.3 (C), 138.2 (C), 138.0 (C), 136.2 (C), 135.9 (CH), 135.5 (CH), 134.9 (C), 133.4 (CH), 133.1 (C), 133.0 (CH), 132.9 (C), 132.8 (CH), 132.7 (C), 130.9 (CH), 129.8 (CH), 129.6 (CH), 129.52 (CH), 129.47 (C), 128.8 (CH), 128.6 (CH), 128.5 (CH), 128.42 (CH), 128.38 (CH), 128.34 (CH), 128.29 (CH), 127.9 (CH), 127.6 (CH), 127.5 (CH), 127.2 (CH), 126.1 (CH), 126.04 (CH), 125.98 (CH), 99.9 (CH), 87.3 (CH), 83.6 (CH), 81.7 (CH), 80.4 (CH), 77.4 (CH), 77.1 (CH), 75.9 (CH₂), 75.2 (CH₂), 74.8 (CH₂), 73.0 (CH), 70.7 (CH), 63.4 (CH₂), 62.4 (CH₂), 53.3 (CH), 26.8 (t-Bu), 22.4 (CH₃), 21.1 (CH₃), 19.3 (C); HRMS (FAB, MH⁺) calcd for C₇₆H₇₈NO₁₂SSi 1256.5014, found 1256.5040. 50: ¹H NMR (500 MHz, CDCl₃) δ 8.03 (dd, J=8.3, 1.2 Hz, 2H, ArH), 7.83-7.72 (m, 6H, ArH), 7.61-7.53 (m, 2H, ArH), 7.48-7.40 (m, 4H, ArH), 7.33-7.19 (m, 21H, ArH), 7.16-7.13 (m, 2H, ArH), 7.09-7.03 (m, 2H, ArH), 6.94 (d, J=8.1 Hz, 2H, ArH), 6.41 (d, J=10.1 Hz, 1H, NH), 5.38 (dd, J=9.9, 9.2 Hz, 1H, H-2), 5.20 (d, J=3.4 Hz, 1H, H-1′), 4.91 (d, J=10.9 Hz, 1H, CH₂Ar), 4.76-4.71 (m, 3H, H-1, CH₂Ar), 4.58-4.53 (m, 3H, CH₂Ar), 4.49 (d, J=11.9 Hz, 1H, CH₂Ar), 4.39 (td, J=10.1, 3.4, 1H, H-2′), 4.34 (d, J=11.9 Hz, 1H, CH₂Ar), 4.24 (dd, J=11.6, 3.5 Hz, 1H, H-6a), 4.03 (t, J=9.2 Hz, 1H, H-4), 4.00 (dd, J=11.6, 1.6 Hz, 1H, H-6b), 3.91 (ddd, J=9.6, 4.1, 1.2 Hz, 1H, H-5′), 3.86 (t, J=9.2 Hz, 1H, H-3), 3.79 (dd, J=9.6, 9.0 Hz, 1H, H-4′), 3.71 (dd, J=10.1, 9.0 Hz, 1H, H-3′), 3.64 (dd, J=10.7, 4.1 Hz, 1H, H-6′a), 3.52 (dd, J=10.7, 1.2 Hz, 1H, H-6′b), 3.40-3.36 (ddd, J=9.2, 3.5, 1.6 Hz, 1H, H-5), 2.26 (s, 3H, CH₃), 1.35 (s, 3H, CH₃), 1.04 (s, 9H, t-Bu); ¹³C NMR (125 MHz, CDCl₃) δ 170.1 (C), 165.2 (C), 138.4 (C), 138.1 (C), 137.8 (C), 136.2 (C), 135.9 (CH), 135.6 (CH), 135.5 (C), 133.4 (CH), 133.3 (C), 133.2 (C), 133.0 (C), 132.9 (C), 132.5 (CH), 129.8 (CH), 129.6 (CH), 128.5 (CH), 128.3 (CH), 128.1 (CH), 127.9 (CH), 127.7 (CH), 127.6 (CH), 127.53 (CH), 127.49 (CH), 127.4 (CH), 126.8 (CH), 126.0 (CH), 125.9 (CH), 99.7 (CH), 87.3 (CH), 83.6 (CH), 81.3 (CH), 80.4 (CH), 77.6 (CH), 76.9 (CH), 75.6 (CH₂), 75.0 (CH₂), 74.3 (CH₂), 73.4 (CH₂), 72.9 (CH), 72.6 (CH), 69.1 (CH₂), 62.6 (CH₂), 53.0 (CH), 26.8 (t-Bu), 22.4 (CH₃), 21.1 (CH₃), 19.3 (C); HRMS (FAB, MH⁺) calcd for C₇₆H₈₀NO₁₁SSi 1242.5221, found 1242.5225. 51: ¹H NMR (500 MHz, CDCl₃) δ 8.02 (d, J=7.4 Hz, 2H, ArH), 7.91 (d, J=7.3 Hz, 2H, ArH), 7.73 (dd, J=7.8, 1.2 Hz, 2H, ArH), 7.66-7.64 (m, 2H, ArH), 7.61-7.50 (m, 5H, ArH), 7.46 (s, 1H, ArH), 7.42-7.25 (m, 18H, ArH), 7.22-7.17 (m, 4H, ArH), 7.13-7.11 (m, 3H, ArH), 6.99 (d, J=8.0 Hz, 2H, ArH), 6.54 (d, J=10.0 Hz, 1H, NH), 5.69 (dd, J=10.8, 9.3 Hz, 1H, H-3′), 5.30 (d, J=3.3 Hz, 1H, H-1′), 5.16 (d, J=10.2 Hz, 1H, CH₂Ar), 5.09 (d, J=10.2 Hz, 1H, CH₂Ar), 4.79 (d, J=10.2 Hz, 1H, CH₂Ar), 4.78 (d, J=10.2 Hz, 1H, CH₂Ar), 4.64 (s, 2H, CH₂Ar), 4.62 (d, J=9.8 Hz, 1H, H-1), 4.58-4.50 (m, 1H, H-2′), 4.45 (dd, J=12.0, 4.0 Hz, 1H, H-6′a), 4.32 (dd, J=12.0, 1.8 Hz, 1H, H-6′b), 4.20 (dd, J=11.9, 2.9 Hz, 1H, H-6a), 4.19-4.15 (m, 1H, H-5′), 3.95 (t, J=9.1 Hz, 1H, H-4), 3.93-3.89 (m, 2H, H-6b, H-4′), 3.73 (t, J=9.1 Hz, 1H, H-3), 3.62 (dd, J=9.8, 9.1 Hz, 1H, H-2), 3.28-3.27 (m, 1H, H-5), 2.29 (s, 3H, CH₃), 1.01 (s, 3H, CH₃), 0.99 (s, 9H, t-Bu); ¹³C NMR (100 MHz, CDCl₃) δ 170.5 (C), 166.5 (C), 166.1 (C), 137.7 (C), 137.6 (C), 137.1 (C), 135.9 (CH), 135.5 (CH), 134.3 (C), 133.2 (CH), 133.1 (CH), 133.03 (C), 132.98 (C), 132.96 (C), 132.8 (C), 132.1 (CH), 130.5 (C), 129.8 (CH), 129.7 (CH), 129.6 (CH), 128.9 (CH), 128.51 (CH), 128.47 (CH), 128.4 (CH), 128.3 (CH), 128.2 (CH), 128.1 (CH), 128.0 (CH), 127.8 (CH), 127.63 (CH), 127.61 (CH), 127.56 (CH), 127.4 (CH), 126.1 (CH), 125.94 (CH), 125.90 (CH), 99.6 (CH), 88.5 (CH), 84.9 (CH), 81.6 (CH), 79.9 (CH), 77.5 (CH), 76.3 (CH₂), 75.7 (CH), 75.1 (CH₂), 74.9 (CH₂), 73.9 (CH), 70.6 (CH), 63.3 (CH₂), 62.7 (CH₂), 52.3 (CH), 26.8 (t-Bu), 21.6 (CH₃), 21.1 (CH₃), 19.3 (C); HRMS (FAB, MH⁺) calcd for C₇₆H₇₈NO₁₂SSi 1256.5014, found 1256.5005. 52: ¹H NMR (500 MHz, CDCl₃) δ 7.86 (d, J=8.0 Hz, 2H, ArH), 7.74-7.68 (m, 5H, ArH), 7.65-7.62 (m, 3H, ArH), 7.48 (t, J=7.4 Hz, 1H, ArH), 7.43-7.39 (m, 4H, ArH), 7.37-7.16 (m, 24H, ArH), 6.99 (d, J=7.8 Hz, 2H, ArH), 6.53 (d, J=9.9 Hz, 1H, NH), 5.22 (d, J=3.2 Hz, 1H, H-1′), 5.13 (d, J=10.9 Hz, 1H, CH₂Ar), 5.05 (d, J=10.1 Hz, 1H, CH₂Ar), 4.94 (d, J=10.9 Hz, 1H, CH₂Ar), 4.74-4.67 (m, 4H, CH₂Ar), 4.61 (t, J=9.5 Hz, 1H, H-1), 4.51 (d, J=1.0 Hz, 1H, CH₂Ar), 4.44-4.33 (m, 3H, H-2′, H-6′a, H-6′b), 4.16 (dd, J=11.5, 2.8 Hz, 1H, H-6a), 4.05-4.03 (m, 1H, H-5′), 3.95 (t, J=9.1 Hz, 1H, H-4), 3.88 (d, J=11.5 Hz, 1H, H-6b), 3.77 (t, J=8.9 Hz, 1H, H-4′), 3.74 (t, J=8.9 Hz, 1H, H-3′), 3.68 (t, J=9.1 Hz, 1H, H-3), 3.61 (dd, J=9.5, 9.1 Hz, 1H, H-2), 3.25-3.23 (m, 1H, H-5), 2.29 (s, 3H, CH₃), 1.44 (s, 3H, CH₃), 1.00 (s, 9H, i-Bu); ¹³C NMR (125 MHz, CDCl₃) δ 170.1 (C), 166.1 (C), 138.3 (C), 137.6 (C), 137.1 (C), 135.8 (CH), 135.5 (CH), 135.0 (C), 133.2 (C), 133.04 (C), 133.01 (CH), 132.94 (C), 132.90 (C), 132.1 (CH), 130.4 (C), 129.7 (CH), 129.64 (CH), 129.58 (CH), 129.5 (CH), 128.8 (CH), 128.7 (CH), 128.5 (CH), 128.4 (CH), 128.3 (CH), 128.2 (CH), 128.03 (CH), 127.96 (CH), 127.9 (CH), 127.6 (CH), 127.5 (CH), 127.2 (CH), 126.1 (CH), 126.0 (CH), 99.5 (CH), 88.4 (CH), 85.0 (CH), 81.7 (CH), 81.4 (CH), 80.0 (CH), 77.2 (CH), 77.0 (CH), 75.2 (CH₂), 74.8 (CH₂×2), 74.5 (CH₂), 70.6 (CH), 63.4 (CH₂), 62.6 (CH₂), 53.3 (CH), 26.8 (t-Bu), 22.6 (CH₃), 21.1 (CH₃), 19.3 (C); HRMS (FAB, MH⁺) calcd for C₇₆H₈₀NO₁₁SSi 1242.5221, found 1242.5220. 53: ¹H NMR (400 MHz, CDCl₃) δ 7.94 (dd, J=8.2, 1.2 Hz, 2H, ArH), 7.77-7.71 (m, 4H, ArH), 7.67-7.63 (m, 1H, ArH), 7.59-7.56 (m, 1H, ArH), 7.52-7.48 (m, 2H, ArH), 7.44-7.25 (m, 25H, ArH), 7.16-7.11 (m, 4H, ArH), 6.99 (d, J=8.2 Hz, 2H, ArH), 6.37 (d, J=9.9 Hz, 1H, NH), 5.60-5.54 (m, 1H, H-3′), 5.29 (d, J=3.4 Hz, 1H, H-1′), 5.11 (d, J=10.2 Hz, 1H, CH₂Ar), 5.07 (d, J=10.2 Hz, 1H, CH₂Ar), 4.77 (d, J=10.2 Hz, 1H, CH₂Ar), 4.73 (d, J=10.2 Hz, 1H, CH₂Ar), 4.64 (d, J=9.6 Hz, 1H, H-1), 4.59 (s, 2H, CH₂Ar), 4.54 (d, J=11.9 Hz, 1H, CH₂Ar), 4.50 (td, J=9.9, 3.4 Hz, 1H, H-2′), 4.33 (d, J=11.9 Hz, 1H, CH₂Ar), 4.21 (dd, J=11.6, 3.5 Hz, 1H, H-6a), 3.98 (dd, J=11.6, 1.8 Hz, 1H, H-6b), 3.96-3.90 (m, 3H, H-4, H-4′, H-5′), 3.69 (t, J=8.7 Hz, 1H, H-3), 3.64 (dd, J=10.6, 2.3 Hz, 1H, H-6′a), 3.60 (dd, J=9.6, 8.7 Hz, 1H, H-2), 3.45 (d, J=10.6 Hz, 1H, H-6′b), 3.31 (ddd, J=9.1, 3.5, 1.8 Hz, 1H, H-5), 2.29 (s, 3H, CH₃), 1.06 (s, 3H, CH₃), 1.04 (s, 9H, t-Bu); ¹³C NMR (125 MHz, CDCl₃) δ 170.3 (C), 166.5 (C), 138.1 (C), 137.7 (C), 137.5 (C), 137.2 (C), 135.9 (CH), 135.6 (CH), 135.0 (C), 133.5 (C), 133.0 (CH), 132.9 (C), 131.9 (CH), 130.7 (C), 129.7 (CH), 129.6 (CH), 128.8 (CH), 128.5 (CH), 128.4 (CH), 128.03 (CH), 127.98 (CH), 127.9 (CH), 127.7 (CH), 127.61 (CH), 127.57 (CH), 127.0 (CH), 126.2 (CH), 125.9 (CH), 125.8 (CH), 99.5 (CH), 88.5 (CH), 85.1 (CH), 81.6 (CH), 79.9 (CH), 76.8 (CH), 76.00 (CH₂), 75.97 (CH), 75.1 (CH₂), 74.8 (CH₂), 73.9 (CH), 73.5 (CH₂), 72.3 (CH), 68.7 (CH₂), 62.9 (CH₂), 52.3 (CH), 26.9 (t-Bu), 21.8 (CH₃), 21.1 (CH₃), 19.4 (C); HRMS (FAB, MH⁺) calcd for C₇₆H₈₀NO₁₁SSi 1242.5221, found 1242.5223. 54: ¹H NMR (500 MHz, CDCl₃) δ 7.84-7.72 (m, 7H, ArH), 7.59 (s, 1H, ArH), 7.49-7.41 (m, 4H, ArH), 7.37-7.19 (m, 27H, ArH), 7.00 (d, J=7.9 Hz, 2H, ArH), 6.36 (d, J=10.1 Hz, 1H, NH), 5.23 (d, J=3.5 Hz, 1H, H-1′), 5.08 (d, J=10.9 Hz, 1H, CH₂Ar), 5.04 (d, J=10.2 Hz, 1H, CH₂Ar), 4.91 (d, J=10.9 Hz, 1H, CH₂Ar), 4.74 (d, J=11.1 Hz, 1H, CH₂Ar), 4.73 (d, J=10.1 Hz, 1H, CH₂Ar), 4.68-4.62 (m, 3H, CH₂Ar, H-1), 4.52 (d, J=11.1 Hz, 1H, CH₂Ar), 4.50 (d, J=11.9 Hz, 1H, CH₂Ar), 4.41 (td, J=10.1, 3.5 Hz, 1H, H-2′), 4.33 (d, J=11.9 Hz, 1H, CH₂Ar), 4.20 (dd, J=11.6, 3.7 Hz, 1H, H-6a), 3.98 (dd, J=11.6, 1.6 Hz, 1H, H-6b), 3.94 (t, J=8.9 Hz, 1H, H-4), 3.90-3.85 (m, 1H, H-5), 3.79 (dd, J=9.9, 8.9 Hz, 1H, H-4′), 3.69 (dd, J=10.1, 8.9 Hz, 1H, H-3′), 3.68-3.58 (m, 3H, H-2, H-3, H-6′a), 3.49 (dd, J=10.9, 1.7 Hz, 1H, H-6′b), 3.39-3.28 (m, 1H, H-5′), 2.29 (s, 3H, CH₃), 1.45 (s, 3H, CH₃), 1.06 (s, 9H, t-Bu); ¹³C NMR (125 MHz, CDCl₃) δ 169.9 (C), 138.5 (C), 138.1 (C), 137.6 (C), 137.5 (C), 137.2 (C), 135.9 (CH), 135.6 (CH), 133.3 (C), 133.2 (C), 133.1 (C), 133.0 (C), 131.9 (CH), 130.6 (C), 129.7 (CH), 129.60 (CH), 129.57 (CH), 128.6 (CH), 128.5 (CH), 128.3 (CH), 128.14 (CH), 128.08 (CH), 127.99 (CH), 127.95 (CH), 127.89 (CH), 127.7 (CH), 127.64 (CH), 127.57 (CH), 127.5 (CH), 127.4 (CH), 126.8 (CH), 126.1 (CH), 126.0 (CH), 125.9 (CH), 99.3 (CH), 88.3 (CH), 85.1 (CH), 81.3 (CH×2), 80.1 (CH), 77.6 (CH), 76.3 (CH), 75.7 (CH₂), 75.1 (CH₂), 75.0 (CH₂), 74.4 (CH₂), 73.4 (CH₂), 72.4 (CH), 69.0 (CH₂), 62.9 (CH₂), 53.0 (CH), 26.9 (t-Bu), 22.6 (CH₃), 21.1 (CH₃), 19.4 (C); HRMS (FAB, MH⁺) calcd for C₇₆H₈₂NO₁₀SSi 1228.5429, found 1228.5413. 55: ¹H NMR (500 MHz, CDCl₃) δ 8.05 (dd, J=8.4, 1.2 Hz, 2H, BzH), 8.02 (dd, J=8.4, 1.2 Hz, 2H, BzH), 7.96 (dd, J=8.4, 1.2 Hz, 2H, BzH), 7.65-7.55 (m, 6H, ArH), 7.51 (s, 1H, ArH), 7.46-7.36 (m, 8H, ArH), 7.28-7.12 (m, 6H, ArH), 5.78 (d, J=9.7 Hz, 1H, NH), 5.69 (dd, J=11.0, 9.4 Hz, 1H, H-3′), 5.51 (d, J=1.7 Hz, 1H, H-1), 5.10 (dd, J=8.3, 1.7 Hz, 1H, H-2), 5.06 (d, J=3.6 Hz, 1H, H-1′), 4.76, 4.69 (ABq, J=10.8 Hz, 2H, CH₂Ar), 4.75, 4.73 (ABq, J=10.8 Hz, 2H, CH₂Ar), 4.71 (dd, J=5.2, 4.7 Hz, 1H, H-5), 4.60-4.54 (m, 2H, H-6′a, H-6′b), 4.50 (ddd, J=11.0, 9.7, 3.6 Hz, 1H, H-2′), 4.17 (d, J=7.8 Hz, 1H, H-6a), 4.12-4.09 (m, 1H, H-5′), 4.09 (t, J=8.3 Hz, 1H, H-3), 3.93 (t, J=9.4 Hz, 1H, H-4′), 3.92 (dd, J=8.3, 4.7 Hz, 1H, H-4), 3.75 (dd, J=7.8, 5.2 Hz, 1H, H-6b), 1.18 (s, 3H, CH₃); ¹³C NMR (125 MHz, CDCl₃) δ 170.3 (C), 166.7 (C), 166.1 (C), 165.6 (C), 137.4 (C), 134.1 (C), 133.53 (CH), 133.50 (CH), 133.3 (CH), 133.03 (C), 132.98 (C), 129.84 (CH), 129.76 (CH), 129.6 (CH), 129.4 (C), 129.3 (C), 129.2 (C), 128.7 (CH), 128.6 (CH), 128.54 (CH), 128.46 (CH), 128.13 (CH), 128.07 (CH), 127.8 (CH), 127.6 (CH), 127.4 (CH), 126.09 (CH), 126.06 (CH), 125.9 (CH), 99.9 (CH), 99.3 (CH), 80.2 (CH), 79.1 (CH), 76.9 (CH), 75.6 (CH), 75.2 (CH₂), 75.1 (CH₂), 73.8 (CH), 73.6 (CH), 70.8 (CH), 65.3 (CH₂), 63.1 (CH₂), 52.1 (CH), 22.1 (CH₃); HRMS (FAB, MH⁺) calcd for C₅₃H₅₀NO₁₃ 908.3282, found 908.3274. 56: ¹H NMR (400 MHz, CDCl₃) δ 8.05 (dd, J=8.4, 1.3 Hz, 2H, BzH), 7.96 (dd, J=8.4, 1.3 Hz, 2H, BzH), 7.71-7.67 (m, 1H, ArH), 7.62-7.51 (m, 4H, ArH), 7.46-7.02 (m, 18H, ArH), 5.72 (d, J=9.6 Hz, 1H, NH), 5.59 (dd, J=10.8, 8.8 Hz, 1H, H-3′), 5.52 (d, J=1.7 Hz, 1H, H-1), 5.10 (dd, J=8.3, 1.7 Hz, 1H, H-2), 5.09 (d, J=3.8 Hz, 1H, H-1′), 4.78 (t, J=4.5 Hz, 1H, H-5), 4.75, 4.69 (ABq, J=10.8 Hz, 2H, CH₂Ar), 4.68, 4.61 (ABq, J=11.1 Hz, 2H, CH₂Ar), 4.59, 4.53 (ABq, J=12.0 Hz, 2H, CH₂Ar), 4.47 (ddd, J=10.8, 9.6, 3.8 Hz, 1H, H-2′), 4.14 (d, J=7.8 Hz, 1H, H-6a), 4.07 (t, J=8.3 Hz, 1H, H-3), 3.94-3.88 (m, 3H, H-4, H-4′, H-5′), 3.78-3.67 (m, 2H, H-6′a, H-6′b), 3.75 (dd, J=7.8, 4.5 Hz, 1H, H-6b), 1.22 (s, 3H, CH₃); ¹³C NMR (100 MHz, CDCl₃) δ 170.2 (C), 166.8 (C), 165.6 (C), 137.7 (C), 137.5 (C), 134.6 (C), 133.5 (CH), 133.3 (CH), 133.0 (C), 132.9 (C), 129.8 (CH), 129.7 (CH), 129.33 (C), 129.26 (C), 128.6 (CH), 128.51 (CH), 128.48 (CH), 128.21 (CH), 128.1 (CH), 127.94 (CH), 127.87 (CH), 127.84 (CH), 127.79 (CH), 127.6 (CH), 127.0 (CH), 126.03 (CH), 125.96 (CH), 125.9 (CH), 100.0 (CH), 99.3 (CH), 80.3 (CH), 79.1 (CH), 76.9 (CH), 75.9 (CH), 75.2 (CH₂), 75.1 (CH₂), 73.8 (CH), 73.64 (CH), 73.61 (CH₂), 72.3 (CH), 68.4 (CH₂), 65.5 (CH₂), 52.2 (CH), 22.2 (CH₃); HRMS (FAB, MH⁺) calcd for C₅₃H₅₂NO₁₂ 894.3490, found 894.3507. 57: ¹H NMR (500 MHz, CDCl₃) δ 8.01 (dd, J=8.4, 1.4 Hz, 2H, BzH), 7.93 (dd, J=8.4, 1.4 Hz, 2H, BzH), 7.79-7.73 (m, 3H, ArH), 7.70 (s, 1H, ArH), 7.60-7.50 (m, 2H, ArH), 7.44-7.37 (m, 7H, ArH), 7.34-7.26 (m, 5H, ArH), 7.18-7.17 (m, 5H, ArH), 5.48 (d, J=1.6 Hz, 1H, H-1), 5.41 (d, J=9.3 Hz, 1H, NH), 5.08 (dd, J=8.3, 1.6 Hz, 1H, H-2), 5.02, 4.80 (ABq, J=10.9 Hz, 2H, CH₂Ar), 4.98 (d, J=3.6 Hz, 1H, H-1′), 4.86, 4.68 (ABq, J=11.3 Hz, 2H, CH₂Ar), 4.70 (dd, J=5.4, 3.9 Hz, 1H, H-5), 4.68, 4.57 (ABq, J=11.1 Hz, 2H, CH₂Ar), 4.57 (dd, J=11.9, 2.1 Hz, 1H, H-6′a), 4.46 (dd, J=11.9, 4.8 Hz, 1H, H-6′b), 4.35 (td, J=9.3, 3.6 Hz, 1H, H-2′), 4.08 (d, J=7.7 Hz, 1H, H-6a), 4.00 (t, J=8.3 Hz, 1H, H-3), 3.96 (ddd, J=9.5, 4.8, 2.1 Hz, 1H, H-5′), 3.89 (dd, J=8.3, 3.9 Hz, 1H, H-4), 3.78-3.74 (m, 2H, H-3′, H-4′), 3.70 (dd, J=7.7, 5.4 Hz, 1H, H-6b), 1.41 (s, 3H, CH₃); ¹³C NMR (125 MHz, CDCl₃) δ 170.1 (C), 166.1 (C), 165.5 (C), 138.0 (C), 137.4 (C), 134.6 (C), 133.5 (CH), 133.3 (CH), 133.2 (C), 133.1 (C), 129.8 (CH), 129.5 (CH), 129.4 (C), 129.2 (C), 128.7 (CH), 128.6 (CH), 128.5 (CH), 128.2 (CH), 128.1 (CH), 128.0 (CH), 127.9 (CH), 127.71 (CH), 127.66 (CH), 127.3 (CH), 126.2 (CH), 126.1 (CH), 126.0 (CH), 100.2 (CH), 99.3 (CH), 80.5 (CH), 80.1 (CH), 79.0 (CH), 77.7 (CH), 77.3 (CH), 75.4 (CH₂), 75.3 (CH₂), 74.7 (CH₂), 73.7 (CH), 70.9 (CH), 65.2 (CH₂), 63.2 (CH₂), 52.7 (CH), 22.7 (CH₃); HRMS (FAB, MH⁺) calcd for C₅₃H₅₂NO₁₂ 894.3490, found 894.3500. 58: ¹H NMR (500 MHz, CDCl₃) δ 8.01 (dd, J=8.4, 1.3 Hz, 2H, BzH), 7.83-7.76 (m, 3H, ArH), 7.61 (s, 1H, ArH), 7.58-7.54 (m, 1H, ArH), 7.50-7.41 (m, 4H, ArH), 7.34-7.26 (m, 11H, ArH), 7.19-7.14 (m, 5H, ArH), 5.49 (d, J=1.6 Hz, 1H, H-1), 5.31 (d, J=9.4 Hz, 1H, NH), 5.07 (dd, J=8.3, 1.6 Hz, 1H, H-2), 4.98 (d, J=3.6 Hz, 1H, H-1′), 4.94, 4.66 (ABq, J=10.8 Hz, 2H, CH₂Ar), 4.85, 4.66 (ABq, J=11.4 Hz, 2H, CH₂Ar), 4.80 (dd, J=5.2, 4.3 Hz, 1H, H-5), 4.67, 4.54 (ABq, J=11.2 Hz, 2H, CH₂Ar), 4.54, 4.47 (ABq, J=12.1 Hz, 2H, CH₂Ar), 4.35 (ddd, J=10.3, 9.4, 3.6 Hz, 1H, H-2′), 4.07 (d, J=7.8 Hz, 1H, H-6a), 3.98 (t, J=8.3 Hz, 1H, H-3), 3.87 (dd, J=8.3, 4.3 Hz, 1H, H-4), 3.78-3.76 (m, 1H, H-5′), 3.71 (dd, J=7.8, 5.2 Hz, 1H, H-6b), 3.69-3.64 (m, 4H, H-3′, H-4′, H-6′a, H-6′b), 1.37 (s, 3H, CH₃); ¹³C NMR (125 MHz, CDCl₃) δ 170.0 (C), 165.6 (C), 138.2 (C), 137.7 (C), 137.5 (C), 135.1 (C), 133.5 (CH), 133.2 (C), 133.1 (C), 129.8 (CH), 129.3 (C), 128.6 (CH), 128.53 (CH), 128.47 (CH), 128.45 (CH), 128.3 (CH), 128.10 (CH), 128.05 (CH), 127.92 (CH), 127.89 (CH), 127.8 (CH), 127.74 (CH), 127.68 (CH), 127.6 (CH), 127.0 (CH), 126.2 (CH), 126.1 (CH), 126.0 (CH), 100.3 (CH), 99.3 (CH), 80.4 (CH), 80.3 (CH), 78.9 (CH), 78.2 (CH), 76.7 (CH), 75.3 (CH₂), 74.9 (CH₂), 74.7 (CH₂), 73.9 (CH), 73.5 (CH₂), 72.3 (CH), 68.6 (CH₂), 65.5 (CH₂), 52.6 (CH), 22.7 (CH₃); HRMS (FAB, MH⁺) calcd for C₅₃H₅₄NO₁₁ 880.3697, found 880.3706. 59: ¹H NMR (400 MHz, CDCl₃) δ 8.00 (dd, J=8.4, 1.3 Hz, 2H, BzH), 7.94 (dd, J=8.4, 1.3 Hz, 2H, BzH), 7.64-7.51 (m, 5H, ArH), 7.49 (s, 1H, ArH), 7.43-7.21 (m, 17H, ArH), 5.74 (d, J=9.6 Hz, 1H, NH), 5.64 (dd, J=11.0, 9.6 Hz, 1H, H-3′), 5.32 (d, J=1.4 Hz, 1H, H-1), 5.03 (d, J=3.6 Hz, 1H, H-1′), 4.96, 4.68 (ABq, J=10.9 Hz, 2H, CH₂Ar), 4.73, 4.71 (ABq, J=11.1 Hz, 2H, CH₂Ar), 4.67, 4.66 (ABq, J=11.9 Hz, 2H, CH₂Ar), 4.60 (dd, J=5.1, 4.1 Hz, 1H, H-5), 4.57-4.52 (m, 2H, H-6′a, H-6′b), 4.50 (ddd, J=11.0, 9.6, 3.6 Hz, 1H, H-2′), 4.08 (d, J=7.8 Hz, 1H, H-6a), 4.05 (dt, J=9.6, 3.2 Hz, 1H, H-5′), 3.90 (t, J=9.6 Hz, 1H, H-4′), 3.81 (dd, J=8.5, 7.6 Hz, 1H, H-3), 3.76 (dd, J=8.5, 4.1 Hz, 1H, H-4), 3.71 (dd, J=7.8, 5.1 Hz, 1H, H-6b), 3.53 (dd, J=7.6, 1.4 Hz, 1H, H-2), 1.19 (s, 3H, CH₃); ¹³C NMR (125 MHz, CDCl₃) δ 170.3 (C), 166.7 (C), 166.1 (C), 138.0 (C), 137.5 (C), 134.1 (C), 133.4 (CH), 133.3 (CH), 133.02 (C), 132.97 (C), 129.7 (CH), 129.6 (CH), 129.5 (C), 129.3 (C), 128.64 (CH), 128.59 (CH), 128.57 (CH), 128.50 (CH), 128.45 (CH), 128.1 (CH), 128.0 (CH), 127.9 (CH), 127.8 (CH), 127.6 (CH), 127.4 (CH), 126.1 (CH), 126.0 (CH), 125.9 (CH), 99.8 (CH), 99.3 (CH), 83.0 (CH), 80.7 (CH), 80.1 (CH), 75.6 (CH₂), 75.5 (CH), 75.2 (CH₂), 73.8 (CH), 73.6 (CH), 72.7 (CH₂), 70.6 (CH), 65.3 (CH₂), 63.1 (CH₂), 52.1 (CH), 22.2 (CH₃); HRMS (FAB, MH⁺) calcd for C₅₃H₅₂NO₁₂ 894.3490, found 894.3500. 60: ¹H NMR (500 MHz, CDCl₃) δ 7.91 (dd, J=8.2, 1.2 Hz, 2H, BzH), 7.78-7.72 (m, 3H, ArH), 7.68 (s, 1H, ArH), 7.54-7.50 (m, 1H, ArH), 7.45-7.35 (m, 5H, ArH), 7.34-7.22 (m, 15H, ArH), 5.36 (d, J=9.7 Hz, 1H, NH), 5.31 (d, J=1.5 Hz, 1H, H-1), 5.00, 4.78 (ABq, J=10.9 Hz, 2H, CH₂Ar), 4.92 (d, J=3.5 Hz, 1H, H-1′), 4.89, 4.55 (ABq, J=11.3 Hz, 2H, CH₂Ar), 4.86, 4.67 (ABq, J=11.3 Hz, 2H, CH₂Ar), 4.65-4.59 (m, 2H, CH₂Ar), 4.60-4.58 (m, 1H, H-4), 4.52 (dd, J=12.0, 2.2 Hz, 1H, H-6′a), 4.44 (dd, J=12.0, 4.6 Hz, 1H, H-6′b), 4.31 (ddd, J=9.8, 9.7, 3.5 Hz, 1H, H-2′), 4.00 (d, J=7.7 Hz, 1H, H-6a), 3.90-3.87 (m, 1H, H-5′), 3.76-3.69 (m, 4H, H-3′, H-4′, H-3, H-5), 3.67 (dd, J=7.7, 5.0 Hz, 1H, H-6b), 3.51 (dd, J=6.3, 1.5 Hz, 1H, H-2), 1.36 (s, 3H, CH₃); ¹³C NMR (125 MHz, CDCl₃) δ 170.1 (C), 166.1 (C), 138.1 (C), 138.0 (C), 137.5 (C), 134.6 (C), 133.2 (CH), 133.1 (C), 129.5 (CH), 128.64 (CH), 128.57 (CH), 128.48 (CH), 128.46 (CH), 128.13 (CH), 128.07 (CH), 128.05 (CH), 128.01 (CH), 127.99 (CH), 127.9 (CH), 127.7 (CH), 127.5 (CH), 127.3 (CH), 126.2 (CH), 126.1 (CH), 126.0 (CH), 100.2 (CH), 99.2 (CH), 82.8 (CH), 80.6 (CH), 80.4 (CH), 80.0 (CH), 77.7 (CH), 75.4 (CH₂), 75.3 (CH₂), 75.2 (CH₂), 73.8 (CH), 72.7 (CH₂), 70.7 (CH), 65.3 (CH₂), 63.2 (CH₂), 52.6 (CH), 22.7 (CH₃). 61: ¹H NMR (500 MHz, CDCl₃) δ 7.94 (dd, J=8.3, 1.1 Hz, 2H, BzH), 7.68-7.66 (m, 1H, ArH), 7.60-7.58 (m, 1H, ArH), 7.55-7.50 (m, 2H, ArH), 7.40-7.28 (m, 19H, ArH), 7.22-7.19 (m, 1H, ArH), 7.10 (dd, J=8.4, 1.5 Hz, 1H, ArH), 5.68 (d, J=9.7 Hz, 1H, NH), 5.55 (dd, J=11.0, 8.8 Hz, 1H, H-3′), 5.32 (d, J=1.2 Hz, 1H, H-1), 5.05 (d, J=3.7 Hz, 1H, H-1′), 4.94, 4.68 (ABq, J=11.0 Hz, 2H, CH₂Ar), 4.69-4.64 (m, 3H, CH₂Ar, H-5), 4.65, 4.59 (ABq, J=11.0 Hz, 2H, CH₂Ar), 4.60, 4.48 (ABq, J=12.0 Hz, 2H, CH₂Ar), 4.47 (ddd, J=11.0, 9.7, 3.7 Hz, 1H, H-2′), 4.06 (d, J=7.8 Hz, 1H, H-6a), 3.90 (dd, J=9.8, 8.8 Hz, 1H, H-4′), 3.85 (ddd, J=9.8, 4.0, 1.4 Hz, 1H, H-5′), 3.78 (dd, J=8.5, 7.5 Hz, 1H, H-3), 3.76-3.73 (m, 1H, H-4), 3.75 (dd, J=10.5, 4.0 Hz, 1H, H-6′a), 3.70 (dd, J=7.8, 5.1 Hz, 1H, H-6b), 3.65 (dd, J=10.5, 1.4 Hz, 1H, H-6′b), 3.52 (dd, J=7.5, 1.2 Hz, 1H, H-2), 1.19 (s, 3H, CH₃); ¹³C NMR (125 MHz, CDCl₃) δ 170.2 (C), 166.7 (C), 138.1 (C), 137.7 (C), 137.6 (C), 134.6 (C), 133.3 (CH), 133.0 (C), 132.9 (C), 129.7 (CH), 129.4 (C), 128.63 (CH), 128.55 (CH), 128.46 (CH), 128.45 (CH), 128.2 (CH), 128.0 (CH), 127.9 (CH), 127.83 (CH), 127.80 (CH), 127.6 (CH), 127.0 (CH), 126.0 (CH), 125.9 (CH), 99.9 (CH), 99.3 (CH), 82.9 (CH), 80.8 (CH), 80.3 (CH), 75.9 (CH), 75.6 (CH₂), 75.1 (CH₂), 73.8 (CH), 73.7 (CH), 73.6 (CH₂), 72.7 (CH₂), 72.1 (CH), 68.4 (CH₂), 65.5 (CH₂), 52.1 (CH), 22.2 (CH₃); HRMS (FAB, MH⁺) calcd for C₅₃H₅₄NO₁₁ 880.3697, found 880.3698. 62: ¹H NMR (400 MHz, CDCl₃) δ 7.83-7.73 (m, 3H, ArH), 7.59 (s, 1H, ArH), 7.49-7.44 (m, 2H, ArH), 7.34-7.20 (m, 21H, ArH), 5.30 (d, J=1.4 Hz, 1H, H-1), 5.25 (d, J=9.4 Hz, 1H, NH), 4.95-4.90 (m, 2H, H-1′, CH₂Ar), 4.87 (d, J=11.3 Hz, 1H, CH₂Ar), 4.83 (d, J=11.5 Hz, 1H, CH₂Ar), 4.70-4.58 (m, 5H, H-3, CH₂Ar), 4.55 (d, J=11.3 Hz, 1H, CH₂Ar), 4.52, 4.45 (ABq, J=12.1 Hz, 2H, CH₂Ar), 4.26 (td, J=9.4, 3.7 Hz, 1H, H-2′), 3.99 (d, J=7.7 Hz, 1H, H-6a), 3.75-3.58 (m, 8H, H-4, H-5, H-6b, H-3′, H-4′, H-5′, H-6′a, H-6′b), 3.54-3.47 (m, 1H, H-2), 1.34 (s, 3H, CH₃); ¹³C NMR (100 MHz, CDCl₃) δ 170.0 (C), 138.2 (C), 137.7 (C), 137.5 (C), 135.1 (C), 133.2 (C), 133.0 (C), 128.6 (CH), 128.54 (CH), 128.51 (CH), 128.4 (CH), 128.3 (CH), 128.1 (CH), 128.0 (CH), 127.93 (CH), 127.88 (CH), 127.8 (CH), 127.72 (CH), 127.66 (CH), 127.4 (CH), 127.0 (CH), 126.2 (CH), 126.1 (CH), 126.0 (CH), 100.2 (CH), 99.2 (CH), 82.8 (CH), 80.7 (CH), 80.25 (CH), 80.18 (CH), 78.2 (CH), 75.3 (CH₂), 75.1 (CH₂), 74.9 (CH₂), 73.8 (CH), 73.5 (CH₂), 72.7 (CH₂), 72.2 (CH), 68.6 (CH₂), 65.5 (CH₂), 52.5 (CH), 22.7 (CH₃).

Synthesis of Compounds 63-78: To a mixture of the disaccharide (31-46, 1.0 equiv) in a mixed solvent MeOH and CH₂Cl₂ (½ ratio, 10 μL per 1 mg of the disaccharide) was added a 1.0 M solution of triethylphosphine in THF (10 equiv) at room temperature. The mixture was stirred for 8 h, and the solvent was evaporated at reduced pressure to afford the crude amine. This amine was dissolved in acetone (1 mL per 50 mg disaccharide) at room temperature, saturated Na₂CO₃ (aq) (0.25 mL per 50 mg of the disaccharide), and CbzCl (1.5 equiv) were sequentially added to the solution, and the mixture was kept stirring for another 2 h. The mixture was filtered through celite, the salt was washed by dichloromethane, and the filtrate was concentrated in vacuo. The residue was purified by flash column chromatography on silica gel to give the corresponding N-Cbz derivative. The yields of compounds 63-78 are summarized in Scheme 4. 63: ¹H NMR (500 MHz, CDCl₃) δ 8.03-7.98 (m, 4H, ArH), 7.91-7.88 (m, 2H, ArH), 7.74-7.66 (m, 4H, ArH), 7.64-7.49 (m, 6H, ArH), 7.48-7.19 (m, 19H, ArH), 7.10-7.04 (m, 3H, ArH), 7.03-6.87 (m, 8H, ArH), 5.71 (d, J=10.3 Hz, 1H, NH), 5.64 (dd, J=10.7, 9.5 Hz, 1H, H-3′), 5.45 (d, J=3.5 Hz, 1H, H-1′), 5.35 (dd, J=10.1, 8.8 Hz, 1H, H-2), 4.75 (d, J=10.1 Hz, 1H, H-1), 4.73-4.69 (m, 2H, CH₂Ar), 4.66-4.60 (m, 4H, CH₂Ar), 4.33 (dd, J=12.0, 4.0 Hz, 1H, H-6′a), 4.23 (dd, J=12.0, 1.8 Hz, 1H, H-6′b), 4.18 (ddd, J=10.7, 10.3, 3.5 Hz, 1H, H-2′), 4.13-4.05 (m, 2H, H-4, H-6a), 4.04-4.01 (m, 1H, H-5′), 3.97 (dd, J=11.3, 1.0 Hz, 1H, H-6b), 3.92 (t, J=8.8 Hz, 1H, H-3), 3.87 (t, J=9.5 Hz, 1H, H-4′), 3.48-3.43 (m, 1H, H-5), 2.27 (s, 3H, CH₃), 1.03 (s, 9H, t-Bu); ¹³C NMR (125 MHz, CDCl₃) δ 166.3 (C), 166.0 (C), 165.5 (C), 156.1 (C), 137.8 (C), 136.5 (C), 136.3 (C), 135.9 (CH), 135.5 (CH), 134.4 (C), 133.3 (CH), 133.2 (CH), 133.1 (CH), 133.0 (C), 132.7 (C), 132.4 (CH), 129.93 (C), 129.86 (CH), 129.8 (CH), 129.7 (C), 128.4 (CH), 128.3 (CH), 128.10 (CH), 128.07 (CH), 128.0 (CH), 127.8 (CH), 127.7 (CH), 127.6 (CH), 127.4 (CH), 127.3 (CH), 126.1 (CH), 126.0 (CH), 125.9 (CH), 99.2 (CH), 87.4 (CH), 83.9 (CH), 80.0 (CH), 76.0 (CH), 75.4 (CH), 75.0 (CH₂), 74.8 (CH₂), 73.9 (CH), 73.0 (CH), 70.6 (CH), 66.4 (CH₂), 63.1 (CH₂×2), 54.5 (CH), 26.8 (t-Bu), 21.1 (CH₃), 19.3 (C); HRMS (FAB, MH⁺) calcd for C₈₂H₈₀NO₁₄SSi 1362.5069, found 1362.5045. 64: ¹H NMR (400 MHz, CDCl₃) δ 8.04 (dd, J=8.4, 1.2 Hz, 2H, ArH), 7.91 (dd, J=8.2, 1.2 Hz, 2H, ArH), 7.73-7.63 (m, 5H, ArH), 7.60-7.47 (m, 4H, ArH), 7.46-7.18 (m, 21H, ArH), 7.12-6.94 (m, 10H, ArH), 6.90-6.85 (m, 2H, ArH), 5.51 (d, J=3.6 Hz, 1H, H-1′), 5.50 (d, J=10.6 Hz, 1H, NH), 5.46 (dd, J=10.6, 9.6 Hz, 1H, H-3′), 5.34 (dd, J=10.1, 8.8 Hz, 1H, H-2), 4.77 (d, J=10.1 Hz, 1H, H-1), 4.72 (d, J=12.4 Hz, 1H, CH₂Ar), 4.62-4.49 (m, 6H, CH₂Ar), 4.27 (d, J=12.0 Hz, 1H, CH₂Ar), 4.17 (td, J=10.6, 3.6 Hz, 1H, H-2′), 4.08-3.98 (m, 3H, H-4, H-6a, H-6b), 3.93 (t, J=9.6 Hz, 1H, H-4′), 3.88 (t, J=8.8 Hz, 1H, H-3), 3.78-3.71 (m, 1H, H-5′), 3.49-3.45 (m, 1H, H-5), 3.43 (dd, J=11.0, 2.4 Hz, 1H, H-6′a), 3.29 (dd, J=11.0, 1.6 Hz, 1H, H-6′b), 2.26 (s, 3H, CH₃), 1.05 (s, 9H, t-Bu); ¹³C NMR (100 MHz, CDCl₃) δ 166.4 (C), 165.2 (C), 155.8 (C), 137.9 (C), 137.6 (C), 136.6 (C), 136.2 (C), 135.9 (CH), 135.6 (CH), 135.1 (C), 133.7 (C), 133.3 (CH), 133.0 (CH), 132.9 (C), 132.1 (CH), 130.3 (C), 129.84 (CH), 129.79 (CH), 129.7 (C), 129.64 (CH), 129.56 (CH), 128.44 (CH), 128.38 (CH), 128.3 (CH), 128.14 (CH), 128.10 (CH), 128.03 (CH), 127.95 (CH), 127.9 (CH), 127.8 (CH), 127.7 (CH), 127.63 (CH), 127.61 (CH), 127.6 (CH), 127.5 (CH), 126.9 (CH), 126.2 (CH), 125.9 (CH), 125.8 (CH), 98.8 (CH), 87.4 (CH), 84.4 (CH), 79.9 (CH), 75.4 (CH), 74.73 (CH₂), 74.65 (CH₂), 74.6 (CH), 74.0 (CH), 73.5 (CH₂), 73.0 (CH), 72.1 (CH), 68.0 (CH₂), 66.4 (CH₂), 63.3 (CH₂), 54.2 (CH), 26.9 (t-Bu), 21.1 (CH₃), 19.4 (C); HRMS (FAB, MH⁺) calcd for C₈₂H₈₂NO₁₃SSi 1348.5276, found 1348.5264. 65: ¹H NMR (400 MHz, CDCl₃) δ 7.99 (d, J=7.5 Hz, 2H, ArH), 7.85 (dd, J=8.3, 1.2 Hz, 2H, ArH), 7.77-7.64 (m, 8H, ArH), 7.61 (s, 1H, ArH), 7.55 (t, J=7.5 Hz, 1H, ArH), 7.48 (t, J=7.5 Hz, 1H, ArH), 7.44-7.38 (m, 4H, ArH), 7.37-7.14 (m, 20H, ArH), 7.09-7.04 (m, 2H, ArH), 6.98-6.93 (m, 5H, ArH), 5.69 (d, J=10.2 Hz, 1H, NH), 5.38-5.31 (m, 2H, H-2, H-1′), 5.08, 4.93 (ABq, J=12.3 Hz, 2H, CH₂Ar), 4.92 (d, J=10.9 Hz, 1H, CH₂Ar), 4.72 (d, J=9.9 Hz, 1H, H-1), 4.69 (d, J=10.9 Hz, 1H, CH₂Ar), 4.64, 4.58 (ABq, J=11.0 Hz, 2H, CH₂Ar), 4.58, 4.38 (ABq, J=11.0 Hz, 2H, CH₂Ar), 4.31 (dd, J=11.5, 3.6 Hz, 1H, H-6′a), 4.27 (dd, J=11.5, 2.4 Hz, 1H, H-6′b), 4.10-4.01 (m, 3H, H-4, H-6a, H-2′), 3.96-3.90 (m, 2H, H-6b, H-5′), 3.88 (t, J=9.2 Hz, 1H, H-3), 3.70 (t, J=9.5 Hz, 1H, H-4′), 3.59 (t, J=9.5 Hz, 1H, H-3′), 3.42-3.35 (m, 1H, H-5), 2.26 (s, 3H, CH₃), 1.02 (s, 9H, t-Bu); ¹³C NMR (125 MHz, CDCl₃) δ 166.0 (C), 165.1 (C), 156.1 (C), 138.1 (C), 137.8 (C), 136.6 (C), 135.9 (CH), 135.5 (CH), 135.0 (C), 133.3 (CH), 133.2 (C), 133.1 (C), 133.03 (C), 132.99 (CH), 132.8 (C), 132.5 (CH), 129.9 (C), 129.8 (CH), 129.71 (C), 129.65 (CH), 129.61 (CH), 129.5 (CH), 128.4 (CH), 128.33 (CH), 128.27 (CH), 128.24 (CH), 128.18 (CH), 128.0 (CH), 127.9 (CH), 127.7 (CH), 127.5 (CH), 127.2 (CH), 126.1 (CH), 126.0 (CH), 99.2 (CH), 87.4 (CH), 83.9 (CH), 81.3 (CH), 80.2 (CH), 77.3 (CH), 75.7 (CH), 75.2 (CH₂), 75.1 (CH₂), 74.6 (CH₂), 72.8 (CH), 70.6 (CH), 66.7 (CH₂), 63.2 (CH₂), 63.0 (CH₂), 55.6 (CH), 26.8 (t-Bu), 21.1 (CH₃), 19.3 (C); HRMS (FAB, MNa⁺) calcd for C₈₂H₈₁NO₁₃SSiNa 1370.5096, found 1370.5085. 66: ¹H NMR (500 MHz, CDCl₃) δ 8.00 (d, J=7.7 Hz, 2H, ArH), 7.83-7.68 (m, 9H, ArH), 7.58-7.54 (m, 2H, ArH), 7.49-7.41 (m, 5H, ArH), 7.33-7.15 (m, 19H, ArH), 7.07-6.94 (m, 9H, ArH), 5.41 (d, J=3.6 Hz, 1H, H-1′), 5.39 (d, J=10.5 Hz, 1H, NH), 5.34 (dd, J=9.9, 8.8 Hz, 1H, H-2), 5.05, 4.91 (ABq, J=12.2 Hz, 2H, CH₂Ar), 4.87 (d, J=10.8 Hz, 1H, CH₂Ar), 4.76 (d, J=9.9 Hz, 1H, H-1), 4.64 (d, J=11.4 Hz, 1H, CH₂Ar), 4.63-4.52 (m, 3H, CH₂Ar), 4.50-4.43 (m, 2H, CH₂Ar), 4.27 (d, J=12.1 Hz, 1H, CH₂Ar), 4.08-4.02 (m, 3H, H-4, H-6a, H-2′), 4.00 (dd, J=11.4, 1.6 Hz, 1H, H-6b), 3.86 (t, J=8.8 Hz, 1H, H-3), 3.74 (dd, J=9.5, 8.8 Hz, 1H, H-4′), 3.71-3.67 (m, 1H, H-5′), 3.54-3.46 (m, 2H, H-3′, H-6′a), 3.45-3.41 (m, 1H, H-5), 3.35 (d, J=10.5 Hz, 1H, H-6′b), 2.27 (s, 3H, CH₃), 1.06 (s, 9H, t-Bu); ¹³C NMR (125 MHz, CDCl₃) δ 165.1 (C), 155.9 (C), 138.4 (C), 138.0 (C), 137.6 (C), 136.7 (C), 136.5 (C), 135.9 (CH), 135.7 (C), 135.6 (CH), 133.5 (C), 133.2 (CH), 133.1 (C), 133.0 (C), 132.2 (CH), 130.2 (C), 129.82 (CH), 129.75 (C), 129.6 (CH), 128.4 (CH), 128.32 (CH), 128.26 (CH), 128.2 (CH), 128.0 (CH), 127.9 (CH), 127.7 (CH), 127.6 (CH), 127.4 (CH), 126.7 (CH), 126.1 (CH), 126.0 (CH), 125.9 (CH), 98.9 (CH), 87.3 (CH), 84.2 (CH), 81.1 (CH), 80.1 (CH), 77.7 (CH), 74.9 (CH₂×2), 74.6 (CH), 74.4 (CH₂), 73.4 (CH₂), 72.9 (CH), 72.3 (CH), 68.4 (CH₂), 66.7 (CH₂), 63.3 (CH₂), 55.1 (CH), 26.9 (t-Bu), 21.1 (CH₃), 19.4 (C); HRMS (FAB, MNa⁺) calcd for C₈₂H₈₃NO₁₂SSiNa 1356.5303, found 1356.5289. 67: ¹H NMR (400 MHz, CDCl₃) δ 7.97 (dd, J=8.2, 1.2 Hz, 2H, ArH), 7.90 (dd, J=8.4, 1.1 Hz, 2H, ArH), 7.73-7.67 (m, 4H, ArH), 7.63-7.48 (m, 5H, ArH), 7.47-7.42 (m, 3H, ArH), 7.41-7.17 (m, 20H, ArH), 7.13-7.06 (m, 4H, ArH), 7.04-6.98 (m, 4H, ArH), 6.92-6.87 (m, 2H, ArH), 5.71 (d, J=10.7 Hz, 1H, NH), 5.60 (dd, J=10.8, 9.3 Hz, 1H, H-3′), 5.49 (d, J=3.5 Hz, 1H, H-1′), 4.97-4.93 (m, 2H, CH₂Ar), 4.75 (d, J=10.9 Hz, 1H, CH₂Ar), 4.73 (d, J=12.3 Hz, 1H, CH₂Ar), 4.66 (d, J=9.7 Hz, 1H, H-1), 4.64-4.58 (m, 4H, CH₂Ar), 4.30 (dd, J=12.2, 3.4 Hz, 1H, H-6′a), 4.23-4.15 (m, 2H, H-2′, H-6′b), 4.05 (dd, J=11.5, 4.3 Hz, 1H, H-6a), 4.00-3.92 (m, 3H, H-4, H-6b, H-5′), 3.86 (t, J=9.3 Hz, 1H, H-4′), 3.68 (t, J=8.7 Hz, 1H, H-3), 3.55 (dd, J=9.7, 8.7 Hz, 1H, H-2), 3.41-3.36 (m, 1H, H-5), 2.29 (s, 3H, CH₃), 1.04 (s, 9H, t-Bu); ¹³C NMR (125 MHz, CDCl₃) δ 166.2 (C), 166.0 (C), 156.0 (C), 137.8 (C), 137.5 (C), 137.4 (C), 136.3 (C), 135.8 (CH), 135.5 (CH), 134.4 (C), 133.2 (C), 133.1 (CH), 133.02 (CH), 132.95 (C), 132.8 (C), 131.8 (CH), 130.6 (C), 129.9 (CH), 129.7 (CH), 129.62 (CH), 129.57 (CH), 128.4 (CH), 128.3 (CH), 128.1 (CH), 127.83 (CH), 127.81 (CH), 127.7 (CH), 127.6 (CH), 127.50 (CH), 127.48 (CH), 127.41 (CH), 127.37 (CH), 126.1 (CH), 125.94 (CH), 125.89 (CH), 98.9 (CH), 88.4 (CH), 85.4 (CH), 81.3 (CH), 79.7 (CH), 75.6 (CH), 75.4 (CH), 75.1 (CH₂), 74.9 (CH₂), 74.8 (CH₂), 73.9 (CH), 70.5 (CH), 66.4 (CH₂), 63.3 (CH₂), 63.1 (CH₂), 54.4 (CH), 26.9 (t-Bu), 21.1 (CH₃), 19.3 (C); HRMS (FAB, MH⁺) calcd for C₈₂H₈₂NO₁₃SSi 1348.5276, found 1348.5264. 68: ¹H NMR (500 MHz, CDCl₃) δ 7.85 (dd, J=8.3, 1.1 Hz, 2H, ArH), 7.75-7.65 (m, 8H, ArH), 7.59 (s, 1H, ArH), 7.50-7.37 (m, 5H, ArH), 7.35-7.17 (m, 23H, ArH), 7.15-7.07 (m, 5H, ArH), 7.00 (d, J=8.0 Hz, 2H, ArH), 5.87 (d, J=10.2 Hz, 1H, NH), 5.34 (d, J=3.3 Hz, 1H, H-1′), 5.08 (d, J=12.2 Hz, 1H, CH₂Ar), 5.00-4.85 (m, 4H, CH₂Ar), 4.67 (m, 2H, CH₂Ar), 4.62 (d, J=9.5 Hz, 1H, H-1), 4.58-4.48 (m, 2H, CH₂Ar), 4.34-4.23 (m, 3H, CH₂Ar, H-6′a, H-6′b), 4.09-4.00 (m, 2H, H-6a, H-2′), 3.95 (t, J=8.9 Hz, 1H, H-4), 3.93-3.87 (m, 2H, H-6b, H-5′), 3.70 (t, J=9.4 Hz, 1H, H-4′), 3.62 (t, J=8.9 Hz, 1H, H-3), 3.60-3.52 (m, 2H, H-2, H-3′), 3.35-3.27 (m, 1H, H-5), 2.28 (s, 3H, CH₃), 1.03 (s, 9H, t-Bu); ¹³C NMR (100 MHz, CDCl₃) δ 166.0 (C), 156.1 (C), 138.2 (C), 137.7 (C), 137.5 (C), 136.7 (C), 135.8 (CH), 135.6 (CH), 135.0 (C), 133.1 (C), 133.01 (C), 132.95 (CH), 131.9 (CH), 130.6 (C), 129.72 (CH), 129.67 (C), 129.64 (CH), 129.61 (CH), 129.5 (CH), 128.42 (CH), 128.37 (CH), 128.31 (CH), 128.26 (CH), 128.2 (CH), 128.0 (CH), 127.9 (CH), 127.7 (CH), 127.6 (CH), 127.53 (CH), 127.47 (CH), 127.2 (CH), 126.8 (CH), 126.1 (CH), 126.0 (CH), 125.9 (CH), 99.1 (CH), 88.2 (CH), 85.0 (CH), 81.4 (CH), 80.9 (CH), 80.0 (CH), 77.2 (CH), 75.6 (CH), 75.3 (CH₂), 75.1 (CH₂), 75.0 (CH₂), 74.8 (CH₂), 70.6 (CH), 66.7 (CH₂), 63.2 (CH₂), 63.1 (CH₂), 55.7 (CH), 26.8 (t-Bu), 21.1 (CH₃), 19.3 (C); HRMS (FAB, MH⁺) calcd for C₈₂H₈₄NO₁₂SSi 1334.5484, found 1334.5519. 69: ¹H NMR (400 MHz, CDCl₃) δ 7.88 (dd, J=8.1, 0.9 Hz, 2H, ArH), 7.72-7.63 (m, 7H, ArH), 7.59-7.55 (m, 2H, ArH), 7.52-7.46 (m, 3H, ArH), 7.44-7.36 (m, 5H, ArH), 7.34-7.11 (m, 17H, ArH), 7.10-6.94 (m, 8H, ArH), 6.89-6.84 (m, 2H, ArH), 5.53 (d, J=3.6 Hz, 1H, H-1′), 5.45 (d, J=10.2 Hz, 1H, NH), 5.44 (dd, J=10.8, 9.3 Hz, 1H, H-3′), 4.95 (d, J=10.4 Hz, 1H, CH₂Ar), 4.89 (d, J=11.0 Hz, 1H, CH₂Ar), 4.70 (d, J=12.4 Hz, 1H, CH₂Ar), 4.67 (d, J=9.8 Hz, 1H, H-1), 4.62 (d, J=11.0 Hz, 1H, CH₂Ar), 4.61 (d, J=10.4 Hz, 1H, CH₂Ar), 4.57-4.48 (m, 4H, CH₂Ar), 4.25 (d, J=12.1 Hz, 1H, CH₂Ar), 4.17 (ddd, J=10.8, 10.2, 3.6 Hz, 1H, H-2′), 4.01-3.87 (m, 4H, H-4, H-6a, H-6b, H-4′), 3.73-3.68 (m, 1H, H-5′), 3.64 (t, J=8.5 Hz, 1H, H-3), 3.52 (dd, J=9.8, 8.5 Hz, 1H, H-2), 3.42 (dd, J=10.7, 2.6 Hz, 1H, H-6′a), 3.41-3.37 (m, 1H, H-5), 3.26 (dd, J=10.7, 1.2 Hz, 1H, H-6′b), 2.29 (s, 3H, CH₃), 1.04 (s, 9H, t-Bu); ¹³C NMR (100 MHz, CDCl₃) δ 166.5 (C), 155.8 (C), [symbol] (C), 137.8 (C), 137.5 (C), 137.3 (C), 136.2 (C), 135.8 (CH), 135.6 (CH), 135.1 (C), 133.7 (C), 133.02 (C), 132.97 (CH), 132.95 (C), 132.8 (C), 131.6 (CH), 130.8 (C), 129.8 (CH), 129.75 (CH), 129.70 (C), 129.63 (CH), 129.55 (CH), 128.4 (CH), 128.3 (CH), 128.1 (CH), 127.9 (CH), 127.8 (CH), 127.7 (CH), 127.64 (CH), 127.58 (CH), 127.52 (CH), 127.49 (CH), 126.9 (CH), 126.2 (CH), 125.9 (CH), 125.7 (CH), 98.7 (CH), 88.4 (CH), 85.7 (CH), 81.3 (CH), 79.6 (CH), 75.5 (CH), 74.9 (CH₂×2), 74.6 (CH₂), 74.4 (CH), 74.1 (CH), 73.5 (CH₂), 71.9 (CH), 68.0 (CH₂), 66.4 (CH₂), 63.4 (CH₂), 54.1 (CH), 26.9 (t-Bu), 21.1 (CH₃), 19.4 (C); HRMS (FAB, MH⁺) calcd for C₈₂H₈₄NO₁₂SSi 1334.5484, found 1334.5513. 70: ¹H NMR (400 MHz, CDCl₃) δ 7.83-767 (m, 10H, ArH), 7.53-7.40 (m, 7H, ArH), 7.32-7.11 (m, 26H, ArH), 7.02-6.97 (m, 3H, ArH), 5.59 (d, J=10.2 Hz, 1H, NH), 5.37 (d, J=3.5 Hz, 1H, H-1′), 5.05 (d, J=12.2 Hz, 1H, CH₂Ar), 4.93-4.82 (m, 4H, CH₂Ar), 4.64 (d, J=9.4 Hz, 1H, H-1), 4.62-4.55 (m, 4H, CH₂Ar), 4.49, 4.27 (ABq, J=12.0 Hz, 2H, CH₂Ar), 4.38 (d, J=11.2 Hz, 1H, CH₂Ar), 4.05 (td, J=10.2, 3.5 Hz, 1H, H-2′), 4.03-3.87 (m, 3H, H-4, H-6a, H-6b), 3.74 (t, J=9.7 Hz, 1H, H-4′), 3.71-3.65 (m, 1H, H-5′), 3.60 (t, J=8.2 Hz, 1H, H-3), 3.56-3.46 (m, 3H, H-2, H-3′, H-6′a), 3.40-3.31 (m, 2H, H-5, H-6′b), 2.28 (s, 3H, CH₃), 1.05 (s, 9H, t-Bu); ¹³C NMR (100 MHz, CDCl₃) δ 155.9 (C), 138.4 (C), 138.0 (C), 137.7 (C), 137.5 (C), 137.3 (C), 136.6 (C), 135.8 (CH), 135.7 (C), 135.6 (CH), 133.4 (C), 133.2 (C), 133.1 (C), 132.9 (C), 131.7 (CH), 130.7 (C), 129.7 (CH), 129.6 (CH), 128.4 (CH), 128.3 (CH), 128.24 (CH), 128.22 (CH), 128.17 (CH), 128.0 (CH), 127.9 (CH), 127.73 (CH), 127.70 (CH), 127.66 (CH), 127.58 (CH), 127.4 (CH), 127.0 (CH), 126.7 (CH), 126.1 (CH), 126.0 (CH), 125.9 (CH), 98.9 (CH), 88.1 (CH), 85.1 (CH), 81.2 (CH), 80.8 (CH), 80.0 (CH), 77.7 (CH), 75.0 (CH₂×2), 74.9 (CH₂), 74.57 (CH), 74.56 (CH₂), 73.4 (CH₂), 72.2 (CH), 68.4 (CH₂), 66.7 (CH₂), 63.4 (CH₂), 55.2 (CH), 26.9 (t-Bu), 21.1 (CH₃), 19.4 (C); HRMS (FAB, MH⁺) calcd for C₈₂H₈₆NO₁₁SSi 1320.5691, found 1320.5724. 71: ¹H NMR (400 MHz, CDCl₃) δ 8.04-7.98 (m, 6H, BzH), 7.67-7.56 (m, 6H, ArH), 7.52 (s, 1H, ArH), 7.47-7.37 (m, 8H, ArH), 7.14-7.02 (m, 9H, ArH), 6.94-6.92 (m, 2H, ArH), 5.65 (dd, J=10.6, 9.5 Hz, 1H, H-3′), 5.52 (d, J=1.7 Hz, 1H, H-1), 5.26 (d, J=3.7 Hz, 1H, H-1′), 5.16 (d, J=10.6 Hz, 1H, NH), 5.06 (dd, J=8.0, 1.7 Hz, 1H, H-2), 4.87, 4.63 (ABq, J=12.4 Hz, 2H, CH₂Ar), 4.75 (s, 2H, CH₂Ar), 4.71-4.67 (m, 1H, H-5), 4.69, 4.66 (ABq, J=10.7 Hz, 2H, CH₂Ar), 4.60 (dd, J=12.0, 2.3 Hz, 1H, H-6′a), 4.57 (dd, J=12.0, 4.5 Hz, 1H, H-6′b), 4.50 (td, J=10.6, 3.7 Hz, 1H, H-2′), 4.18 (d, J=7.8 Hz, 1H, H-6a), 4.07 (ddd, J=9.5, 4.5, 2.3 Hz, 1H, H-5′), 4.05-4.01 (m, 2H, H-3, H-4), 3.92 (t, J=9.5 Hz, 1H, H-4′), 3.79 (dd, J=7.8, 4.8 Hz, 1H, H-6b); ¹³C NMR (125 MHz, CDCl₃) δ 166.5 (C), 166.1 (C), 165.6 (C), 155.9 (C), 137.3 (C), 135.9 (C), 134.0 (C), 133.44 (CH), 133.42 (CH), 133.3 (CH), 133.04 (C), 132.99 (C), 129.9 (CH), 129.8 (CH), 129.6 (CH), 129.5 (C), 129.31 (C), 129.29 (C), 128.55 (CH), 128.51 (CH), 128.46 (CH), 128.4 (CH), 128.2 (CH), 127.8 (CH), 127.7 (CH), 127.64 (CH), 127.61 (CH), 127.57 (CH), 127.4 (CH), 126.10 (CH), 126.07 (CH), 125.9 (CH), 99.9 (CH), 99.3 (CH), 79.4 (CH), 79.1 (CH), 77.0 (CH), 75.7 (CH), 75.2 (CH₂), 75.1 (CH₂), 74.1 (CH), 73.7 (CH), 70.4 (CH), 66.8 (CH₂), 65.5 (CH₂), 63.2 (CH₂), 54.1 (CH); HRMS (FAB, MH⁺) calcd for C₅₉H₅₄NO₁₄ 1000.3544, found 1000.3555. 72: ¹H NMR (500 MHz, CDCl₃) δ 8.01 (dd, J=8.4, 1.1 Hz, 2H, BzH), 7.96 (dd, J=8.4, 1.1 Hz, 2H, BzH), 7.69-7.67 (m, 1H, ArH), 7.62-7.60 (m, 1H, ArH), 7.58-7.53 (m, 3H, ArH), 7.44-7.28 (m, 12H, ArH), 7.12-7.02 (m, 9H, ArH), 6.94-6.92 (m, 2H, ArH), 5.54 (dd, J=10.4, 9.4 Hz, 1H, H-3′), 5.50 (d, J=1.7 Hz, 1H, H-1), 5.25 (d, J=3.7 Hz, 1H, H-1′), 5.11 (d, J=10.4 Hz, 1H, NH), 5.06 (dd, J=8.2, 1.7 Hz, 1H, H-2), 4.87 (d, J=12.4 Hz, 1H, CH₂Ar), 4.70 (dd, J=5.1, 4.0 Hz, 1H, H-5), 4.68, 4.64 (ABq, J=11.0 Hz, 2H, CH₂Ar), 4.66, 4.61 (ABq, J=10.8 Hz, 2H, CH₂Ar), 4.63, 4.52 (ABq, J=12.1 Hz, 2H, CH₂Ar), 4.62 (d, J=12.4 Hz, 1H, CH₂Ar), 4.23 (td, J=10.4, 3.7 Hz, 1H, H-2′), 4.14 (d, J=7.8 Hz, 1H, H-6a), 4.03-4.00 (m, 2H, H-3, H-4), 3.92 (t, J=9.4 Hz, 1H, H-4′), 3.84 (ddd, J=9.4, 4.2, 1.5 Hz, 1H, H-5′), 3.77 (dd, J=10.7, 4.2 Hz, 1H, H-6′a), 3.75 (dd, J=7.8, 5.1 Hz, 1H, H-6b), 3.70 (dd, J=10.7, 1.5 Hz, 1H, H-6′b); ¹³C NMR (125 MHz, CDCl₃) δ 166.6 (C), 165.6 (C), 155.8 (C), 137.6 (C), 137.4 (C), 136.0 (C), 134.5 (C), 133.4 (CH), 133.3 (CH), 133.0 (C), 132.9 (C), 129.84 (CH), 129.82 (CH), 129.4 (C), 129.3 (C), 128.5 (CH), 128.43 (CH), 128.41 (CH), 128.35 (CH), 128.21 (CH), 128.19 (CH), 127.9 (CH), 127.84 (CH), 127.78 (CH), 127.71 (CH), 127.66 (CH), 127.6 (CH), 127.5 (CH), 127.1 (CH), 126.03 (CH), 125.96 (CH), 100.0 (CH), 99.2 (CH), 79.3 (CH×2), 77.0 (CH), 75.8 (CH), 75.1 (CH₂×2), 74.1 (CH), 73.8 (CH), 73.7 (CH₂), 71.9 (CH), 68.3 (CH₂), 66.7 (CH₂), 65.6 (CH₂), 54.1 (CH); HRMS (FAB, MH⁺) calcd for C₅₉H₅₆NO₁₃ 986.3752, found 986.3762. 74: ¹H NMR (500 MHz, CDCl₃) δ 8.00 (d, J=7.5 Hz, 2H, BzH), 7.82-7.74 (m, 3H, ArH), 7.58-7.55 (m, 2H, ArH), 7.49-7.41 (m, 4H, ArH), 7.36-7.20 (m, 17H, ArH), 7.12-7.06 (m, 4H, ArH), 5.49 (d, J=1.6 Hz, 1H, H-1), 5.13, 4.84 (ABq, J=12.2 Hz, 2H, CH₂Ar), 5.12 (d, J=3.6 Hz, 1H, H-1′), 5.05 (dd, J=8.0, 1.6 Hz, 1H, H-2), 4.93, 4.64 (ABq, J=10.8 Hz, 2H, CH₂Ar), 4.80, 4.67 (ABq, J=11.2 Hz, 2H, CH₂Ar), 4.78 (d, J=9.8 Hz, 1H, NH), 4.77-4.73 (m, 1H, H-5), 4.59, 4.56 (ABq, J=11.3 Hz, 2H, CH₂Ar), 4.55, 4.48 (ABq, J=12.2 Hz, 2H, CH₂Ar), 4.06 (td, J=9.8, 3.6 Hz, 1H, H-2′), 4.04 (d, J=7.6 Hz, 1H, H-6a), 3.97-3.90 (m, 2H, H-3, H-4), 3.71 (dd, J=7.6, 5.0 Hz, 1H, H-6b), 3.69-3.58 (m, 5H, H-3′, H-4′, H-5′, H-6′a, H-6′b); ¹³C NMR (125 MHz, CDCl₃) δ 165.6 (C), 155.9 (C), 138.0 (C), 137.7 (C), 137.4 (C), 136.2 (C), 135.0 (C), 133.4 (CH), 133.2 (C), 133.0 (C), 129.8 (CH), 129.3 (C), 128.45 (CH), 128.42 (CH), 128.41 (CH), 128.38 (CH), 128.3 (CH), 128.1 (CH), 128.0 (CH), 127.9 (CH), 127.79 (CH), 127.76 (CH), 127.7 (CH), 127.6 (CH), 126.2 (CH), 126.1 (CH), 126.0 (CH), 100.4 (CH), 99.2 (CH), 80.4 (CH), 79.6 (CH), 78.9 (CH), 78.1 (CH), 77.0 (CH), 75.3 (CH₂), 75.2 (CH₂), 74.8 (CH₂), 74.1 (CH), 73.6 (CH₂), 72.1 (CH), 68.5 (CH₂), 67.0 (CH₂), 65.6 (CH₂), 54.7 (CH); HRMS (FAB, MNa⁺) calcd for C₅₉H₅₇NO₁₂Na 994.3778, found 994.3757. 75: ¹H NMR (400 MHz, CDCl₃) δ 8.00 (dd, J=8.4, 1.3 Hz, 2H, BzH), 7.95 (dd, J=8.4, 1.3 Hz, 2H, BzH), 7.67-7.53 (m, 5H, ArH), 7.49 (s, 1H, ArH), 7.44-7.35 (m, 6H, ArH), 7.32-7.09 (m, 12H, ArH), 7.04-6.98 (m, 2H, ArH), 6.90-6.88 (m, 2H, ArH), 5.60 (dd, J=10.5, 9.5 Hz, 1H, H-3′), 5.30 (d, J=1.6 Hz, 1H, H-1), 5.21 (d, J=3.7 Hz, 1H, H-1′), 5.11 (d, J=10.5 Hz, 1H, NH), 4.85, 4.55 (ABq, J=12.4 Hz, 2H, CH₂Ar), 4.83, 4.68 (ABq, J=10.9 Hz, 2H, CH₂Ar), 4.71 (s, 2H, CH₂Ar), 4.62 (s, 2H, CH₂Ar), 4.58 (dd, J=5.2, 4.0 Hz, 1H, H-5), 4.58-4.52 (m, 2H, H-6′a, H-6′b), 4.22 (td, J=10.5, 3.7 Hz, 1H, H-2′), 4.09 (d, J=7.8 Hz, 1H, H-6a), 4.01 (ddd, J=9.5, 3.7, 2.9 Hz, 1H, H-5′), 3.88 (t, J=9.5 Hz, 1H, H-4′), 3.86 (dd, J=8.2, 4.0 Hz, 1H, H-4), 3.77 (dd, J=8.2, 7.8 Hz, 1H, H-3), 3.73 (dd, J=7.8, 5.2 Hz, 1H, H-6b), 3.49 (dd, J=7.8, 1.6 Hz, 1H, H-2); ¹³C NMR (125 MHz, CDCl₃) δ 166.4 (C), 166.1 (C), 155.9 (C), 137.9 (C), 137.7 (C), 135.9 (C), 134.0 (C), 133.4 (CH), 133.2 (CH), 133.02 (C), 132.96 (C), 129.9 (CH), 129.6 (CH), 129.5 (C), 129.3 (C), 128.5 (CH), 128.44 (CH), 128.38 (CH), 128.2 (CH), 128.0 (CH), 127.81 (CH), 127.75 (CH), 127.7 (CH), 127.64 (CH), 127.58 (CH), 127.5 (CH), 127.4 (CH), 126.1 (CH), 126.0 (CH), 125.9 (CH), 99.8 (CH), 99.3 (CH), 83.1 (CH), 81.0 (CH), 79.0 (CH), 75.7 (CH), 75.5 (CH₂), 75.1 (CH₂), 74.1 (CH), 73.7 (CH), 72.8 (CH₂), 70.2 (CH), 66.7 (CH₂), 65.5 (CH₂), 63.1 (CH₂), 54.1 (CH); HRMS (FAB, MH⁺) calcd for C₅₉H₅₆NO₁₃ 986.3752, found 986.3762. 76: ¹H NMR (400 MHz, CDCl₃) δ 7.90 (d, J=7.2 Hz, 2H, BzH), 7.78-7.69 (m, 3H, ArH), 7.66 (s, 1H, ArH), 7.55-7.48 (m, 1H, ArH), 7.46-7.34 (m, 5H, ArH), 7.32-7.17 (m, 20H, ArH), 5.28 (d, J=1.3 Hz, 1H, H-1), 5.11, 4.78 (ABq, J=12.1 Hz, 2H, CH₂Ar), 5.06 (d, J=3.6 Hz, 1H, H-1′), 4.99, 4.79 (ABq, J=10.8 Hz, 2H, CH₂Ar), 4.84-4.74 (m, 3H, NH, CH₂Ar), 4.68-4.62 (m, 2H, CH₂Ar), 4.60 (s, 2H, CH₂Ar), 4.55 (t, J=4.2 Hz, 1H, H-5), 4.53 (dd, J=12.0, 1.8 Hz, 1H, H-6′a), 4.43 (dd, J=12.0, 4.7 Hz, 1H, H-6′b), 4.04 (td, J=10.1, 3.6 Hz, 1H, H-2′), 3.97 (d, J=7.7 Hz, 1H, H-6a), 3.83 (ddd, J=9.3, 4.7, 1.8 Hz, 1H, H-5′), 3.77 (dd, J=8.5, 4.2 Hz, 1H, H-4), 3.71 (dd, J=8.5, 7.7 Hz, 1H, H-3), 3.68 (t, J=9.3 Hz, 1H, H-4′), 3.66 (dd, J=7.7, 4.2 Hz, 1H, H-6b), 3.62 (dd, J=10.1, 9.3 Hz, 1H, H-3′), 3.48 (dd, J=7.7, 1.3 Hz, 1H, H-2); ¹³C NMR (125 MHz, CDCl₃) δ 166.1 (C), 155.9 (C), 138.0 (C), 137.8 (C), 137.6 (C), 136.2 (C), 134.6 (C), 133.2 (CH), 133.1 (CH), 129.5 (CH), 128.52 (CH), 128.45 (CH), 128.3 (CH), 128.13 (CH), 128.10 (CH), 128.0 (CH), 127.9 (CH), 127.8 (CH), 127.7 (CH), 127.6 (CH), 127.3 (CH), 126.2 (CH), 126.1 (CH), 126.0 (CH), 100.3 (CH), 99.3 (CH), 83.1 (CH), 80.5 (CH), 80.4 (CH), 79.3 (CH), 77.8 (CH), 75.4 (CH₂×2), 75.1 (CH₂), 74.0 (CH), 72.8 (CH₂), 70.5 (CH), 67.0 (CH₂), 65.4 (CH₂), 63.2 (CH₂), 54.8 (CH); HRMS (FAB, MH⁺) calcd for C₅₉H₅₈NO₁₂ 972.3959, found 972.3964. 77: ¹H NMR (400 MHz, CDCl₃) δ 7.94 (dd, J=8.2, 1.0 Hz, 2H, BzH), 7.69-7.58 (m, 2H, ArH), 7.56-7.50 (m, 2H, ArH), 7.43-7.25 (m, 15H, ArH), 7.23-7.13 (m, 5H, ArH), 7.12-7.07 (m, 2H, ArH), 7.04-6.97 (m, 2H, ArH), 6.92-6.86 (m, 2H, ArH), 5.51 (dd, J=10.5, 9.4 Hz, 1H, H-3′), 5.30 (d, J=1.4 Hz, 1H, H-1), 5.22 (d, J=3.8 Hz, 1H, H-1′), 5.07 (d, J=10.5 Hz, 1H, NH), 4.85, 4.55 (ABq, J=12.4 Hz, 2H, CH₂Ar), 4.82, 4.69 (ABq, J=10.9 Hz, 2H, CH₂Ar), 4.64 (s, 2H, CH₂Ar), 4.64, 4.59 (ABq, J=11.2 Hz, 2H, CH₂Ar), 4.62, 4.51 (ABq, J=12.3 Hz, 2H, CH₂Ar), 4.60 (dd, J=5.0, 4.2 Hz, 1H, H-5), 4.21 (td, J=10.5, 3.8 Hz, 1H, H-2′), 4.06 (d, J=7.8 Hz, 1H, H-6a), 3.90 (t, J=9.4 Hz, 1H, H-4′), 3.85 (dd, J=8.5, 4.2 Hz, 1H, H-4), 3.81 (ddd, J=9.4, 2.4, 1.5 Hz, 1H, H-5′), 3.78 (dd, J=8.5, 7.8 Hz, 1H, H-3), 3.76 (dd, J=12.7, 2.4 Hz, 1H, H-6′a), 3.71 (dd, J=7.8, 5.0 Hz, 1H, H-6b), 3.67 (dd, J=12.7, 1.5 Hz, 1H, H-6′b), 3.50 (dd, J=7.8, 1.4 Hz, 1H, H-2); ¹³C NMR (125 MHz, CDCl₃) δ 166.5 (C), 155.8 (C), 138.0 (C), 137.7 (C), 137.6 (C), 136.0 (C), 134.6 (C), 133.2 (CH), 133.0 (C), 132.9 (C), 129.8 (CH), 129.4 (CH), 128.5 (CH), 128.4 (CH), 128.2 (CH), 128.1 (CH), 128.0 (CH), 127.93 (CH), 127.87 (CH), 127.84 (CH), 127.76 (CH), 127.7 (CH), 127.64 (CH), 127.57 (CH), 127.5 (CH), 127.1 (CH), 125.99 (CH), 125.96 (CH), 125.9 (CH), 99.9 (CH), 99.3 (CH), 83.1 (CH), 81.0 (CH), 79.2 (CH), 75.8 (CH), 75.5 (CH₂), 75.0 (CH₂), 74.1 (CH), 73.8 (CH), 73.6 (CH₂), 72.8 (CH₂), 71.7 (CH), 68.3 (CH₂), 66.6 (CH₂), 65.6 (CH₂), 54.0 (CH); HRMS (FAB, MNa⁺) calcd for C₅₉H₅₇NO₁₂Na 994.3778, found 994.3782. 78: ¹H NMR (400 MHz, CDCl₃) δ 7.84-7.72 (m, 3H, ArH), 7.57 (s, 1H, ArH), 7.50-7.43 (m, 2H, ArH), 7.36-7.20 (m, 26H, ArH), 5.29 (d, J=1.2 Hz, 1H, H-1), 5.12 (d, J=12.2 Hz, 1H, CH₂Ar), 5.08 (d, J=3.7 Hz, 1H, H-1′), 4.92, 4.78 (ABq, J=10.9 Hz, 2H, CH₂Ar), 4.81-4.71 (m, 3H, NH, CH₂Ar), 4.70-4.58 (m, 6H, H-5, CH₂Ar), 4.54, 4.46 (ABq, J=12.2 Hz, 2H, CH₂Ar), 4.03 (td, J=10.1, 3.7 Hz, 1H, H-2′), 3.96 (d, J=7.8 Hz, 1H, H-6a), 3.77 (dd, J=8.1, 4.1 Hz, 1H, H-4), 3.72-3.60 (m, 5H, H-6b, H-3′, H-4′, H-6′a, H-6′b), 3.65 (dd, J=8.1, 7.6 Hz, 1H, H-3), 3.59-3.52 (m, 1H, H-5′), 3.49 (dd, J=7.6, 1.2 Hz, 1H, H-2); ¹³C NMR (125 MHz, CDCl₃) δ 155.9 (C), 138.0 (C), 137.9 (C), 137.7 (C), 137.6 (C), 136.2 (C), 135.0 (C), 133.2 (C), 133.0 (C), 128.5 (CH), 128.41 (CH), 128.37 (CH), 128.2 (CH), 128.1 (CH), 127.98 (CH), 127.95 (CH), 127.9 (CH), 127.8 (CH), 127.73 (CH), 127.69 (CH), 127.6 (CH), 127.0 (CH), 126.1 (CH), 126.04 (CH), 126.01 (CH), 100.4 (CH), 99.3 (CH), 83.0 (CH), 80.5 (CH), 80.2 (CH), 79.6 (CH), 78.1 (CH), 75.3 (CH₂), 75.19 (CH₂), 75.17 (CH₂), 74.0 (CH), 73.5 (CH₂), 72.8 (CH₂), 71.9 (CH), 68.5 (CH₂), 66.9 (CH₂), 65.5 (CH₂), 54.7 (CH); HRMS (FAB, MH⁺) calcd for C₅₉H₆₀NO₁₁ 958.4166, found 958.4171.

### Preparation of Sulfonated Disaccharide Compounds

Transformations of two representative synthons into their corresponding sulfated disaccharides are exemplified in Scheme 6.

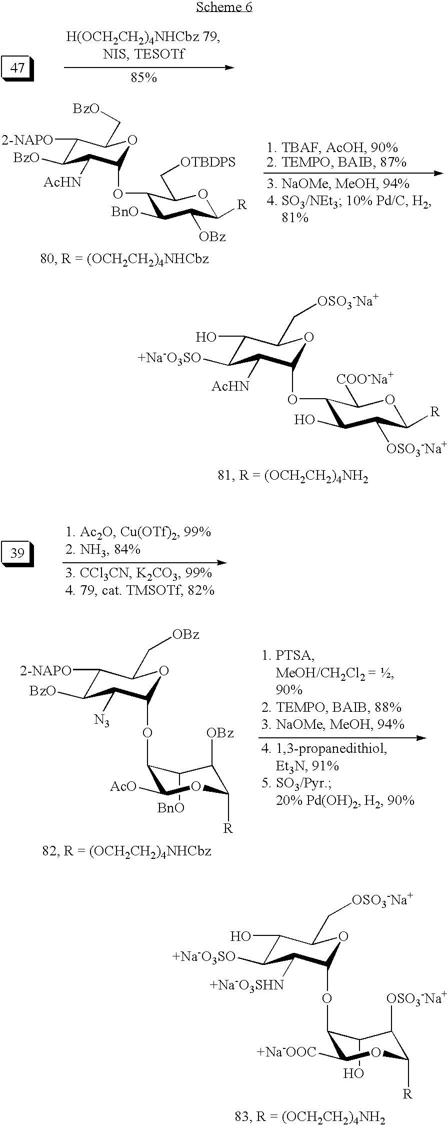

Synthesis of Compound 80: A mixture of the disaccharide donor 47 (92 mg, 0.07 mmol), the linker 79 (28 mg, 0.09 mmol), N-iodosuccinimide (20 mg, 0.09 mmol) and freshly dried 3 Å molecular sieves (150 mg) in anhydrous dichloromethane (1.5 mL) was kept stirring at room temperature for 30 min under nitrogen. The reaction flask was cooled down to −78° C., and triethylsilyl trifluoromethanesulfonate (3 μL) was slowly added to the solution. The mixture was gradually warm up to −10° C. over 1.5 h, and triethylamine was added to quench the reaction. The resulting mixture was filtered through celite, the flask and celite pad were washed with EtOAc, and the filtrate was sequentially washed with 5% Na₂S₂O₃ (aq), saturated NaHCO₃ (aq) and brine. The organic layer was dried over MgSO₄, filtered, and concentrated in vacuo to get the crude product, which was purified by flash column chromatography (EtOAc/Hex=1/1) on silica gel to get the linker-attached disaccharide 80 (93 mg, 86%). ¹H NMR (400 MHz, CDCl₃) δ 8.10-7.99 (m, 4H, ArH), 7.93 (dd, J=8.4, 1.3 Hz, 2H, ArH), 7.74-7.67 (m, 2H, ArH), 7.67-7.50 (m, 8H, ArH), 7.49 (s, 1H, ArH), 7.46-7.22 (m, 18H, ArH), 7.26-7.22 (m, 1H, ArH), 7.21-7.15 (m, 3H, ArH), 7.06-6.95 (m, 3H, ArH), 6.69 (d, J=10.0 Hz, 1H, NHAc), 5.76 (dd, J=10.9, 9.2 Hz, 1H, H-3′), 5.40 (dd, J=9.2, 8.2 Hz, 1H, H-2), 5.33 (d, J=3.2 Hz, 1H, H-1′), 5.30 (s, 1H, NHCbz), 5.07 (s, 2H, CH₂Ar), 4.82 (s, 2H, CH₂Ar), 4.68 (s, 2H, CH₂Ar), 4.60 (d, J=8.2 Hz, 1H, H-1), 4.60-4.55 (m, 1H, H-2′), 4.52 (dd, J=12.0, 3.8 Hz, 1H, H-6′a), 4.42 (dd, J=12.0, 1.9 Hz, 1H, H-6′b), 4.30-4.23 (m, 2H, H-6a, H-5′), 4.08 (t, J=9.2 Hz, 1H, H-4), 3.96 (t, J=9.2 Hz, 2H, H-3, H-4′), 3.92-3.87 (m, 1H, ethylene glycol moiety), 3.85 (d, J=11.0 Hz, 1H, H-6b), 3.67-3.37 (m, 1H, ethylene glycol moiety), 3.37-3.26 (m, 5H, H-5, ethylene glycol moiety), 0.97 (s, 9H, t-Bu), 0.94 (s, 3H, NHAc); ¹³C NMR (125 MHz, CDCl₃) δ 170.7 (C), 166.5 (C), 166.1 (C), 165.3 (C), 156.4 (C), 136.6 (C), 136.3 (C), 136.0 (CH), 135.4 (CH), 134.2 (C), 133.34 (CH), 133.25 (CH), 133.1 (CH), 133.00 (C), 132.96 (C), 132.7 (C), 129.8 (CH), 129.7 (CH), 129.63 (CH), 129.59 (CH), 129.0 (CH), 128.52 (CH), 128.49 (CH), 128.46 (CH), 128.4 (CH), 128.3 (CH), 128.2 (CH), 128.0 (CH), 127.8 (CH), 127.6 (CH), 127.54 (CH), 127.51 (CH), 127.4 (CH), 126.1 (CH), 125.94 (CH), 125.91 (CH), 100.8 (CH), 100.1 (CH), 81.8 (CH), 78.0 (CH), 76.0 (CH), 75.8 (CH₂), 75.6 (CH), 74.9 (CH₂), 74.5 (CH), 73.8 (CH), 70.9 (CH), 70.6 (CH₂), 70.4 (CH₂), 70.3 (CH₂), 70.2 (CH₂), 70.1 (CH₂), 70.0 (CH₂), 68.7 (CH₂), 66.6 (CH₂), 63.3 (CH₂), 62.2 (CH₂), 52.4 (CH), 40.8 (CH₂), 26.7 (t-Bu), 21.5 (CH₃), 19.2 (C); HRMS (FAB, MNa⁺) calcd for C₈₅H₉₂N₂O₁₉SiNa 1495.5961, found 1459.5963.

Synthesis of Compound 81: (1) Desilylation. To a solution of compound 80 (84 mg, 0.06 mmol), and acetic acid (7 μL, 0.11 mmol) in tetrahydrofuran (1 mL) was added a 1 M solution of tetrabutylammonium fluoride in tetrahydrofuran (114 μL, 0.11 mmol). The reaction was kept stirring at room temperature for 8 h, and the mixture was evaporated under reduced pressure. The residue was directly purified by flash column chromatography (EtOAc/Hex=2/1) on silica gel to afford the desired 6-alcohol (63 mg, 90%). (2) TEMPO Oxidation. To a solution of this 6-alcohol (43 mg, 0.04 mmol) in a mixed solvent of H₂O and CH₂Cl₂ (½ ratio, 750 μL) was consecutively added 2,2,6,6-tetramethyl-1-piperidinyloxy (1 mg, 0.01 mmol) and (bisacetoxy)iodobenzene (28 mg, 0.09 mmol) at room temperature. After stirring for 2 h, the solution was diluted with EtOAc, and the mixture was sequentially washed with 5% Na₂S₂O₃ (aq), saturated NaHCO₃ (aq) and brine. The organic layer was dried over MgSO₄, filtered, and concentrated in vacuo to get a residue. Purification this residue via flash column chromatography (MeOH/CHCl₃=1/7) on silica gel furnished the corresponding carboxylic acid (38 mg, 87%). (3) Debenzoylation. Sodium methoxide (3 mg, 0.06 mmol) was added to a solution of this carboxylic acid (32 mg, 0.03 mmol) in MeOH (1 mL) at room temperature. The mixture was stirred overnight, and the reaction solution was neutralized by acidic DOWEX 50WX4 resin. The mixture was filtered through filter paper, and the filtrate was concentrated in vacuo to get the crude product, which was purified by flash column chromatography (MeOH/CHCl₃=1/5) on silica gel to provide the triol compound (23 mg, 94%). (4) O-Sulfonation. A solution of this triol compound (61 mg, 0.07 mmol) and sulfur trioxide-triethylamine complex (249 mg, 1.38 mmol) in anhydrous N,N-dimethylformamide (700 μL) was stirred at 60° C. for 3 d. The reaction flask was cooled down to room temperature, the solution was quenched with saturated NaHCO₃ (aq), and the mixture was concentrated in vacuo. The white solid residue was treated with MeOH and CH₂Cl₂ (½ ratio, 10 mL), the mixture was filtered through paper, and the filtrate was evaporated under reduced pressure. This three-stepped protocol was repeated for 3 times to get the crude O-sulfonated product, which was directly used for the ensuing reaction without further purification. (5) Hydrogenolysis. A mixture of this crude O-sulfonated compound, 10% Pd/C (166 mg) and acetic acid (7.5 μL, 0.13 mmol) in a mixed solvent of MeOH and H₂O (7/3 ratio, 3 mL) was equipped with a hydrogen balloon, and the reaction solution was stirred for 1 d. The mixture was filtered through celite, and the mixture was concentrated in vacuo. The residue was dissolved in MeOH and H₂O (1/1 ratio), the mixture was eluted on a Sephadex G-25 (Na⁺) column, and the collected portion was lyophilized to provide 81 (48 mg, 81% in two steps). ¹H NMR (500 MHz, CDCl₃) δ 5.44 (d, J=3.7 Hz, 1H, H-1′), 4.70 (d, J=7.8 Hz, 1H, H-1), 4.47 (dd, J=10.6, 9.2 Hz, 1H, H-3′), 4.40 (dd, J=11.0, 2.4 Hz, 1H, H-6′a), 4.20 (dd, J=11.0, 2.0 Hz, 1H, H-6′b), 4.16-4.09 (m, 2H, H-2, H-2′), 4.06-3.95 (m, 3H, H-3, H-5′, ethylene glycol moiety), 3.90-3.82 (m, 3H, H-4, H-5, ethylene glycol moiety), 3.81-3.69 (m, 13H, H-4′, ethylene glycol moiety), 3.24 (t, J=5.0 Hz, 2H, ethylene glycol moiety); ¹³C NMR (125 MHz, CDCl₃) δ 174.7 (C), 174.3 (C), 100.6 (CH), 97.6 (CH), 80.4 (CH), 79.4 (CH), 76.50 (CH), 76.45 (CH), 75.0 (CH), 70.0 (CH), 69.7 (CH₂), 69.5 (CH₂), 69.3 (CH₂), 69.2 (CH₂), 67.6 (CH), 66.3 (CH₂), 66.1 (CH₂), 52.0 (CH), 39.1 (CH₂), 22.0 (CH₃); HRMS (FAB, MH⁺) calcd for C₂₂H₃₇N₂O₂₄S₃Na₄ 901.0489, found 901.0477.

Synthesis of Compound 82: (1) Acetolysis. A mixture of 39 (1.50 g, 1.68 mmol), fleshly dried copper (II) trifluoromethanesulfonate (61 mg, 0.17 mmol), and acetic anhydride (15 mL) was stirred at 0° C. for 18 h under nitrogen. Methanol (10 mL) was added to quench the excess acetic anhydride, and the whole solution was kept stirring at room temperature for another 0.5 h. The resulting mixture was concentrated in vacuo, and the residue was dissolved in ethyl acetate followed by sequential wash with saturated NaHCO₃ (aq) and brine. The organic phase was dried over magnesium sulfate, filtered, and concentrated in vacuo. The residue was purified by flash column chromatography (EtOAc/Hex=2/3) on silica gel to yield the 1,6-diacetate compound (1.65 g, α/β=1.6/1, 99%). (2) Deacetylation. Ammonia gas was passed through a solution of this 1,6-diacetate (1.46 g, 1.47 mmol) in a mixed solvent of THF and MeOH (10/1 ratio, 77 mL) for 2.5 min at 0° C. The gas valve was closed, and the mixture was kept stirring at the same temperature for another 8 h. After warming up to room temperature, the solvent was removed under reduced pressure, and the residue was purified by flash column chromatography (EtOAc/Hex=1/2) on silica gel to give the corresponding 1-alcohol (1.17 g, α/β=1/1, 84%). (3) Trichloroacetimidaton. To a mixture of this 1-alcohol (859 mg, 0.90 mmol) in dichloromethane (10 mL) was consecutively added potassium carbonate (622 mg, 4.50 mmol) and trichloroacetonitrile (902 μL, 9.00 mmol) at room temperature under nitrogen. After stirring for 16 h, the reaction solution was filtered through celite, the filtrate was diluted by water, and the mixture was extracted with ethyl acetate for three times. The combined organic layers were washed with brine, dried over magnesium sulfate, filtered, and concentrated in vacuo to furnish the crude trichloroacetimidate (986 mg, α/β=1/1.3, 99%), which was directly used for the ensuing reaction without further purification. (4) Glycosylation. A mixture of this trichloroacetimidate (978 mg, 0.89 mmol), linker 79 (435 mg, 1.33 mmol), and freshly dried 4 Å molecular sieves (1.50 g) in dichloromethane (20 mL) was stirred at room temperature for 1 h under nitrogen. The reaction flask was cooled down to −78° C., trimethylsilyl trifluoromethanesulfonate (32 μL, 0.18 mmol) was added to the solution, and the mixture was gradually warmed up to room temperature. After stirring for 1.5 h, triethylamine (25 μL, 0.18 mmol) was added to quench the reaction, and the mixture was filtered through celite. The filtrate was concentrated in vacuo to give a residue, which was purified by flash column chromatography (EtOAc/Hex=1/1) to provide the linker attached disaccharide 82 (913 mg, 82%). ¹H NMR (400 MHz, CDCl₃) δ 8.06-8.01 (m, 2H, ArH), 7.96-7.90 (m, 4H, ArH), 7.61-7.45 (m, 5H, ArH), 7.44 (s, 1H, ArH), 7.37-7.11 (m, 20H, ArH), 5.74 (dd, J=10.6, 9.1 Hz, 1H, H-3′), 5.43 (t, J=5.0 Hz, 1H, NHCbz), 5.16 (dd, J=4.3, 2.7 Hz, 1H, H-2), 5.03 (s, 2H, CH₂Ar), 4.97 (d, J=2.7 Hz, 1H, H-1), 4.94 (d, J=3.7 Hz, 1H, H-1′), 4.78, 4.64 (ABq, J=11.4 Hz, 2H, CH₂Ar), 4.67, 4.63 (ABq, J=11.1 Hz, 2H, CH₂Ar), 4.60-4.46 (m, 3H, H-6a, H-6′a, H-6′b), 4.44-4.38 (m, 1H, H-5), 4.32 (dd, J=11.4, 4.6 Hz, 1H, H-6b), 4.18 (ddd, J=9.9, 4.1, 2.1 Hz, 1H, H-5′), 4.01 (t, J=4.3 Hz, 1H, H-3), 3.91 (t, J=4.3 Hz, 1H, H-4), 3.84-3.78 (m, 1H, ethylene glycol moiety), 3.76 (dd, J=9.9, 9.1 Hz, 1H, H-4′), 3.63-3.40 (m, 13H, ethylene glycol moiety), 3.34 (dd, J=10.6, 3.7 Hz, 1H, H-2′), 3.31 (q, J=5.0 Hz, 2H, ethylene glycol moiety), 2.03 (s, 3H, Ac); ¹³C NMR (100 MHz, CDCl₃) δ 170.4 (C), 165.9 (C), 165.6 (C), 164.9 (C), 156.3 (C), 137.4 (C), 136.5 (C), 134.0 (C), 133.3 (CH), 133.0 (CH), 132.9 (CH), 132.83 (C), 132.78 (C), 129.61 (CH), 129.57 (CH), 129.5 (C), 129.4 (CH), 129.3 (C), 129.1 (C), 128.29 (CH), 128.28 (CH), 128.2 (CH), 128.1 (CH), 127.9 (CH), 127.8 (CH), 127.63 (CH), 127.62 (CH), 127.5 (CH), 127.1 (CH), 98.1 (CH), 97.3 (CH), 75.7 (CH), 74.7 (CH₂), 73.9 (CH), 73.3 (CH), 72.8 (CH), 72.7 (CH₂), 70.5 (CH₂), 70.3 (CH₂), 70.2 (CH₂), 70.02 (CH₂), 69.98 (CH×2), 69.9 (CH₂), 69.8 (CH₂), 67.5 (CH₂), 66.6 (CH), 66.4 (CH₂), 62.8 (CH₂), 62.5 (CH₂), 61.5 (CH), 40.7 (CH₂), 20.7 (CH₃); HRMS (FAB, MNa⁺) calcd for C₆₉H₇₂N₄O₁₉Na 1283.4688, found 1283.4679.

Synthesis of Compound 83: (1) Deacetylation p-Toluenesulfonic acid monohydrate (325 mg, 1.71 mmol) was added to a solution of 82 (719 mg, 0.57 mmol) in a mixed solvent of CH₂Cl₂ and MeOH (2/1 ratio, 9 mL) at room temperature under nitrogen. After stirring for 3 d, triethylamine (238 μL, 1.71 mmol) was added to quench the reaction, and the whole mixture was removed under reduced pressure. The residue was purified by flash column chromatography (EtOAc/Hex=2/1) on silica gel, and the corresponding 6-alcohol (682 mg) was obtained in 98% yield. (2) TEMPO Oxidation. To a solution of this 6-alcohol (44 mg, 0.04 mmol) in a mixed solvent of H₂O and CH₂Cl₂ (½ ratio, 750 μL) was added 2,2,6,6-tetramethyl-1-piperidinyloxy (1 mg, 0.01 mmol) and (bisacetoxy)iodobenzene (29 mg, 0.09 mmol) at room temperature. The reaction was continuously stirred for 2 h, the mixture was diluted with EtOAc, and the resulting solution was sequentially washed with 5% Na₂S₂O₃ (aq), saturated NaHCO₃ (aq) and brine. The organic layer was dried over MgSO₄, filtered, and concentrated in vacuo to get the crude product, which was purified by flash column chromatography (MeOH/CHCl₃=1/9) on silica gel to furnish the desired carboxylic acid compound (39 mg, 88%). (3) Debenzoylation. Sodium methoxide (5 mg, 0.09 mmol) was added to a solution of this carboxylic acid (36 mg, 0.03 mmol) in MeOH at room temperature. After stirring overnight, acidic DOWEX 50WX4 resin was added to neutralize the solution, the resulting mixture was filtered through paper, and the filtrate was concentrated in vacuo. Purification of this residue by flash column chromatography (MeOH/CHCl₃=1/7) on silica gel afforded the triol compound (25 mg, 94%). (4) Azido Reduction. This triol (23 mg, 0.03 mmol) was dissolved in methanol (500 μL), and triethylamine (70 μL, 0.50 mmol) and 1,3-propanedithiol (51 μL, 0.50 mmol) were consecutively added to the solution. The reaction flask was protected by aluminum foil to avoid the light, and the mixture was kept stirring at room temperature for 2 days. The whole solution was filtered through celite, and the filtrate was concentrated in vacuo. The residue was purified by flash column chromatography (MeOH/CHCl₃=1/5) on silica gel to yield the corresponding amine (20 mg, 91%). (5) N- and O-Sulfonation. Sulfur trioxide/pyridine complex (86 mg, 0.54 mmol) was added to a solution of this amine (24 mg, 0.03 mmol) in pyridine (1.5 mL) under nitrogen, the reaction flask was protected from light, and the mixture was stirred at room temperature for 1 d and then at 55° C. for another 1 day. The reaction solution was cooled down to room temperature, saturated NaHCO₃ (aq) was added to quench the reaction, and the resulting solution was concentrated in vacuo. The white solid residue was treated with MeOH and CH₂Cl₂ (½ ratio, 10 mL), the mixture was filtered, and the filtrate was concentrated in vacuo. This three-stepped procedure was repeated for 3 times, and the residue was purified by flash column chromatography (MeOH/CHCl₃=1/3) to get the N- and O-sulfonated compound (30 mg, 91%). (6) Hydrogenolysis. A solution of this N- and O-sulfonated compound (30 mg, 0.02 mmol) and 20% Pd(OH)₂ (166 mg) in a phosphate buffer (20 mM, pH=7.0, 3 mL) was equipped with a hydrogen balloon, and the mixture was stirred at room temperature for 1 d. The whole mixture was filtered through celite, and the filtrate was concentrated in vacuo. The residue was dissolved in a mixed MeOH and H₂O (1/1 ratio), the mixture was eluted on a Sephadex G-25 column, and the collected portion was lyophilized to afford 83 (23 mg, 99%). ¹H NMR (400 MHz, CDCl₃) δ 5.45 (d, J=3.7 Hz, 1H, H-1′), 5.25 (s, 1H, H-1), 4.69 (s, 1H, H-5), 4.59 (dd, J=10.3, 9.4 Hz, 1H, H-3′), 4.45-4.37 (m, 2H, H-2, H-6′a), 4.33 (s, 1H, H-3), 4.28 (dd, J=11.0, 1.5 Hz, 1H, H-6′b), 4.21 (s, 1H, H-4), 4.12-4.06 (m, 1H, H-5′), 3.94 (ddd, J=11.5, 5.5, 3.0 Hz, 1H, ethylene glycol moiety), 3.88-3.72 (m, 14H, H-4′, ethylene glycol moiety), 3.58 (dd, J=10.3, 3.7 Hz, 1H, H-2′), 3.27 (t, J=5.0 Hz, 2H, ethylene glycol moiety); ¹³C NMR (100 MHz, CDCl₃) δ 175.0 (C), 98.5 (CH), 92.5 (CH), 78.5 (CH), 72.7 (CH), 71.0 (CH), 69.9 (CH), 69.7 (CH₂), 69.64 (CH₂), 69.56 (CH₂), 67.5 (CH₂), 67.2 (CH), 66.46 (CH), 66.42 (CH₂), 66.2 (CH₂), 55.6 (CH), 53.4 (CH), 39.2 (CH₂).

### OTHER EMBODIMENTS

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. A disaccharide compound of general formula: 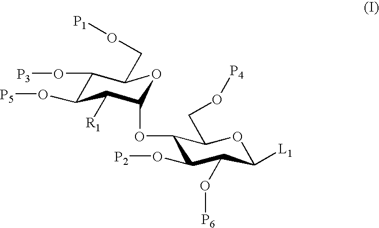
wherein
each of P₁, P₂, P₅, and P₆, independently, is arylalkyl, arylcarbonyl, alkylcarbonyl, heteroarylakyl, or heteroarylcarbonyl;
P₃ is arylalkyl or heteroarylalkyl;
P₄ is silyl;
L₁ is alkoxy, aryloxy, cycloalkyloxy, heteroaryloxy, heterocycloalkyloxy, alkylthio, arylthio, cycloalkylthio, heteroarylthio, heterocycloalkylthio, carbonyloxy, or halo; and
R₁ is azido or amino optionally substituted by an amino protecting group.

2. The disaccharide compound of claim 1, wherein each of P₁, P₂, P₅, P₆, independently, is benzoyl or benzyl.

3. The disaccharide compound of claim 2, wherein P₂ is benzyl, P₃ is 2-naphthylmethyl, and P₄ is tert-butyldiphenylsilyl.

4. The disaccharide compound of claim 3, wherein L₁ is p-methylphenylthio.

5. The disaccharide compound of claim 4, wherein R₁ is N₃, acetamido, or benzyloxycarbonylamino.

6. The disaccharide compound of claim 1, wherein P₂ is benzyl, P₃ is 2-naphthylmethyl, and P₄ is tert-butyldiphenylsilyl.

7. The disaccharide compound of claim 1, wherein L₁ is p-methylphenylthio.

8. The disaccharide compound of claim 1, wherein R₁ is N₃, acetamido, or benzyloxycarbonylamino.

9. A process for preparing the disaccharide compound of claim 1, comprising:
reacting a first monosaccharide having the general formula of 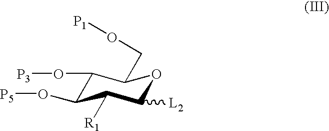
with a second monosaccharide having the general formula of 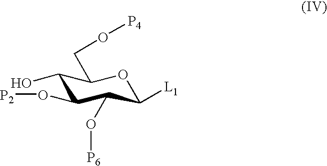
wherein
each of P₁, P₂, P₅, and P₆ independently, is arylalkyl, arylcarbonyl, alkylcarbonyl, heteroarylakyl, or heteroarylcarbonyl;
P₃ is arylalkyl or heteroarylalkyl;
P₄ is silyl;
L₁ is alkoxy, aryloxy, cycloalkyloxy, heteroaryloxy, heterocycloalkyloxy, alkylthio, arylthio, cycloalkylthio, heteroarylthio, heterocycloalkylthio, carbonyloxy, or halo;
L₂ is a leaving group; and
R₁ is azido or amino optionally substituted by an amino protecting group.

10. The process of claim 9, wherein each of P₁, P₅, P₆, independently, is benzoyl or benzyl; P₂ is benzyl; P₃ is -naphthylmethyl; P₄ is tert-butyldiphenylsilyl; L₁ is p-methylphenylthio; and R₁ is N₃, acetamido, or benzyloxycarbonylamino.

11. The process of claim 10, wherein L₂ is 2,2,2-trichloro-1-iminoethoxy.

12. The process of claim 11, wherein the reaction is carried out in the presence of silver trifluoromethanesulfonate or trimethylsilyl trifluoromethanesulfonate.

13. A disaccharide compound of general formula: 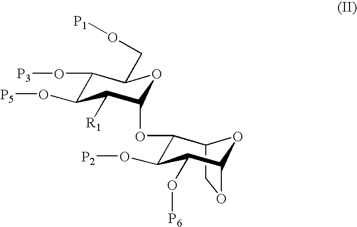
wherein
each of P₁, P₂, P₅, and P₆, independently, is arylalkyl, arylcarbonyl, alkylcarbonyl, heteroarylakyl, or heteroarylcarbonyl;
P₃ is arylalkyl or heteroarylalkyl; and
R₁ is azido or amino optionally substituted by an amino protecting group.

14. The disaccharide compound of claim 13, wherein each of P₁, P₂, P₅, P₆, independently, is benzoyl or benzyl.

15. The disaccharide compound of claim 14, wherein P₂ is benzyl and P₃ is 2-naphthylmethyl.

16. The disaccharide compound of claim 15, wherein R₁ is N₃, acetamido, or benzyloxycarbonylamino.

17. The disaccharide compound of claim 13, wherein P₂ is benzyl and P₃ is 2-naphthylmethyl.

18. The disaccharide compound of claim 13, wherein R₁ is N₃, acetamido, or benzyloxycarbonylamino.

19. A process for preparing the disaccharide compound of claim 13, comprising:
reacting a first monosaccharide having the general formula of 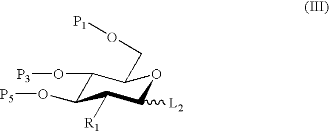
with a second monosaccharide having the general formula of 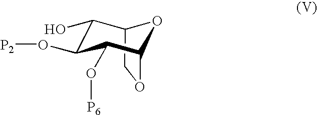
wherein
each of P₁ and P₂, P₅, and P₆, independently, is arylalkyl, arylcarbonyl, alkylcarbonyl, heteroarylakyl, or heteroarylcarbonyl;
P₃ is arylalkyl or heteroarylalkyl;
L₂ is a leaving group; and
R₁ is azido or amino optionally substituted by an amino protecting group.

20. The process of claim 19, wherein each of P₁, P₅, P₆, independently, is benzoyl or benzyl; P₂ is benzyl; P₃ is 2-naphthylmethyl; and R₁ is N₃, acetamido, or benzyloxycarbonylamino.

21. The process of claim 20, wherein L₂ is 2,2,2-trichloro-1-iminoethoxy.

22. The process of claim 21, wherein the reaction is carried out in the presence of silver trifluoromethanesulfonate or trimethylsilyl trifluoromethanesulfonate.
